(19) United States
(12) Patent Application Publication
(10) Pub. No.: US 2024/0076745 A1
(43) Pub. Date: Mar. 7, 2024

(54) MACHINE LEARNING CLASSIFICATION OF LUNG NODULES BASED ON GENE EXPRESSION

(71) Applicant: AMPEL BIOSOLUTIONS, LLC, Charlottesville, VA (US)

(72) Inventors: Prathyusha BACHALI, Redmond, WA (US); Amrie C. GRAMMER, Charlottesville, VA (US); Peter E. LIPSKY, Charlottesville, VA (US)

(21) Appl. No.: 18/269,920

(22) PCT Filed: Dec. 28, 2021
(86) PCT No.: PCT/US21/65348

§ 371 Date: Jun. 27, 2023

Related U.S. Application Data
(60) Provisional application No. 63/132,130, filed on Dec. 30, 2020.

(51) Int. Cl. C12Q 1/6886; G16H 50/20

(52) U.S. Cl. CPC C12Q 1/6886; G16H 50/20; C12Q 2600/158; G01N 2800/60

(57) ABSTRACT

The present disclosure provides systems and methods for machine learning classification of lung nodules based on gene expression data and clinical characteristics data. The method can include, a) obtaining a data set containing gene expression measurements of a biological sample from a patient of at least two lung disease-associated genes, and clinical characteristics data of one or more clinical characteristics of the patient; b) providing the data set as an input to a machine-learning model trained to generate an inference of whether the data set is indicative of a malignant lung nodule or a benign lung nodule; c) receiving, as an output of the machine-learning model, the inference indicating whether the data set is indicative of the malignant lung nodule or the benign lung nodule; and d) electronically outputting a report classifying the lung nodule of the patient as the malignant lung nodule or the benign lung nodule.

### CROSS-REFERENCE

This application claims priority to U.S. Provisional Patent Application No. 63/132,130, filed Dec. 30, 2020, incorporated in full herein by reference.

### BACKGROUND

Lung nodules are common, often detected in screenings of patients experiencing no symptoms of lung disease. Among subjects having lung nodules, only a fraction are eventually diagnosed with a cancer. Noncancerous causes of lung nodules can include e.g., mycobacterial or fungal infection, autoimmune diseases, air pollutants, and scarring from previous insult. Large lung nodules typically warrant an invasive biopsy or removal by thoracic surgery. The percentage of lung nodules eventually identified as cancerous has been estimated to be as low as 40%. Given the potential harm of biopsy or thoracic surgery, less invasive testing for lung cancer is needed. A simple noninvasive test, e.g., a blood test, would greatly reduce the potential for patient harm, and lower medical costs.

### SUMMARY

In an aspect, the present disclosure provides a method for assessing a lung nodule of a subject, comprising: (a) assaying a biological sample obtained or derived from the subject to produce a data set comprising gene expression measurements of the biological sample from each of a plurality of lung disease-associated genomic loci, wherein the plurality of lung disease-associated genomic loci comprises at least one gene selected from the group of genes listed in any one or more of Tables 1, 2, 3, 4, 5, 7, and 8; (b) analyzing the data set to classify the lung nodule of the subject as a malignant lung nodule or a benign lung nodule; and (c) electronically outputting a report indicative of the classification of the lung nodule of the subject as the malignant lung nodule or the benign lung nodule. Gene expression of the biological sample can be measured by, e.g., assaying RNA produced from genomic loci, e.g., lung disease-associated genes. The gene expression measurement in the biological sample can be performed using any suitable technique, such as any suitable RNA quantification technique, including but not limited to RNA-seq, Ampli-seq, and the like. In some embodiments, the dataset further comprises, clinical characteristics data of one or more clinical characteristics of the subject. In some embodiments, the one or more clinical characteristics are selected from the group of clinical characteristics listed in Table 6.

In some embodiments, the plurality of disease-associated genomic loci comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, or 180 genes selected from the group of genes listed in Table 1.

In some embodiments, the plurality of disease-associated genomic loci comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, or 175 genes selected from the group of genes listed in Table 2.

In some embodiments, the plurality of disease-associated genomic loci comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, or 60 genes selected from the group of genes listed in Table 3.

In some embodiments, the plurality of disease-associated genomic loci comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295 genes selected from the group of genes listed in Table 4.

In some embodiments, the plurality of disease-associated genomic loci comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, or 142 genes selected from the group of genes listed in Table 5.

In some embodiments, the plurality of disease-associated genomic loci comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7. In some embodiments, the genes are selected from BCAT1, CRCP, COA4, OVCA2, POM121, HLA-DPA1, VPS37C, MGST2, RNF220, HDAC3, NFE2L1, WDR20, CNPY4, HOXB2, C6orf120, TMEM8A, ASAP1-IT2, C15orf54, CD101, FNBP1, TECR, PROK2, SLC35B3, TDRD9, CLHC1, LPL, IFITM3, OGFOD3, EIF2B3, TMEM65, and MKRN3. In some embodiments, the plurality of disease-associated genomic loci comprises the genes BCAT1, CRCP, COA4, OVCA2, POM121, HLA-DPA1, VPS37C, MGST2, RNF220, HDAC3, NFE2L1, WDR20, CNPY4, HOXB2, C6orf120, TMEM8A, ASAP1-IT2, C15orf54, CD101, FNBP1, TECR, PROK2, SLC35B3, TDRD9, CLHC1, LPL, IFITM3, OGFOD3, EIF2B3, TMEM65, and MKRN3. In some embodiments, the plurality of disease-associated genomic loci consists of the genes BCAT1, CRCP, COA4, OVCA2, POM121, HLA-DPA1, VPS37C, MGST2, RNF220, HDAC3, NFE2L1, WDR20, CNPY4, HOXB2, C6orf120, TMEM8A, ASAP1-IT2, C15orf54, CD101, FNBP1, TECR, PROK2, SLC35B3, TDRD9, CLHC1, LPL, IFITM3, OGFOD3, EIF2B3, TMEM65, and MKRN3. These genes and those described herein are known to those of skill in the art, and described in the literature. Table A provides examples of Gene ID numbers for genes listed herein in the Tables, including Tables 7 and 8, as described in OMIM®—Online Mendelian Inheritance in Man (McKusick-Nathans Institute of Genetic Medicine, Johns Hopkins University School of Medicine, Baltimore, MD) and in the National Center for Biotechnology Information gene database (NCBI, U.S. National Library of Medicine 8600 Rockville Pike, Bethesda MD, 20894 USA), each incorporated by reference herein in their entirety.

TABLE A
Selected Genes Example Gene ID Numbers

| | OMIM | Entrez Gene ID |
|---|---|---|
| Predictor | No. | (NCBI) |
| BCAT1 | 113520 | 586 |
| CRCP | 606121 | 27297 |
| COA4 | 608016 | 51287 |
| OVCA2 | 607896 | 124641 |
| POM121 | 615753 | 9883 |
| HLA-DPA1 | 142880 | 3113 |
| VPS37C | 610038 | 55048 |
| MGST2 | 601733 | 4258 |
| RNF220 | 616136 | 55182 |
| HDAC3 | 605166 | 8841 |
| NFE2L1 | 163260 | 4779 |
| WDR20 | 617741 | 91833 |
| CNPY4 | 610047 | 245812 |
| HOXB2 | 142967 | 3212 |
| C6orf120 | 616987 | 387263 |
| TMEM8A | 619342 | 58986 |
| ASAP1-IT2 | — | 100507117 |
| C15orf54 (LINC02915) | — | 400360 |
| CD101 | 604516 | 9398 |
| FNBP1 | 606191 | 23048 |
| TECR | 610057 | 9524 |
| PROK2 | 607002 | 60675 |
| SLC35B3 | 610845 | 51000 |
| TDRD9 | 617963 | 122402 |
| CLHC1 | — | 130162 |
| LPL | 609708 | 4023 |
| IFITM3 | 605579 | 10410 |
| OGFOD3 (C17orf101) | — | 79701 |
| EIF2B3 | 606273 | 8891 |
| TMEM65 | 616609 | 157378 |
| MKRN3 | 603856 | 7681 |
| USP32P2 | — | 220594 |
| CD177 | 162860 | 57126 |
| QPCT | 607065 | 25797 |
| SCAF4 | 616023 | 57466 |
| SNRPD3 | 601062 | 6634 |
| BCL9L | 609004 | 283149 |
| THBS1 | 188060 | 7057 |
| SLC22A18AS | 603240 | 5003 |
| ARCN1 | 600820 | 372 |
| DHX16 | 603405 | 8449 |
| SATB1 | — | 6304 |
| ST6GAL1 | 109675 | 6480 |
| TDRD9 | 617963 | 122402 |
| ZNF831 | — | 128611 |
| MTCH1 | 610449 | 23787 |
| FAM86HP | — | 729375 |
| DHX8 | 600396 | 1659 |
| RNF114 | 61245 | 55905 |
| DCTN4 | 614758 | 51164 |

In some embodiments, the plurality of disease-associated genomic loci comprises at least 2, 3, 4, 5, 6, 7, 8, 9, 10, 11, 12, 13, 14, 15, 16, 17, 18, 19, 20, or 21, genes selected from the group of genes listed in Table 8. In some embodiments, the genes are selected from BCAT1, USP32P2, CD177, QPCT, SCAF4, SNRPD3, BCL9L, THBS1, SLC22A18AS, ARCN1, DHX16, SATB1, ST6GAL1, CXCL1, TDRD9, ZNF831, MTCH1, FAM86HP, DHX8, RNF114, and DCTN4. In some embodiments, the plurality of disease-associated genomic loci comprises the genes BCAT1, USP32P2, CD177, QPCT, SCAF4, SNRPD3, BCL9L, THBS1, SLC22A18AS, ARCN1, DHX16, SATB1, ST6GAL1, CXCL1, TDRD9, ZNF831, MTCH1, FAM86HP, DHX8, RNF114, and DCTN4. In some embodiments, the plurality of disease-associated genomic loci consists of the genes BCAT1, USP32P2, CD177, QPCT, SCAF4, SNRPD3, BCL9L, THBS1, SLC22A18AS, ARCN1, DHX16, SATB1, ST6GAL1, CXCL1, TDRD9, ZNF831, MTCH1, FAM86HP, DHX8, RNF114, and DCTN4

In some embodiments, the one or more clinical characteristics includes, 1, 2, 3, 4, 5, 6, 7 or 8 clinical characteristics selected from the group of clinical characteristics listed in Table 6. In some embodiments, the one or more clinical characteristics includes size of the nodule. In some embodiments, the one or more clinical characteristics includes age of the subject. In some embodiments, the one or more clinical characteristics includes presence of the nodule in the lung upper lobe. In some embodiments, the one or more clinical characteristics includes size of the nodule, age of the subject, presence of the nodule in the lung upper lobe, or any combination thereof.

In some embodiments, the plurality of disease-associated genomic loci comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7, and the one or more clinical characteristics comprises 1, 2, 3, 4, 5, 6, 7 or 8 clinical characteristics selected from the group of clinical characteristics listed in Table 6. In some embodiments, the plurality of disease-associated genomic loci comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7, and the one or more clinical characteristics of the subject comprises size of the nodule, age of the patient, presence of the nodule in the lung upper lobe, or any combination thereof. In some embodiments, the plurality of disease-associated genomic loci comprise the 31 genes listed in Table 7, and the one or more clinical characteristics comprise the size of the nodule, age of the patient, and the presence of the nodule in the lung upper lobe. In some embodiments, the plurality of disease-associated genomic loci consist of the 31 genes listed in Table 7, and the one or more clinical characteristics consist of the size of the nodule, age of the patient, and the presence of the nodule in the lung upper lobe.

In some embodiments, the method comprises classifying the lung nodule of the subject as the malignant lung nodule or the benign lung nodule with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In some embodiments, the method comprises classifying the lung nodule of the subject as the malignant lung nodule or the benign lung nodule with an accuracy of about 80% to about 100%. In some embodiments, the method comprises classifying the lung nodule of the subject as the malignant lung nodule or the benign lung nodule with an accuracy of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. In some embodiments, the method comprises classifying the lung nodule of the subject as the malignant lung nodule or the benign lung nodule with an accuracy of about 80%, about 85%, about 90%, about 92%, about 94%, about 95%, about 96%, about 97%, about 98%, about 99%, about 99.5%, or about 100%. In some embodiments, the method comprises classifying the lung nodule of the subject as the malignant lung nodule or the benign lung nodule with an accuracy of at least about 80%, about 85%, about 90%, about 92%, about 94%, about 95%, about 96%, about 97%, about 98%, about 99%, or about 99.5%. In some embodiments, the method comprises classifying the lung nodule of the subject as the malignant lung nodule or the benign lung nodule with an accuracy of at most about 85%, about 90%, about 92%, about 94%, about 95%, about 96%, about 97%, about 98%, about 99%, about 99.5%, or about 100%.

In some embodiments, the method comprises classifying the lung nodule of the subject as the malignant lung nodule or the benign lung nodule with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In some embodiments, the method comprises classifying the lung nodule of the subject as the malignant lung nodule or the benign lung nodule with a sensitivity of about 80% to about 100%. In some embodiments, the method comprises classifying the lung nodule of the subject as the malignant lung nodule or the benign lung nodule with a sensitivity of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. In some embodiments, the method comprises classifying the lung nodule of the subject as the malignant lung nodule or the benign lung nodule with a sensitivity of about 80%, about 85%, about 90%, about 92%, about 94%, about 95%, about 96%, about 97%, about 98%, about 99%, about 99.5%, or about 100%. In some embodiments, the method comprises classifying the lung nodule of the subject as the malignant lung nodule or the benign lung nodule with a sensitivity of at least about 80%, about 85%, about 90%, about 92%, about 94%, about 95%, about 96%, about 97%, about 98%, about 99%, or about 99.5%. In some embodiments, the method comprises classifying the lung nodule of the subject as the malignant lung nodule or the benign lung nodule with a sensitivity of at most about 85%, about 90%, about 92%, about 94%, about 95%, about 96%, about 97%, about 98%, about 99%, about 99.5%, or about 100%.

In some embodiments, the method comprises classifying the lung nodule of the subject as the malignant lung nodule or the benign lung nodule with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In some embodiments, the method comprises classifying the lung nodule of the subject as the malignant lung nodule or the benign lung nodule with a specificity of about 80% to about 100%. In some embodiments, the method comprises classifying the lung nodule of the subject as the malignant lung nodule or the benign lung nodule with a specificity of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. In some embodiments, the method comprises classifying the lung nodule of the subject as the malignant lung nodule or the benign lung nodule with a specificity of about 80%, about 85%, about 90%, about 92%, about 94%, about 95%, about 96%, about 97%, about 98%, about 99%, about 99.5%, or about 100%. In some embodiments, the method comprises classifying the lung nodule of the subject as the malignant lung nodule or the benign lung nodule with a specificity of at least about 80%, about 85%, about 90%, about 92%, about 94%, about 95%, about 96%, about 97%, about 98%, about 99%, or about 99.5%. In some embodiments, the method comprises classifying the lung nodule of the subject as the malignant lung nodule or the benign lung nodule with a specificity of at most about 85%, about 90%, about 92%, about 94%, about 95%, about 96%, about 97%, about 98%, about 99%, about 99.5%, or about 100%.

In some embodiments, the method comprises classifying the lung nodule of the subject as the malignant lung nodule or the benign lung nodule with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In some embodiments, the method comprises classifying the lung nodule of the subject as the malignant lung nodule or the benign lung nodule with a positive predictive value of about 80% to about 100%. In some embodiments, the method comprises classifying the lung nodule of the subject as the malignant lung nodule or the benign lung nodule with a positive predictive value of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. In some embodiments, the method comprises classifying the lung nodule of the subject as the malignant lung nodule or the benign lung nodule with a positive predictive value of about 80%, about 85%, about 90%, about 92%, about 94%, about 95%, about 96%, about 97%, about 98%, about 99%, about 99.5%, or about 100%. In some embodiments, the method comprises classifying the lung nodule of the subject as the malignant lung nodule or the benign lung nodule with a positive predictive value of at least about 80%, about 85%, about 90%, about 92%, about 94%, about 95%, about 96%, about 97%, about 98%, about 99%, or about 99.5%. In some embodiments, the method comprises classifying the lung nodule of the subject as the malignant lung nodule or the benign lung nodule with a positive predictive value of at most about 85%, about 90%, about 92%, about 94%, about 95%, about 96%, about 97%, about 98%, about 99%, about 99.5%, or about 100%.

In some embodiments, the method comprises classifying the lung nodule of the subject as the malignant lung nodule or the benign lung nodule with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In some embodiments, the method comprises classifying the lung nodule of the subject as the malignant lung nodule or the benign lung nodule with a negative predictive value of about 80% to about 100%. In some embodiments, the method comprises classifying the lung nodule of the subject as the malignant lung nodule or the benign lung nodule with a negative predictive value of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. In some embodiments, the method comprises classifying the lung nodule of the subject as the malignant lung nodule or the benign lung nodule with a negative predictive value of about 80%, about 85%, about 90%, about 92%, about 94%, about 95%, about 96%, about 97%, about 98%, about 99%, about 99.5%, or about 100%. In some embodiments, the method comprises classifying the lung nodule of the subject as the malignant lung nodule or the benign lung nodule with a negative predictive value of at least about 80%, about 85%, about 90%, about 92%, about 94%, about 95%, about 96%, about 97%, about 98%, about 99%, or about 99.5%. In some embodiments, the method comprises classifying the lung nodule of the subject as the malignant lung nodule or the benign lung nodule with a negative predictive value of at most about 85%, about 90%, about 92%, about 94%, about 95%, about 96%, about 97%, about 98%, about 99%, about 99.5%, or about 100%.

In some embodiments, the method comprises classifying the lung nodule of the subject as the malignant lung nodule or the benign lung nodule with an Area-Under-Curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99. The lung nodule of the subject can be classified as the malignant lung nodule or the benign lung nodule with a machine learning model having a receiver operating characteristic (ROC) curve with an AUC of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99. The lung nodule of the subject can be classified as the malignant lung nodule or the benign lung nodule with a machine learning model having a ROC curve with an AUC of about 0.8 to about 1. The lung nodule of the subject can be classified as the malignant lung nodule or the benign lung nodule with a machine learning model having a ROC curve with an AUC of about 0.8 to about 0.85, about 0.8 to about 0.9, about 0.8 to about 0.92, about 0.8 to about 0.94, about 0.8 to about 0.95, about 0.8 to about 0.96, about 0.8 to about 0.97, about 0.8 to about 0.98, about 0.8 to about 0.99, about 0.8 to about 0.995, about 0.8 to about 1, about 0.85 to about 0.9, about 0.85 to about 0.92, about 0.85 to about 0.94, about 0.85 to about 0.95, about 0.85 to about 0.96, about 0.85 to about 0.97, about 0.85 to about 0.98, about 0.85 to about 0.99, about 0.85 to about 0.995, about 0.85 to about 1, about 0.9 to about 0.92, about 0.9 to about 0.94, about 0.9 to about 0.95, about 0.9 to about 0.96, about 0.9 to about 0.97, about 0.9 to about 0.98, about 0.9 to about 0.99, about 0.9 to about 0.995, about 0.9 to about 1, about 0.92 to about 0.94, about 0.92 to about 0.95, about 0.92 to about 0.96, about 0.92 to about 0.97, about 0.92 to about 0.98, about 0.92 to about 0.99, about 0.92 to about 0.995, about 0.92 to about 1, about 0.94 to about 0.95, about 0.94 to about 0.96, about 0.94 to about 0.97, about 0.94 to about 0.98, about 0.94 to about 0.99, about 0.94 to about 0.995, about 0.94 to about 1, about 0.95 to about 0.96, about 0.95 to about 0.97, about 0.95 to about 0.98, about 0.95 to about 0.99, about 0.95 to about 0.995, about 0.95 to about 1, about 0.96 to about 0.97, about 0.96 to about 0.98, about 0.96 to about 0.99, about 0.96 to about 0.995, about 0.96 to about 1, about 0.97 to about 0.98, about 0.97 to about 0.99, about 0.97 to about 0.995, about 0.97 to about 1, about 0.98 to about 0.99, about 0.98 to about 0.995, about 0.98 to about 1, about 0.99 to about 0.995, about 0.99 to about 1, or about 0.995 to about 1. The lung nodule of the subject can be classified as the malignant lung nodule or the benign lung nodule with a machine learning model having a ROC curve with an AUC of about 0.8, about 0.85, about 0.9, about 0.92, about 0.94, about 0.95, about 0.96, about 0.97, about 0.98, about 0.99, about 0.995, or about 1. The lung nodule of the subject can be classified as the malignant lung nodule or the benign lung nodule with a machine learning model having a ROC curve with an AUC of at least about 0.8, about 0.85, about 0.9, about 0.92, about 0.94, about 0.95, about 0.96, about 0.97, about 0.98, about 0.99, or about 0.995. The lung nodule of the subject can be classified as the malignant lung nodule or the benign lung nodule with a machine learning model having a ROC curve with an AUC of at most about 0.85, about 0.9, about 0.92, about 0.94, about 0.95, about 0.96, about 0.97, about 0.98, about 0.99, about 0.995, or about 1.

In some embodiments, the subject has a lung cancer. In some embodiments, the subject is suspected of having a lung cancer. In some embodiments, the subject is at elevated risk of having a lung cancer. In some embodiments, the subject is asymptomatic for a lung cancer.

In certain embodiments, the method comprises optionally performing biopsy of the lung nodule of the subject based at least in part on the classification of the lung nodule of the subject as the malignant lung nodule or the benign lung nodule. In certain embodiments, the method comprises optionally performing biopsy of the lung nodule of the subject based at least in part on the classification of the lung nodule of the subject as the malignant lung nodule. In certain embodiments, biopsy of the lung nodule is not performed. In some embodiments, the method further contains administering a treatment to the subject based at least in part on the classification of the lung nodule of the subject as the malignant lung nodule or the benign lung nodule. In some embodiments, the method contains administering a treatment to the subject based at least in part on the classification of the lung nodule of the subject as the malignant lung nodule. In some embodiments, the treatment is configured to treat a lung cancer of the subject. In some embodiments, the treatment is configured to reduce a severity of a lung cancer of the subject. In some embodiments, the treatment is configured to reduce a risk of having a lung cancer of the subject. The treatment can include one or more treatments of lung cancer. In some embodiments, the treatment is selected from the group consisting of: surgery, chemotherapy, targeted therapy, immunotherapy, radiotherapy, and any combination thereof.

In some embodiments, (b) comprises comparing the data set to a reference data set. In some embodiments, the reference data set comprises gene expression measurements of reference biological samples from each of the plurality of lung disease-associated genomic loci, and optionally clinical characteristics data of the one or more clinical characteristics of reference subjects. In some embodiments, the reference biological samples comprise a first plurality of biological samples obtained or derived from reference subjects having a malignant lung nodule and a second plurality of biological samples obtained or derived from reference subjects having a benign lung nodule.

In some embodiments, (b) comprises using a trained machine learning classifier to analyze the data set to classify the lung nodule of the subject as the malignant lung nodule or the benign lung nodule. The trained machine-learning classifier can generate an inference of whether the data set is indicative of a malignant lung nodule or a benign lung nodule. In some embodiments, the trained machine learning classifier is trained using gene expression data obtained by a data analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, and a Gene Set Variation Analysis (GSVA) tool (e.g., P-Scope).

In some embodiments, the trained machine learning classifier is a supervised machine learning algorithm or an unsupervised machine learning algorithm. In some embodiments, the trained machine learning classifier is selected from the group consisting of a linear regression, a logistic regression (LOG), a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB), a neural network, a Random Forest (RF), a deep learning algorithm, a linear discriminant analysis (LDA), a decision tree learning (DTREE), an adaptive boosting (ADB) and any combination thereof. In some embodiments, the trained machine learning classifier comprises the LOG. In some embodiments, the trained machine learning classifier comprises the Ridge regression. In some embodiments, the trained machine learning classifier comprises the Lasso regression. In some embodiments, the trained machine learning classifier comprises the GLM. In some embodiments, the trained machine learning classifier comprises the kNN. In some embodiments, the trained machine learning classifier comprises the SVM. In some embodiments, the trained machine learning classifier comprises the GBM. In some embodiments, the trained machine learning classifier comprises the RF. In some embodiments, the trained machine learning classifier comprises the NB. In some embodiments, the trained machine learning classifier comprises the EN regression. In some embodiments, the trained machine learning classifier comprises the neural network. In some embodiments, the trained machine learning classifier comprises the deep learning algorithm. In some embodiments, the trained machine learning classifier comprises the LDA. In some embodiments, the trained machine learning classifier comprises the DTREE. In some embodiments, the trained machine learning classifier comprises the ADB. In certain embodiments, oversampling or undersampling correction is made during training of the machine learning model.

In some embodiments, the method includes receiving, as an output of the machine-learning classifier, the inference indicating whether the data set is indicative of the malignant lung nodule or the benign lung nodule.

In some embodiments, the biological sample is selected from the group consisting of: a blood sample, isolated peripheral blood mononuclear cells (PBMCs), a lung biopsy sample, nasal fluid, saliva, and any derivative thereof. In some embodiments, the biological sample is a blood sample, or any derivative thereof. In some embodiments, the biological sample is isolated peripheral blood mononuclear cells, (PBMCs) or any derivative thereof. In some embodiments, the biological sample is a lung biopsy sample, or any derivative thereof. In some embodiments, the biological sample is a nasal fluid sample, or derivative thereof. In some embodiments, the biological sample is a saliva sample, or any derivative thereof.

In some embodiments, the method further comprises determining a likelihood of the classification of the lung nodule of the subject as the malignant lung nodule or the benign lung nodule. In some embodiments, the likelihood is about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100%. In some embodiments, the likelihood is least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises monitoring the lung nodule of the subject, wherein the monitoring comprises assessing the lung nodule of the subject at a plurality of time points. In some embodiments, a difference in the assessment of the lung nodule of the subject among the plurality of time points is indicative of one or more clinical indications selected from the group consisting of: (i) a diagnosis of the lung nodule of the subject, (ii) a prognosis of the lung nodule of the subject, and (iii) an efficacy or non-efficacy of a course of treatment for treating the lung nodule of the subject. In some embodiments, the plurality of time points comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 different time points.

In an aspect, the present disclosure provides a method for assessing a lung nodule of a patient. The method can include, any one of, any combination of, or all of steps a′, b′, c′ and d′. Step a′ can include obtaining a data set containing gene expression measurements of a biological sample obtained or derived from the patient, of at least two lung disease-associated genes. The data set can be obtained by assaying the biological sample. In some embodiments, the at least two lung disease-associated genes are selected from the group of genes listed in Table 4. In some embodiments, the at least two lung disease-associated genes are selected from the group of genes listed in Table 1. In some embodiments, the at least two lung disease-associated genes are selected from the group of genes listed in Table 2. In some embodiments, the at least two lung disease-associated genes are selected from the group of genes listed in Table 3. In some embodiments, the at least two lung disease-associated genes are selected from the group of genes listed in Table 5. In some embodiments, the at least two lung disease-associated genes are selected from the group of genes listed in Table 7. In some embodiments, the at least two lung disease-associated genes are selected from the group of genes listed in Table 8. Step b′ can include providing the data set as an input to a machine-learning model trained to generate an inference of whether the data set is indicative of a malignant lung nodule or a benign lung nodule. Step c′ can include receiving, as an output of the machine-learning model, the inference indicating whether the data set is indicative of the malignant lung nodule or the benign lung nodule. Step d′ can include electronically outputting a report classifying the lung nodule of the patient as the malignant lung nodule or the benign lung nodule. In some embodiments, the data set of step a′, can further include clinical characteristics data of one or more clinical characteristics of the patient. In some embodiments, the one or more clinical characteristics are selected from the group of clinical characteristics listed in Table 6. In some embodiments, the data set of step a′, contains i) gene expression measurements of the biological sample of at least two lung disease-associated genes selected from the group of genes listed in in any one or more of Tables 1, 2, 3, 4, 5, 7, and 8, and ii) optionally clinical characteristics data of one or more clinical characteristics of the patient selected from the group of clinical characteristics listed in Table 6. The gene expression measurement of the biological sample can be performed using any suitable technique, such as any suitable RNA quantification technique, including but not limited to RNA-seq, Ampli-seq, and the like.

In some embodiments, the at least two lung disease-associated genes, e.g. as of step a′, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, or 182, or any value or range there between, genes selected from the group of genes listed in Table 1. In some embodiments, the at least two lung disease-associated genes, e.g. as of step a′, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, or 175, or any value or range there between, genes selected from the group of genes listed in Table 2. In some embodiments, the at least two lung disease-associated genes, e.g. as of step a′, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 or 62, or any value or range there between, genes selected from the group of genes listed in Table 3. In some embodiments, the at least two lung disease-associated genes, e.g. as of step a′, include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295, or any value or range there between, genes selected from the group of genes listed in Table 4. In some embodiments, the at least two lung disease-associated genes, e.g. as of step a′, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, or 142, or any value or range there between, genes selected from the group of genes listed in Table 5. In some embodiments, the at least two lung disease-associated genes, e.g. as of step a′, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7. In some embodiments, the at least two lung disease-associated genes of step a′, are selected from BCAT1, CRCP, COA4, OVCA2, POM121, HLA-DPA1, VPS37C, MGST2, RNF220, HDAC3, NFE2L1, WDR20, CNPY4, HOXB2, C6orf120, TMEM8A, ASAP1-IT2, C15orf54, CD101, FNBP1, TECR, PROK2, SLC35B3, TDRD9, CLHC1, LPL, IFITM3, OGFOD3, EIF2B3, TMEM65, and MKRN3. In some embodiments, the at least two lung disease-associated genes, e.g. as of step a′, include at least 2, 3, 4, 5, 6, 7, 8, 9, 10, 11, 12, 13, 14, 15, 16, 17, 18, 19, 20, or 21 genes selected from the group of genes listed in Table 8. In some embodiments, the at least two lung disease-associated genes of step a′, are selected from BCAT1, USP32P2, CD177, QPCT, SCAF4, SNRPD3, BCL9L, THBS1, SLC22A18AS, ARCN1, DHX16, SATB1, ST6GAL1, CXCL1, TDRD9, ZNF831, MTCH1, FAM86HP, DHX8, RNF114, and DCTN4. In some embodiments, the one or more clinical characteristics includes, 1, 2, 3, 4, 5, 6, 7 or 8 clinical characteristics selected from the group of clinical characteristics listed in Table 6. In some embodiments, the one or more clinical characteristics includes size of the nodule. In some embodiments, the one or more clinical characteristics includes age of the patient. In some embodiments, the one or more clinical characteristics includes presence of the nodule in the lung upper lobe. In some embodiments, the one or more clinical characteristics of the patient includes size of the nodule, age of the patient, presence of the nodule in the lung upper lobe, or any combination thereof. In some embodiments, the at least two lung disease-associated genes of step a′, comprise the 31 genes listed in Table 7, and the one or more clinical characteristics of step a′, comprise the size of the nodule, age of the patient, and the presence of the nodule in the lung upper lobe. In some embodiments, the at least two lung disease-associated genes of step a′, consist of the 31 genes listed in Table 7, and the one or more clinical characteristics of step a′, consist of the size of the nodule, age of the patient, and the presence of the nodule in the lung upper lobe. In some embodiments, the data set of step a′, contains i) gene expression measurements of the biological sample of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease-associated genes selected from the group of genes listed in Table 7, and ii) clinical characteristics data of 1, 2, 3, 4, 5, 6, 7 or 8 clinical characteristics of the patient selected from the group of clinical characteristics listed in Table 6. In some embodiments, the data set of step a′, contains i) gene expression measurements of the biological sample of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease-associated genes selected from the group of genes listed in Table 7, and ii) clinical characteristics data of the clinical characteristics of the patient selected from size of the nodule, age of the patient, presence of the nodule in the lung upper lobe, or any combination thereof.

In some embodiments, the biological sample is a blood sample, isolated peripheral blood mononuclear cells (PBMCs), a lung biopsy sample, nasal fluid, saliva, or any derivative thereof. In some embodiments, the biological sample is a blood sample or any derivative thereof. In some embodiments, the biological sample is isolated peripheral blood mononuclear cells (PBMCs) or any derivative thereof. In some embodiments, the biological sample is a lung biopsy sample, or any derivative thereof. In some embodiments, the biological sample is a nasal fluid sample, or any derivative thereof. In some embodiments, the biological sample is a saliva sample, or any derivative thereof.

The method can classify the lung nodule of the patient as the malignant lung nodule or the benign lung nodule with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model, e.g. of step b′, can infer whether the data set is indicative of a malignant lung nodule or a benign lung nodule with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The method can classify the lung nodule of the patient as the malignant lung nodule or the benign lung nodule with an accuracy of about 80% to about 100%. The method can classify the lung nodule of the patient as the malignant lung nodule or the benign lung nodule with an accuracy of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The method can classify the lung nodule of the patient as the malignant lung nodule or the benign lung nodule with an accuracy of about 80%, about 85%, about 90%, about 92%, about 94%, about 95%, about 96%, about 97%, about 98%, about 99%, about 99.5%, or about 100%. The method can classify the lung nodule of the patient as the malignant lung nodule or the benign lung nodule with an accuracy of at least about 80%, about 85%, about 90%, about 92%, about 94%, about 95%, about 96%, about 97%, about 98%, about 99%, or about 99.5%. The method can classify the lung nodule of the patient as the malignant lung nodule or the benign lung nodule with an accuracy of at most about 85%, about 90%, about 92%, about 94%, about 95%, about 96%, about 97%, about 98%, about 99%, about 99.5%, or about 100%.

The method can classify the lung nodule of the patient as the malignant lung nodule or the benign lung nodule with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model, e.g. of step b′, can infer whether the data set is indicative of a malignant lung nodule or a benign lung nodule with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The method can classify the lung nodule of the patient as the malignant lung nodule or the benign lung nodule with a sensitivity of about 80% to about 100%. The method can classify the lung nodule of the patient as the malignant lung nodule or the benign lung nodule with a sensitivity of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The method can classify the lung nodule of the patient as the malignant lung nodule or the benign lung nodule with a sensitivity of about 80%, about 85%, about 90%, about 92%, about 94%, about 95%, about 96%, about 97%, about 98%, about 99%, about 99.5%, or about 100%. The method can classify the lung nodule of the patient as the malignant lung nodule or the benign lung nodule with a sensitivity of at least about 80%, about 85%, about 90%, about 92%, about 94%, about 95%, about 96%, about 97%, about 98%, about 99%, or about 99.5%. The method can classify the lung nodule of the patient as the malignant lung nodule or the benign lung nodule with a sensitivity of at most about 85%, about 90%, about 92%, about 94%, about 95%, about 96%, about 97%, about 98%, about 99%, about 99.5%, or about 100%.

The method can classify the lung nodule of the patient as the malignant lung nodule or the benign lung nodule with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model, e.g. of step b′, can infer whether the data set is indicative of a malignant lung nodule or a benign lung nodule with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The method can classify the lung nodule of the patient as the malignant lung nodule or the benign lung nodule with a specificity of about 80% to about 100%. The method can classify the lung nodule of the patient as the malignant lung nodule or the benign lung nodule with a specificity of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The method can classify the lung nodule of the patient as the malignant lung nodule or the benign lung nodule with a specificity of about 80%, about 85%, about 90%, about 92%, about 94%, about 95%, about 96%, about 97%, about 98%, about 99%, about 99.5%, or about 100%. The method can classify the lung nodule of the patient as the malignant lung nodule or the benign lung nodule with a specificity of at least about 80%, about 85%, about 90%, about 92%, about 94%, about 95%, about 96%, about 97%, about 98%, about 99%, or about 99.5%. The method can classify the lung nodule of the patient as the malignant lung nodule or the benign lung nodule with a specificity of at most about 85%, about 90%, about 92%, about 94%, about 95%, about 96%, about 97%, about 98%, about 99%, about 99.5%, or about 100%.

The method can classify the lung nodule of the patient as the malignant lung nodule or the benign lung nodule with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model, e.g. of step b′, can infer whether the data set is indicative of a malignant lung nodule or a benign lung nodule with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The method can classify the lung nodule of the patient as the malignant lung nodule or the benign lung nodule with a positive predictive value of about 80% to about 100%. The method can classify the lung nodule of the patient as the malignant lung nodule or the benign lung nodule with a positive predictive value of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The method can classify the lung nodule of the patient as the malignant lung nodule or the benign lung nodule with a positive predictive value of about 80%, about 85%, about 90%, about 92%, about 94%, about 95%, about 96%, about 97%, about 98%, about 99%, about 99.5%, or about 100%. The method can classify the lung nodule of the patient as the malignant lung nodule or the benign lung nodule with a positive predictive value of at least about 80%, about 85%, about 90%, about 92%, about 94%, about 95%, about 96%, about 97%, about 98%, about 99%, or about 99.5%. The method can classify the lung nodule of the patient as the malignant lung nodule or the benign lung nodule with a positive predictive value of at most about 85%, about 90%, about 92%, about 94%, about 95%, about 96%, about 97%, about 98%, about 99%, about 99.5%, or about 100%.

The method can classify the lung nodule of the patient as the malignant lung nodule or the benign lung nodule with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model, e.g. of step b′, can infer whether the data set is indicative of a malignant lung nodule or a benign lung nodule with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The method can classify the lung nodule of the patient as the malignant lung nodule or the benign lung nodule with a negative predictive value of about 80% to about 100%. The method can classify the lung nodule of the patient as the malignant lung nodule or the benign lung nodule with a negative predictive value of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The method can classify the lung nodule of the patient as the malignant lung nodule or the benign lung nodule with a negative predictive value of about 80%, about 85%, about 90%, about 92%, about 94%, about 95%, about 96%, about 97%, about 98%, about 99%, about 99.5%, or about 100%. The method can classify the lung nodule of the patient as the malignant lung nodule or the benign lung nodule with a negative predictive value of at least about 80%, about 85%, about 90%, about 92%, about 94%, about 95%, about 96%, about 97%, about 98%, about 99%, or about 99.5%. The method can classify the lung nodule of the patient as the malignant lung nodule or the benign lung nodule with a negative predictive value of at most about 85%, about 90%, about 92%, about 94%, about 95%, about 96%, about 97%, about 98%, about 99%, about 99.5%, or about 100%.

The machine learning model, e.g. of step b′, can infer whether the data set is indicative of a malignant lung nodule or a benign lung nodule with a receiver operating characteristic (ROC) curve with an AUC of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99. The machine learning model, e.g. of step b′, can infer whether the data set is indicative of a malignant lung nodule or a benign lung nodule with a ROC curve with an AUC of about 0.8 to about 1. The machine learning model, e.g. of step b′, can infer whether the data set is indicative of a malignant lung nodule or a benign lung nodule with a ROC curve with an AUC of about 0.8 to about 0.85, about 0.8 to about 0.9, about 0.8 to about 0.92, about 0.8 to about 0.94, about 0.8 to about 0.95, about 0.8 to about 0.96, about 0.8 to about 0.97, about 0.8 to about 0.98, about 0.8 to about 0.99, about 0.8 to about 0.995, about 0.8 to about 1, about 0.85 to about 0.9, about 0.85 to about 0.92, about 0.85 to about 0.94, about 0.85 to about 0.95, about 0.85 to about 0.96, about 0.85 to about 0.97, about 0.85 to about 0.98, about 0.85 to about 0.99, about 0.85 to about 0.995, about 0.85 to about 1, about 0.9 to about 0.92, about 0.9 to about 0.94, about 0.9 to about 0.95, about 0.9 to about 0.96, about 0.9 to about 0.97, about 0.9 to about 0.98, about 0.9 to about 0.99, about 0.9 to about 0.995, about 0.9 to about 1, about 0.92 to about 0.94, about 0.92 to about 0.95, about 0.92 to about 0.96, about 0.92 to about 0.97, about 0.92 to about 0.98, about 0.92 to about 0.99, about 0.92 to about 0.995, about 0.92 to about 1, about 0.94 to about 0.95, about 0.94 to about 0.96, about 0.94 to about 0.97, about 0.94 to about 0.98, about 0.94 to about 0.99, about 0.94 to about 0.995, about 0.94 to about 1, about 0.95 to about 0.96, about 0.95 to about 0.97, about 0.95 to about 0.98, about 0.95 to about 0.99, about 0.95 to about 0.995, about 0.95 to about 1, about 0.96 to about 0.97, about 0.96 to about 0.98, about 0.96 to about 0.99, about 0.96 to about 0.995, about 0.96 to about 1, about 0.97 to about 0.98, about 0.97 to about 0.99, about 0.97 to about 0.995, about 0.97 to about 1, about 0.98 to about 0.99, about 0.98 to about 0.995, about 0.98 to about 1, about 0.99 to about 0.995, about 0.99 to about 1, or about 0.995 to about 1. The machine learning model, e.g. of step b′, can infer whether the data set is indicative of a malignant lung nodule or a benign lung nodule with a ROC curve with an AUC of about 0.8, about 0.85, about 0.9, about 0.92, about 0.94, about 0.95, about 0.96, about 0.97, about 0.98, about 0.99, about 0.995, or about 1. The machine learning model, e.g. of step b′, can infer whether the data set is indicative of a malignant lung nodule or a benign lung nodule with a ROC curve with an AUC of at least about 0.8, about 0.85, about 0.9, about 0.92, about 0.94, about 0.95, about 0.96, about 0.97, about 0.98, about 0.99, or about 0.995. The machine learning model, e.g. of step b′, can infer whether the data set is indicative of a malignant lung nodule or a benign lung nodule with a ROC curve with an AUC of at most about 0.85, about 0.9, about 0.92, about 0.94, about 0.95, about 0.96, about 0.97, about 0.98, about 0.99, about 0.995, or about 1.

The inference from the machine learning model can include a confidence value between 0 and 1, such as, 0, 0.1, 0.2, 0.3, 0.4, 0.5, 0.6, 0.7, 0.8, 0.9 or 1, or any value or range there between, that the nodule is malignant. Higher confidence values may be correlated with a higher likelihood that the nodule is malignant. A malignant nodule may be characterized by the ability to metastasize or grow invasively, which may be in contrast to benign nodules.

In some embodiments, the patient has a lung cancer. In some embodiments, the patient does not have lung cancer. In some embodiments, the patient is suspected of having a lung cancer. In some embodiments, the patient is at an elevated risk of having a lung cancer. In some embodiments, the patient is asymptomatic for a lung cancer.

In certain embodiments, the method includes optionally performing biopsy of the lung nodule of the patient based at least in part on the classification of the lung nodule of the patient as the malignant lung nodule or the benign lung nodule. In certain embodiments, the method includes optionally performing a biopsy of the lung nodule of the patient based at least in part on the classification of the lung nodule of the patient as the malignant lung nodule. In some embodiments, a biopsy is performed. In some embodiments, a biopsy is not performed. The decision to perform a biopsy may be made by one of skill in the art, based on knowledge and experience, in view of the classification of the lung nodule of the patient as the malignant lung nodule or the benign lung nodule. The decision to perform a biopsy may depend in part on the confidence value of the inference. In some embodiments, the method further comprises administering a treatment to the patient based at least in part on the classification of the lung nodule of the patient as the malignant lung nodule or the benign lung nodule. In some embodiments, the method comprises administering a treatment to the patient based at least in part on the classification of the lung nodule of the patient as the malignant lung nodule. In some embodiments, the treatment is configured to treat a lung cancer of the patient. In some embodiments, the treatment is configured to reduce a severity of a lung cancer of the patient. In some embodiments, the treatment is configured to reduce a risk of having a lung cancer of the patient. The treatment can include one or more treatments of lung cancer. In some embodiments, the treatment is surgery, chemotherapy, targeted therapy, immunotherapy, radiotherapy, or any combination thereof.

The trained machine-learning model, e.g. of step b′, can generate the inference of whether the data set is indicative of a malignant lung nodule or a benign lung nodule, by comparing the data set to a reference data set. The machine-learning model can be trained using the reference data set. In some embodiments, the reference data set contains gene expression measurements of a plurality of genes of a plurality of reference biological samples from a plurality of reference subjects having lung nodule; data regarding whether the lung nodules of the reference subjects are benign or malignant; and optionally clinical characteristics data of the reference subjects of one or more clinical characteristics selected from the group of clinical characteristics listed in Table 6. A first portion of the plurality of reference subjects can have benign lung nodule, and a second portion of the plurality of reference subjects can have malignant lung nodule. In some embodiments, the reference data set contains a plurality of individual reference data sets. A respective individual reference data set of the plurality of individual reference data sets can contain i) gene expression measurements of a plurality of genes of a reference biological sample from a reference subject having a lung nodule, ii) data regarding whether the lung nodule of the reference subject is benign or malignant, and iii) optionally clinical characteristics data of one or more clinical characteristics selected from the group of clinical characteristics listed in Table 6, of the reference subject. The plurality of individual reference data sets can be obtained from the plurality of reference subjects. In some embodiments, different individual reference data sets are obtained from different reference subjects. In some embodiments, each of the individual reference data set contains i) gene expression measurements of the plurality of genes of a reference biological sample from one reference subject, ii) data regarding whether the lung nodule of the one reference subject is benign or malignant, and iii) optionally clinical characteristics data of the one or more clinical characteristics selected from the group of clinical characteristics listed in Table 6 of the one reference subject, wherein different individual reference data sets are obtained from different reference subjects. In certain embodiments, oversampling or undersampling correction is made during training of the machine learning model. For example, if a reference data set includes a greater number of samples identified as benign and a relatively fewer number of samples identified as malignant, the malignant samples may be oversampled to produce a data set that has equal number of benign and malignant samples. The plurality of genes of the reference data set can include at least 2 genes selected from the group of genes listed in in any one or more of Tables 1, 2, 3, 4, 5, 7, and 8. In some embodiments, the plurality of genes of the reference data set include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, or 182, or any value or range there between, genes selected from the group of genes listed in Table 1. In some embodiments, the plurality of genes of the reference data set include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, or 175, or any value or range there between, genes selected from the group of genes listed in Table 2. In some embodiments, the plurality of genes of the reference data set include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 or 62, or any value or range there between, genes selected from the group of genes listed in Table 3. In some embodiments, the plurality of genes of the reference data set include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295, or any value or range there between, genes selected from the group of genes listed in Table 4. In some embodiments, the plurality of genes of the reference data set include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, or 142, or any value or range there between, genes selected from the group of genes listed in Table 5. In some embodiments, the plurality of genes of the reference data set include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7. In some embodiments, the plurality of genes of the reference data set include at least 2, 3, 4, 5, 6, 7, 8, 9, 10, 11, 12, 13, 14, 15, 16, 17, 18, 19, 20, or 21, genes selected from the group of genes listed in Table 8. In some embodiments, the one or more clinical characteristics of the reference data set include, 1, 2, 3, 4, 5, 6, 7 or 8 clinical characteristics selected from the group of clinical characteristics listed in Table 6. In some embodiments, the one or more clinical characteristics of the reference data set includes size of the nodule. In some embodiments, the one or more clinical characteristics of the reference data set includes age of the patient. In some embodiments, the one or more clinical characteristics of the reference data set includes presence of the nodule in the lung upper lobe. In some embodiments, the one or more clinical characteristics of the reference data set includes size of the nodule, age of the patient, presence of the nodule in the lung upper lobe, or any combination thereof. In some embodiments, the plurality of genes of the reference data set include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7, and the one or more clinical characteristics of the reference data set includes 1, 2, 3, 4, 5, 6, 7 or 8 clinical characteristics selected from the group of clinical characteristics listed in Table 6. In some embodiments, the plurality of genes of the reference data set include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7, and the one or more clinical characteristics of the reference data set include size of the nodule, age of the patient, presence of the nodule in the lung upper lobe, or any combination thereof. In some embodiments, the plurality of genes of the reference data set include comprise the 31 genes listed in Table 7, and the one or more clinical characteristics of the reference data set comprise the size of the nodule, age of the patient, and the presence of the nodule in the lung upper lobe. In some embodiments, the plurality of genes of the reference data set consist of the 31 genes listed in Table 7, and the one or more clinical characteristics of the reference data set consist of the size of the nodule, age of the patient, and the presence of the nodule in the lung upper lobe. The genes of the data set and genes of the reference data set can at least partially overlap and/or the optional clinical characteristics of the data set and optional clinical characteristics of the reference data set can at least partially overlap. In some embodiments, the reference biological sample is a blood sample, isolated peripheral blood mononuclear cells (PBMCs), a lung biopsy sample, nasal fluid, saliva, or any derivative thereof. In some embodiments, the reference biological sample is a blood sample or any derivative thereof. In some embodiments, the reference biological sample is isolated peripheral blood mononuclear cells (PBMCs) or any derivative thereof. In some embodiments, the reference biological sample is a lung biopsy sample, or any derivative thereof. In some embodiments, the reference biological sample is a nasal fluid sample, or any derivative thereof. In some embodiments, the reference biological sample is a saliva sample, or any derivative thereof. The reference subjects can be human.

Gene expression data can be obtained by a data analysis tool selected from the group: a BIG-C™ big data analysis tool, an I-Scope™ big data analysis tool, a T-Scope™ big data analysis tool, a Cell Scan big data analysis tool, an MS (Molecular Signature) Scoring™ analysis tool, and a Gene Set Variation Analysis (GSVA) tool (e.g., P-Scope).

In some embodiments, the trained machine learning model, e.g. of step b′, is a supervised machine learning algorithm or an unsupervised machine learning algorithm. In some embodiments, the trained machine learning model is trained using linear regression, logistic regression (LOG), Ridge regression, Lasso regression, elastic net (EN) regression, support vector machine (SVM), gradient boosted machine (GBM), k nearest neighbors (kNN), generalized linear model (GLM), naïve Bayes (NB), neural network, Random Forest (RF), deep learning algorithm, linear discriminant analysis (LDA), a decision tree learning (DTREE), adaptive boosting (ADB), or any combination thereof. In some embodiments, the trained machine learning model is trained using LOG. In some embodiments, the trained machine learning model is trained using Ridge regression. In some embodiments, the trained machine learning model is trained using Lasso regression. In some embodiments, the trained machine learning model is trained using GLM. In some embodiments, the trained machine learning model is trained using kNN. In some embodiments, the trained machine learning model is trained using SVM. In some embodiments, the trained machine learning model is trained using GBM. In some embodiments, the trained machine learning model is trained using RF. In some embodiments, the trained machine learning model is trained using NB. In some embodiments, the trained machine learning model is trained using the EN regression. In some embodiments, the trained machine learning model is trained using neural network. In some embodiments, the trained machine learning model is trained using deep learning algorithm. In some embodiments, the trained machine learning model is trained using LDA. In some embodiments, the trained machine learning model is trained using DTREE. In some embodiments, the trained machine learning model is trained using ADB.

In some embodiments, the method comprises determining a likelihood of the classification of the lung nodule of the patient as the malignant lung nodule or the benign lung nodule. In some embodiments, the likelihood is about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100%. In some embodiments, the likelihood is least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises monitoring the lung nodule of the patient, wherein the monitoring comprises assessing the lung nodule of the patient at a plurality of time points. In some embodiments, a difference in the assessment of the lung nodule of the patient among the plurality of time points is indicative of one or more clinical indications selected from the group consisting of: (i) a diagnosis of the lung nodule of the patient, (ii) a prognosis of the lung nodule of the patient, and (iii) an efficacy or non-efficacy of a course of treatment for treating the lung nodule of the patient. In some embodiments, the plurality of time points comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 different time points.

In another aspect, the present disclosure provides a method for determining a gene set capable of classifying a lung nodule, benign or malignant. Gene expression measurements of one or more genes of the gene set, of a biological sample (e.g. blood) from a subject can be used to classify a lung nodule of the subject, benign or malignant without performing biopsy of the nodule. In some embodiments, a biopsy of the nodule is performed to confirm and/or follow-up the classification results obtained by using the gene expression measurements data. In some embodiments, a biopsy of the nodule is not performed. The method can include any one of, any combination of, or all of steps a″, b″, c″ and d″. In step a″, a reference data set can be obtained and/or provided. The reference data set can contain a plurality of individual reference data sets. A respective individual reference data set of the plurality of individual reference data sets can contain i) gene expression measurements of a plurality of genes of a reference biological sample from a reference subject having a lung nodule, ii) data regarding whether the lung nodule is benign or malignant, and iii) optionally clinical characteristics data of one or more clinical characteristics selected from the group of clinical characteristics listed in Table 6 of the reference subject. The plurality of individual reference data sets can be obtained from a plurality of reference subjects. In some embodiments, different individual reference data sets are obtained from different reference subjects. In some embodiments, each of the individual reference data set contains i) gene expression measurements of the plurality of genes of a reference biological sample from one reference subject, ii) data regarding whether the lung nodule of the one reference subject is benign or malignant, and iii) optionally clinical characteristics data of the one or more clinical characteristics selected from the group of the clinical characteristics listed in Table 6 of the one reference subject, wherein different individual reference data sets are obtained from different reference subjects. A first portion of the plurality of reference subjects can have benign lung nodule, and a second portion of the plurality of reference subjects can have malignant lung nodule. The reference data set can contain gene expression measurements of the plurality of genes of a plurality of reference biological samples from the plurality of reference subjects having lung nodule; data regarding whether the lung nodules of the reference subjects are benign or malignant; and optionally clinical characteristics data of the reference subjects of one or more clinical characteristics selected from the group of clinical characteristics listed in Table 6. In step b″, a machine learning model can be trained using the reference data set to infer whether a lung nodule is benign or malignant based on at least in part on one or more predictors selected from i) the plurality of genes, and ii) optionally the one or more clinical characteristics. The trained machine learning model can infer whether the lung nodule from a subject is benign or malignant based on at least in part on the gene expression measurements of the plurality of genes from a biological sample of the subject, and optionally clinical characteristics data of the one or more clinical characteristics of the subject. In some embodiments, the machine learning model can be trained using a training data set containing a first portion of the reference data set, and a validation data set containing a second portion of the reference data set. In certain embodiments, oversampling or undersampling correction is made during training of the machine learning model. For example, if a data set includes a greater number of samples identified as benign and a relatively fewer number of samples identified as malignant, the malignant samples may be oversampled to produce a data set that has equal number of benign and malignant samples. In step c″, feature importance values of the plurality of genes can be determined. In step d″, the gene set can be selected. In some embodiments, the gene set is selected as predictors that are used to train the machine learning model. The gene set, may be selected based at least in part on the feature importance values. In some embodiments, the feature importance values of the genes of the gene set, are within top 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, or 250, or any value or range there between, feature importance values of the plurality of genes. In some embodiments, the feature importance of the genes of the gene set, have accuracy, greater than 30%, 35%, 40%, 45%, 50%, 55%, 60%, 65%, 70%, 75%, 80% or 90%. In some embodiments, the feature importance of the genes of the gene set, have threshold importance, greater than 30, 35, 40, 45, 50, 55, 60, 65, 70, 75, 80 or 90. In certain embodiments, the top 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, or 250, or any value or range there between, predictors of the machine learning model include the genes of the gene set. In some embodiments, the plurality of genes of the reference data set of step a″, include at least 2 genes selected from the group of genes listed in Table 9. In some embodiments, the plurality of genes of the reference data set of step a″, include at least 2 genes selected from the group of genes listed in Table 1. In some embodiments, the plurality of genes of the reference data set of step a″, include at least 2 genes selected from the group of genes listed in Table 2. In some embodiments, the plurality of genes of the reference data set of step a″, include at least 2 genes selected from the group of genes listed in Table 3. In some embodiments, the plurality of genes of the reference data set of step a″, include at least 2 genes selected from the group of genes listed in Table 4. In some embodiments, the plurality of genes of the reference data set of step a″, include at least 2 genes selected from the group of genes listed in Table 5. In some embodiments, the plurality of genes of the reference data set of step a″, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 300, 400, 500, 600, 700, 800, 900, 1000, 1100 or 1178, or any value or range there between, genes selected from the group of genes listed in Table 9. In some embodiments, the plurality of genes of the reference data set of step a″, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, or 182, or any value or range there between, genes selected from the group of genes listed in Table 1. In some embodiments, the plurality of genes of the reference data set of step a″, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, or 175, or any value or range there between, genes selected from the group of genes listed in Table 2. In some embodiments, the plurality of genes of the reference data set of step a″, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 or 62, or any value or range there between, genes selected from the group of genes listed in Table 3. In some embodiments, the plurality of genes of the reference data set of step a″, include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295, or any value or range there between, genes selected from the group of genes listed in Table 4. In some embodiments, the plurality of genes of the reference data set of step a″, include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, or 142, or any value or range there between, genes selected from the group of genes listed in Table 5. In some embodiments, the one or more clinical characteristics of the reference data set of step a″, include 1, 2, 3, 4, 5, 6, 7, or 8, clinical characteristics selected from the group of clinical characteristics listed in Table 6. In some embodiments, the plurality of genes of the reference data set of step a″, include at least 2 genes selected from a group of genes listed in any one or more of Tables 1, 2, 3, 4, 5 and 9, and the one or more clinical characteristics of the reference data set of step a″, include 1, 2, 3, 4, 5, 6, 7, or 8 clinical characteristics selected from the group of clinical characteristics listed in Table 6. In some embodiments, genes having collinear expression with correlation coefficients (e.g. in non-limiting aspects >0.7 to >0.9) were removed from the reference data set. Collinear gene expression can be measured by any suitable technique, e.g. Pearson correlation coefficient. While determining feature importance values for the plurality of genes has been described, this is merely a non-limiting illustrative example of a techniques that can be practiced. In various embodiments, one or more feature selection techniques are used to determine the gene set that can classify a lung nodule benign or malignant. Feature selection techniques can include least absolute shrinkage and selection operator (Lasso) regression, support vector machine (SVM), regularized trees, decision trees, memetic algorithm, random multinomial logit (RMNL), auto-encoding networks, submodular feature selection, recursive feature elimination, or any combination thereof. In some of these cases, feature importance values need not be calculated for each of the genes in Table 9. The reference biological sample can be a blood sample, isolated peripheral blood mononuclear cells (PBMCs), lung biopsy sample, nasal fluid sample, saliva sample, or any derivative thereof. In some embodiments, the reference biological sample is a blood sample or any derivative thereof. In some embodiments, the reference biological sample is isolated peripheral blood mononuclear cells (PBMCs) or any derivative thereof. In some embodiments, the reference biological sample is a lung biopsy sample, or any derivative thereof. In some embodiments, the reference biological sample is a nasal fluid sample, or any derivative thereof. In some embodiments, the reference biological sample is a saliva sample, or any derivative thereof.

The machine learning model, e.g. of step b″, can be trained using a supervised machine learning algorithm or an unsupervised machine learning algorithm. In some embodiments, the machine learning model, e.g. of step b″, is trained using linear regression, logistic regression, Ridge regression, Lasso regression, elastic net (EN) regression, support vector machine (SVM), gradient boosted machine (GBM), k nearest neighbors (kNN), generalized linear model (GLM), naïve Bayes (NB), neural network, Random Forest (RF), deep learning algorithm, linear discriminant analysis (LDA), a decision tree learning (DTREE), adaptive boosting (ADB), or any combination thereof. In some embodiments, the machine learning model is trained using logistic regression. In some embodiments, the machine learning model is trained using Ridge regression. In some embodiments, the machine learning model is trained using Lasso regression. In some embodiments, the machine learning model is trained using GLM. In some embodiments, the machine learning model is trained using kNN. In some embodiments, the machine learning model is trained using SVM. In some embodiments, the machine learning model is trained using GBM. In some embodiments, the machine learning model is trained using RF. In some embodiments, the machine learning model is trained using NB. In some embodiments, the machine learning model is trained using the EN regression. In some embodiments, the machine learning model is trained using neural network. In some embodiments, the machine learning model is trained using deep learning algorithm. In some embodiments, the machine learning model is trained using LDA. In some embodiments, the machine learning model is trained using DTREE. In some embodiments, the machine learning model is trained using ADB.

The gene set can classify a lung nodule as the malignant lung nodule or the benign lung nodule with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The gene set can classify a lung nodule as the malignant lung nodule or the benign lung nodule with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The gene set can classify a lung nodule as the malignant lung nodule or the benign lung nodule with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The gene set can classify a lung nodule as the malignant lung nodule or the benign lung nodule with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The gene set can classify a lung nodule as the malignant lung nodule or the benign lung nodule with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The gene set can classify a lung nodule as the malignant lung nodule or the benign lung nodule with an accuracy of about 80% to about 100%. The gene set can classify a lung nodule as the malignant lung nodule or the benign lung nodule with an accuracy of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The gene set can classify a lung nodule as the malignant lung nodule or the benign lung nodule with an accuracy of about 80%, about 85%, about 90%, about 92%, about 94%, about 95%, about 96%, about 97%, about 98%, about 99%, about 99.5%, or about 100%. The gene set can classify a lung nodule as the malignant lung nodule or the benign lung nodule with an accuracy of at least about 80%, about 85%, about 90%, about 92%, about 94%, about 95%, about 96%, about 97%, about 98%, about 99%, or about 99.5%. The gene set can classify a lung nodule as the malignant lung nodule or the benign lung nodule with an accuracy of at most about 85%, about 90%, about 92%, about 94%, about 95%, about 96%, about 97%, about 98%, about 99%, about 99.5%, or about 100%. The gene set can classify a lung nodule as the malignant lung nodule or the benign lung nodule with a sensitivity of about 80% to about 100%. The gene set can classify a lung nodule as the malignant lung nodule or the benign lung nodule with a sensitivity of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The gene set can classify a lung nodule as the malignant lung nodule or the benign lung nodule with a sensitivity of about 80%, about 85%, about 90%, about 92%, about 94%, about 95%, about 96%, about 97%, about 98%, about 99%, about 99.5%, or about 100%. The gene set can classify a lung nodule as the malignant lung nodule or the benign lung nodule with a sensitivity of at least about 80%, about 85%, about 90%, about 92%, about 94%, about 95%, about 96%, about 97%, about 98%, about 99%, or about 99.5%. The gene set can classify a lung nodule as the malignant lung nodule or the benign lung nodule with a sensitivity of at most about 85%, about 90%, about 92%, about 94%, about 95%, about 96%, about 97%, about 98%, about 99%, about 99.5%, or about 100%. The gene set can classify a lung nodule as the malignant lung nodule or the benign lung nodule with a specificity of about 80% to about 100%. The gene set can classify a lung nodule as the malignant lung nodule or the benign lung nodule with a specificity of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The gene set can classify a lung nodule as the malignant lung nodule or the benign lung nodule with a specificity of about 80%, about 85%, about 90%, about 92%, about 94%, about 95%, about 96%, about 97%, about 98%, about 99%, about 99.5%, or about 100%. The gene set can classify a lung nodule as the malignant lung nodule or the benign lung nodule with a specificity of at least about 80%, about 85%, about 90%, about 92%, about 94%, about 95%, about 96%, about 97%, about 98%, about 99%, or about 99.5%. The gene set can classify a lung nodule as the malignant lung nodule or the benign lung nodule with a specificity of at most about 85%, about 90%, about 92%, about 94%, about 95%, about 96%, about 97%, about 98%, about 99%, about 99.5%, or about 100%. The gene set can classify a lung nodule as the malignant lung nodule or the benign lung nodule with a positive predictive value of about 80% to about 100%. The gene set can classify a lung nodule as the malignant lung nodule or the benign lung nodule with a positive predictive value of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The gene set can classify a lung nodule as the malignant lung nodule or the benign lung nodule with a positive predictive value of about 80%, about 85%, about 90%, about 92%, about 94%, about 95%, about 96%, about 97%, about 98%, about 99%, about 99.5%, or about 100%. The gene set can classify a lung nodule as the malignant lung nodule or the benign lung nodule with a positive predictive value of at least about 80%, about 85%, about 90%, about 92%, about 94%, about 95%, about 96%, about 97%, about 98%, about 99%, or about 99.5%. The gene set can classify a lung nodule as the malignant lung nodule or the benign lung nodule with a positive predictive value of at most about 85%, about 90%, about 92%, about 94%, about 95%, about 96%, about 97%, about 98%, about 99%, about 99.5%, or about 100%. The gene set can classify a lung nodule as the malignant lung nodule or the benign lung nodule with a negative predictive value of about 80% to about 100%. The gene set can classify a lung nodule as the malignant lung nodule or the benign lung nodule with a negative predictive value of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The gene set can classify a lung nodule as the malignant lung nodule or the benign lung nodule with a negative predictive value of about 80%, about 85%, about 90%, about 92%, about 94%, about 95%, about 96%, about 97%, about 98%, about 99%, about 99.5%, or about 100%. The gene set can classify a lung nodule as the malignant lung nodule or the benign lung nodule with a negative predictive value of at least about 80%, about 85%, about 90%, about 92%, about 94%, about 95%, about 96%, about 97%, about 98%, about 99%, or about 99.5%. The gene set can classify a lung nodule as the malignant lung nodule or the benign lung nodule with a negative predictive value of at most about 85%, about 90%, about 92%, about 94%, about 95%, about 96%, about 97%, about 98%, about 99%, about 99.5%, or about 100%.

In another aspect, the present disclosure provides a method for developing a trained machine learning model capable of inferring whether a lung nodule of a patient is benign or malignant. The method can include any one of, any combination of, or all of steps a′″, b″′, c′″, d′″ and e′″. Step a′″, can include obtaining and/or providing a first reference data set. The first reference data set can contain a plurality of first individual reference data sets. A respective first individual reference data set of the plurality of first individual reference data sets can contain i) gene expression measurements of a plurality of genes of a reference biological sample from a reference subject having a lung nodule, ii) data regarding whether the lung nodule of the reference subject is benign or malignant, and iii) optionally clinical characteristics data of one or more clinical characteristics selected from the group of clinical characteristics listed in Table 6 of the reference subject. The plurality of first individual reference data sets can be obtained from a plurality of reference subjects. In some embodiments, different first individual reference data sets are obtained from different reference subjects. In some embodiments, each of the first individual reference data set contains i) gene expression measurements of the plurality of genes of a reference biological sample from one reference subject, iii) data regarding whether the lung nodule of the one reference subject is benign or malignant and iii) optionally clinical characteristics data of the one or more clinical characteristics selected from the group of clinical characteristics listed in Table 6 of the one reference subject, wherein different first individual reference data sets are obtained from different reference subjects. A first portion of the plurality of reference subjects can have benign lung nodule, and a second portion of the plurality of reference subjects can have malignant lung nodule. The first reference data set can contain gene expression measurements of the plurality of genes of a plurality of reference biological samples from the plurality of reference subjects having lung nodule; data regarding whether the lung nodules of the reference subjects are benign or malignant; and optionally clinical characteristics data of the reference subjects of one or more clinical characteristics selected from the group of clinical characteristics listed in Table 6. In step b″′, a first machine learning model can be trained using the first reference data set to infer whether a lung nodule is benign or malignant, based at least in part on one or more predictors selected from i) the plurality of genes, and ii) optionally the one or more clinical characteristics. The first machine learning model can be trained to infer whether the lung nodule from a subject is benign or malignant, based at least in part on i) the gene expression measurement data of the plurality of genes of a biological sample from the subject, and ii) optionally the clinical characteristics data of the one or more clinical characteristics of the subject. In some embodiments, the first machine learning model is trained using a training data set containing a first portion of the first reference data set, and a validation data set containing a second portion of the first reference data set. In step c′″, feature importance values of one or more predictors of the first machine learning model can be determined. In step d′″, A predictors of the first machine learning model based at least in part on the feature importance values can be selected, where A can be an integer from 3 to 2000, such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 300, 400, 500, 600, 700, 800, 900, 1000, 1100, 1200, 1300, 1400, 1500, 1600, 1700, 1800, 1900, or 2000, or any integer value or ranges therein. In certain embodiments, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, or 250, or any value or range there between, predictors of the first machine learning model are selected. In some embodiments, the A predictors have top A feature importance values, for example, in a non-limiting aspect, A is 10, and 10 predictors having 10 highest feature importance values are selected. In some embodiments, the feature importance of the A predictors, have an accuracy, greater than 30%, 35%, 40%, 45%, 50%, 55%, 60%, 65%, 70%, 75%, 80% or 90%. In some embodiments, the feature importance of the A predictors, can have a threshold importance, greater than 30, 35, 40, 45, 50, 55, 60, 65, 70, 75, 80 or 90. A predictors can include one or more genes, and/or optionally one or more clinical characteristics. While determining feature importance values for the one or more predictors has been described, this is merely a non-limiting illustrative example of a technique that can be practiced. In various embodiments, one or more feature selection techniques are used to determine the A predictors. Feature selection techniques can include least absolute shrinkage and selection operator (Lasso) regression, support vector machine (SVM), regularized trees, decision trees, memetic algorithm, random multinomial logit (RMNL), auto-encoding networks, submodular feature selection, recursive feature elimination, or any combination thereof. In some of these cases, in step c″′, feature importance values need not be calculated for each of the predictors of first machine learning model. Step e′″, can include training a second machine learning model based at least in part on a second reference data set to obtain the trained machine learning model. The trained machine learning model can infer whether a lung nodule of a subject is benign or malignant, based at least in part on measurement data of the A predictors of the subject. The second reference data set can contain a plurality of second individual reference data sets. A respective second individual reference data set of the plurality of second individual reference data sets can include i) measurement data of the A predictors of a reference subject, and ii) data regarding whether the lung nodule of the reference subject is benign or malignant. Measurement data of the A predictors can include, gene expression measurements of the reference biological sample of the one or more genes predictors of the A predictors, and/or optionally clinical characteristics data of optional one or more clinical characteristics predictors of the A predictors. The plurality of second individual reference data sets can be obtained from the plurality of reference subjects. In some embodiments, different second individual reference data sets are obtained from different reference subjects. In some embodiments, each of the second individual reference data set contains i) measurement data of the A predictors of one reference subject, and ii) data regarding whether the lung nodule of the one reference subject is benign or malignant, wherein different second individual reference data sets are obtained from different reference subjects. In certain embodiments, oversampling or undersampling correction are made during training of the first and/or second machine learning model. The second reference data set can contain measurement data of the A predictors from the plurality of reference subjects, and data regarding whether the lung nodules of the reference subjects are benign or malignant. In some embodiments, the plurality of genes of the first reference data set include at least 2 genes selected from the group of genes listed in Table 9. In some embodiments, the plurality of genes of the first reference data set include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 300, 400, 500, 600, 700, 800, 900, 1000, 1100 or 1178, or any value or range there between, genes selected from the group of genes listed in Table 9. In some embodiments, the plurality of genes of the first reference data set include at least 2 genes selected from the group of genes listed in Table 1. In some embodiments, the plurality of genes of the first reference data set include at least 2 genes selected from the group of genes listed in Table 2. In some embodiments, the plurality of genes of the first reference data set include at least 2 genes selected from the group of genes listed in Table 3. In some embodiments, the plurality of genes of the first reference data set include at least 2 genes selected from the group of genes listed in Table 4. In some embodiments, the plurality of genes of the first reference data set include at least 2 genes selected from the group of genes listed in Table 5. In some embodiments, the one or more clinical characteristics of the first reference data set include 1, 2, 3, 4, 5, 6, 7, or 8 clinical characteristics selected from the group of clinical characteristics listed in Table 6. In some embodiments, the plurality of genes of the first reference data set include at least 2 genes selected from a group of genes listed in any one or more of Tables 1, 2, 3, 4, 5 or 9, and the one or more clinical characteristics of the first reference data set include 1, 2, 3, 4, 5, 6, 7, or 8 clinical characteristics selected from the group of clinical characteristics listed in Table 6. In some embodiments, genes having collinear expression with correlation coefficients (e.g. in non-limiting aspects >0.7 to >0.9) were removed from the reference data set. Collinear gene expression can be measured by any suitable technique, e.g. Pearson correlation coefficient. In some embodiments, the A predictors include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, or 182, or any value or range there between, genes selected from the group of genes listed in Table 1, as predictors. In some embodiments, the A predictors include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, or 175, or any value or range there between, genes selected from the group of genes listed in Table 2, as predictors. In some embodiments, the A predictors include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 or 62, or any value or range there between, genes selected from the group of genes listed in Table 3, as predictors. In some embodiments, the A predictors include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295, or any value or range there between, genes selected from the group of genes listed in Table 4, as predictors. In some embodiments, the A predictors include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, or 142, or any value or range there between, genes selected from the group of genes listed in Table 5, as predictors. In some embodiments, the A predictors include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes of genes selected from the group listed in Table 7, as predictors. In some embodiments, the A predictors can include at least 2, 3, 4, 5, 6, 7, 8, 9, 10, 11, 12, 13, 14, 15, 16, 17, 18, 19, 20, or 21 genes selected from the group of genes listed in Table 8, as predictors. In some embodiments, the A predictors include i) at least, 2 genes selected from the group of genes listed in any one or more of Tables 1, 2, 3, 4, 5, 7, and 8, and ii) optionally size of the nodule, age of the patient, presence of the nodule in the lung upper lobe, or any combination thereof, as predictors. In some embodiments, the A predictors can include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 predictors selected from the group listed in Table 7. In some embodiments, the A predictors comprise the 34 predictors listed in Table 7. In some embodiments, the A predictors consist the 34 predictors listed in Table 7.

In some embodiments, the reference biological sample is a blood sample, isolated peripheral blood mononuclear cells (PBMCs), a lung biopsy sample, nasal fluid, saliva, or any derivative thereof. In some embodiments, the reference biological sample is a blood sample or any derivative thereof. In some embodiments, the reference biological sample is isolated peripheral blood mononuclear cells (PBMCs) or any derivative thereof. In some embodiments, the reference biological sample is a lung biopsy sample, or any derivative thereof. In some embodiments, the reference biological sample is a nasal fluid sample, or any derivative thereof. In some embodiments, the reference biological sample is a saliva sample, or any derivative thereof. The trained machine learning model, e.g. obtained in step e′″, can infer whether a lung nodule is benign or malignant with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The trained machine learning model, e.g. obtained in step e′″, can infer whether a lung nodule is benign or malignant with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The trained machine learning model, e.g. obtained in step e′″, can infer whether a lung nodule is benign or malignant with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The trained machine learning model, e.g. obtained in step e′″, can infer whether a lung nodule is benign or malignant with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The trained machine learning model, e.g. obtained in step e′″, can infer whether a lung nodule is benign or malignant with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The trained machine learning model, e.g. obtained in step e′″, can infer whether a lung nodule is benign or malignant with a receiver operating characteristic (ROC) curve with an Area-Under-Curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99. The trained machine learning model, e.g. obtained in step e′″, can infer whether a lung nodule is benign or malignant with an accuracy of about 80% to about 100%. The trained machine learning model, e.g. obtained in step e′″, can infer whether a lung nodule is benign or malignant with an accuracy of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The trained machine learning model, e.g. obtained in step e′″, can infer whether a lung nodule is benign or malignant with an accuracy of about 80%, about 85%, about 90%, about 92%, about 94%, about 95%, about 96%, about 97%, about 98%, about 99%, about 99.5%, or about 100%. The trained machine learning model, e.g. obtained in step e′″, can infer whether a lung nodule is benign or malignant with an accuracy of at least about 80%, about 85%, about 90%, about 92%, about 94%, about 95%, about 96%, about 97%, about 98%, about 99%, or about 99.5%. The trained machine learning model, e.g. obtained in step e′″, can infer whether a lung nodule is benign or malignant with an accuracy of at most about 85%, about 90%, about 92%, about 94%, about 95%, about 96%, about 97%, about 98%, about 99%, about 99.5%, or about 100%. The trained machine learning model, e.g. obtained in step e′″, can infer whether a lung nodule is benign or malignant with a sensitivity of about 80% to about 100%. The trained machine learning model, e.g. obtained in step e′″, can infer whether a lung nodule is benign or malignant with a sensitivity of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The trained machine learning model, e.g. obtained in step e′″, can infer whether a lung nodule is benign or malignant with a sensitivity of about 80%, about 85%, about 90%, about 92%, about 94%, about 95%, about 96%, about 97%, about 98%, about 99%, about 99.5%, or about 100%. The trained machine learning model, e.g. obtained in step e′″, can infer whether a lung nodule is benign or malignant with a sensitivity of at least about 80%, about 85%, about 90%, about 92%, about 94%, about 95%, about 96%, about 97%, about 98%, about 99%, or about 99.5%. The trained machine learning model, e.g. obtained in step e′″, can infer whether a lung nodule is benign or malignant with a sensitivity of at most about 85%, about 90%, about 92%, about 94%, about 95%, about 96%, about 97%, about 98%, about 99%, about 99.5%, or about 100%. The trained machine learning model, e.g. obtained in step e′″, can infer whether a lung nodule is benign or malignant with a positive predictive value of about 80% to about 100%. The trained machine learning model, e.g. obtained in step e′″, can infer whether a lung nodule is benign or malignant with a positive predictive value of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The trained machine learning model, e.g. obtained in step e′″, can infer whether a lung nodule is benign or malignant with a positive predictive value of about 80%, about 85%, about 90%, about 92%, about 94%, about 95%, about 96%, about 97%, about 98%, about 99%, about 99.5%, or about 100%. The trained machine learning model, e.g. obtained in step e′″, can infer whether a lung nodule is benign or malignant with a positive predictive value of at least about 80%, about 85%, about 90%, about 92%, about 94%, about 95%, about 96%, about 97%, about 98%, about 99%, or about 99.5%. The trained machine learning model, e.g. obtained in step e′″, can infer whether a lung nodule is benign or malignant with a positive predictive value of at most about 85%, about 90%, about 92%, about 94%, about 95%, about 96%, about 97%, about 98%, about 99%, about 99.5%, or about 100%. The trained machine learning model, e.g. obtained in step e′″, can infer whether a lung nodule is benign or malignant with a negative predictive value of about 80% to about 100%. The trained machine learning model, e.g. obtained in step e′″, can infer whether a lung nodule is benign or malignant with a negative predictive value of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The trained machine learning model, e.g. obtained in step e′″, can infer whether a lung nodule is benign or malignant with a negative predictive value of about 80%, about 85%, about 90%, about 92%, about 94%, about 95%, about 96%, about 97%, about 98%, about 99%, about 99.5%, or about 100%. The trained machine learning model, e.g. obtained in step e′″, can infer whether a lung nodule is benign or malignant with a negative predictive value of at least about 80%, about 85%, about 90%, about 92%, about 94%, about 95%, about 96%, about 97%, about 98%, about 99%, or about 99.5%. The trained machine learning model, e.g. obtained in step e′″, can infer whether a lung nodule is benign or malignant with a negative predictive value of at most about 85%, about 90%, about 92%, about 94%, about 95%, about 96%, about 97%, about 98%, about 99%, about 99.5%, or about 100%. The trained machine learning model, e.g. obtained in step e′″, can infer whether a lung nodule is benign or malignant with a ROC curve with an AUC of about 0.8 to about 1. The trained machine learning model, e.g. obtained in step e′″, can infer whether a lung nodule is benign or malignant with a ROC curve with an AUC of about 0.8 to about 0.85, about 0.8 to about 0.9, about 0.8 to about 0.92, about 0.8 to about 0.94, about 0.8 to about 0.95, about 0.8 to about 0.96, about 0.8 to about 0.97, about 0.8 to about 0.98, about 0.8 to about 0.99, about 0.8 to about 0.995, about 0.8 to about 1, about 0.85 to about 0.9, about 0.85 to about 0.92, about 0.85 to about 0.94, about 0.85 to about 0.95, about 0.85 to about 0.96, about 0.85 to about 0.97, about 0.85 to about 0.98, about 0.85 to about 0.99, about 0.85 to about 0.995, about 0.85 to about 1, about 0.9 to about 0.92, about 0.9 to about 0.94, about 0.9 to about 0.95, about 0.9 to about 0.96, about 0.9 to about 0.97, about 0.9 to about 0.98, about 0.9 to about 0.99, about 0.9 to about 0.995, about 0.9 to about 1, about 0.92 to about 0.94, about 0.92 to about 0.95, about 0.92 to about 0.96, about 0.92 to about 0.97, about 0.92 to about 0.98, about 0.92 to about 0.99, about 0.92 to about 0.995, about 0.92 to about 1, about 0.94 to about 0.95, about 0.94 to about 0.96, about 0.94 to about 0.97, about 0.94 to about 0.98, about 0.94 to about 0.99, about 0.94 to about 0.995, about 0.94 to about 1, about 0.95 to about 0.96, about 0.95 to about 0.97, about 0.95 to about 0.98, about 0.95 to about 0.99, about 0.95 to about 0.995, about 0.95 to about 1, about 0.96 to about 0.97, about 0.96 to about 0.98, about 0.96 to about 0.99, about 0.96 to about 0.995, about 0.96 to about 1, about 0.97 to about 0.98, about 0.97 to about 0.99, about 0.97 to about 0.995, about 0.97 to about 1, about 0.98 to about 0.99, about 0.98 to about 0.995, about 0.98 to about 1, about 0.99 to about 0.995, about 0.99 to about 1, or about 0.995 to about 1. The trained machine learning model, e.g. obtained in step e′″, can infer whether a lung nodule is benign or malignant with a ROC curve with an AUC of about 0.8, about 0.85, about 0.9, about 0.92, about 0.94, about 0.95, about 0.96, about 0.97, about 0.98, about 0.99, about 0.995, or about 1. The trained machine learning model, e.g. obtained in step e′″, can infer whether a lung nodule is benign or malignant with a ROC curve with an AUC of at least about 0.8, about 0.85, about 0.9, about 0.92, about 0.94, about 0.95, about 0.96, about 0.97, about 0.98, about 0.99, or about 0.995. The trained machine learning model, e.g. obtained in step e′″, can infer whether a lung nodule is benign or malignant with a ROC curve with an AUC of at most about 0.85, about 0.9, about 0.92, about 0.94, about 0.95, about 0.96, about 0.97, about 0.98, about 0.99, about 0.995, or about 1.

Gene expression data can be obtained by a data analysis tool selected from the group: a BIG-C™ big data analysis tool, an I-Scope™ big data analysis tool, a T-Scope™ big data analysis tool, a Cell Scan big data analysis tool, an MS (Molecular Signature) Scoring™ analysis tool, and a Gene Set Variation Analysis (GSVA) tool (e.g., P-Scope).

In some embodiments, the trained machine learning model is a supervised machine learning algorithm or an unsupervised machine learning algorithm. In some embodiments, first and/or second machine-learning model is independently trained using linear regression, logistic regression (LOG), Ridge regression, Lasso regression, elastic net (EN) regression, support vector machine (SVM), gradient boosted machine (GBM), k nearest neighbors (kNN), generalized linear model (GLM), naïve Bayes (NB), neural network, Random Forest (RF), deep learning algorithm, linear discriminant analysis (LDA), a decision tree learning (DTREE), adaptive boosting (ADB), or any combination thereof. In some embodiments, the first and/or second machine-learning model is independently trained using LOG. In some embodiments, the first and/or second machine-learning model is independently trained using Ridge regression. In some embodiments, the first and/or second machine-learning model is independently trained using Lasso regression. In some embodiments, the first and/or second machine-learning model is independently trained using GLM. In some embodiments, the first and/or second machine-learning model is independently trained using kNN. In some embodiments, the first and/or second machine-learning model is independently trained using SVM. In some embodiments, the first and/or second machine-learning model is independently trained using GBM. In some embodiments, the first and/or second machine-learning model is independently trained using RF. In some embodiments, the first and/or second machine-learning model is independently trained using NB. In some embodiments, the first and/or second machine-learning model is independently trained using the EN regression. In some embodiments, the first and/or second machine-learning model is independently trained using neural network. In some embodiments, the first and/or second machine-learning model is independently trained using deep learning algorithm. In some embodiments, the first and/or second machine-learning model is independently trained using LDA. In some embodiments, the first and/or second machine-learning model is independently trained using DTREE. In some embodiments, the first and/or second machine-learning model is independently trained using ADB.

In an aspect, the present disclosure provides a method for treating lung cancer in a patient. In some embodiments, the patient has a lung nodule. The method can include, any one of, any combination of, or all of steps a″″, b″″, c″″ and d″″. Step a″″, can include obtaining a data set containing i) gene expression measurements of a biological sample from the patient, of at least two lung disease-associated genes, and ii) optionally clinical characteristics data of one or more clinical characteristics selected from the group of clinical characteristics listed in Table 6 of the patient. The gene expression measurements can be obtained by assaying the biological sample. Step b″″, can include providing the data set as an input to a machine-learning model trained to generate an inference of whether the data set is indicative of the patient having or not having lung cancer. In some embodiments, the inference infer whether the data set is indicative of the lung nodule of the patient is malignant or benign. Step c″″, can include receiving, as an output of the machine-learning model, the inference indicating whether the data set is indicative of the patient having or not having lung cancer. In some embodiments, the inference received as an output, indicate whether the lung nodule of the patient is malignant lung nodule or the benign lung nodule. Step d″″, can include administering a treatment based on the determination that the patient has lung cancer. In some embodiments, the treatment is administering based on the patient's lung nodule being classified as a malignant nodule.

The data set of step a″″, can contain i) gene expression measurements of the biological sample from the patient, of at least two lung disease-associated genes selected from the group of genes listed in any one or more of Tables 1, 2, 3, 4, 5, 7, and 8, and ii) optionally clinical characteristics data of one or more clinical characteristics selected from the group of clinical characteristics listed in Table 6 of the patient. In some embodiments, the at least two lung disease-associated genes of the data set of step a″″, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, or 182, or any value or range there between, genes selected from the group of genes listed in Table 1. In some embodiments, the at least two lung disease-associated genes of the data set of step a″″, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, or 175, or any value or range there between, genes selected from the group of genes listed in Table 2. In some embodiments, the at least two lung disease-associated genes of the data set of step a″″, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 or 62, or any value or range there between, genes selected from the group of genes listed in Table 3. In some embodiments, the at least two lung disease-associated genes of the data set of step a″″, include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295, or any value or range there between, genes selected from the group of genes listed in Table 4. In some embodiments, the at least two lung disease-associated genes of the data set of step a″″, include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, or 142, or any value or range there between, genes selected from the group of genes listed in Table 5. In some embodiments, the at least two lung disease-associated genes of the data set of step a″″, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7. In some embodiments, the at least two lung disease-associated genes of the data set of step a″″, are selected from BCAT1, CRCP, COA4, OVCA2, POM121, HLA-DPA1, VPS37C, MGST2, RNF220, HDAC3, NFE2L1, WDR20, CNPY4, HOXB2, C6orf120, TMEM8A, ASAP1-IT2, C15orf54, CD101, FNBP1, TECR, PROK2, SLC35B3, TDRD9, CLHC1, LPL, IFITM3, OGFOD3, EIF2B3, TMEM65, and MKRN3. In some embodiments, the at least two lung disease-associated genes of the data set of step a″″, include at least 2, 3, 4, 5, 6, 7, 8, 9, 10, 11, 12, 13, 14, 15, 16, 17, 18, 19, 20, or 21 genes selected from the group of genes listed in Table 8. In some embodiments, the at least two lung disease-associated genes of step a″″, are selected from BCAT1, USP32P2, CD177, QPCT, SCAF4, SNRPD3, BCL9L, THBS1, SLC22A18AS, ARCN1, DHX16, SATB1, ST6GAL1, CXCL1, TDRD9, ZNF831, MTCH1, FAM86HP, DHX8, RNF114, and DCTN4. In some embodiments, the one or more clinical characteristics of the data set of step a″″, include 1, 2, 3, 4, 5, 6, 7 or 8 clinical characteristics selected from the group of clinical characteristics listed in Table 6. In some embodiments, the one or more clinical characteristics of the data set of step a″″, include size of the nodule, age of the patient, presence of the nodule in the lung upper lobe, or any combination thereof. In some embodiments, the data set of step a″″, contains i) gene expression measurements of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7, and ii) clinical characteristics data of 1, 2, 3, 4, 5, 6, 7 or 8 clinical characteristics selected from the group of clinical characteristics listed in Table 6, of the patient. In some embodiments, the data set of step a″″, contains i) gene expression measurements of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7, and ii) clinical characteristics data of the clinical characteristics of the patient selected fromsize of the nodule, age of the patient, presence of the nodule in the lung upper lobe, or any combination thereof. In some embodiments, the at least two lung disease-associated genes of step a″″, comprise the 31 genes listed in Table 7, and the one or more clinical characteristics of step a″″ comprise the size of the nodule, age of the patient, and the presence of the nodule in the lung upper lobe. In some embodiments, the at least two lung disease-associated genes of step a″″, consist of the 31 genes listed in Table 7, and the one or more clinical characteristics of step a″″ consist of the size of the nodule, age of the patient, and the presence of the nodule in the lung upper lobe.

The inference from the machine learning model can include a confidence value between 0 and 1, such as, 0, 0.1, 0.2, 0.3, 0.4, 0.5, 0.6, 0.7, 0.8, 0.9 or 1, or any value or range there between, that the nodule is malignant, where higher confidence values may be correlated with a higher likelihood that the nodule is malignant. In some embodiments, the biological sample is a blood sample, isolated peripheral blood mononuclear cells (PBMCs), a lung biopsy sample, nasal fluid, saliva, or any derivative thereof. In some embodiments, the biological sample is a blood sample or any derivative thereof. In some embodiments, the biological sample is isolated peripheral blood mononuclear cells (PBMCs) or any derivative thereof. In some embodiments, the biological sample is a lung biopsy sample or any derivative thereof. In some embodiments, the biological sample is a nasal fluid sample or any derivative thereof. In some embodiments, the biological sample is a saliva sample or any derivative thereof. In certain embodiments, the method includes optionally performing biopsy of the lung nodule of the patient based at least in part on the classification of the lung nodule of the patient as the malignant lung nodule or the benign lung nodule. In certain embodiments, the method includes optionally performing a biopsy of the lung nodule of the patient based at least in part on the classification of the lung nodule of the patient as the malignant lung nodule. The decision to perform biopsy may depend on confidence value of the inference. The machine-learning model, e.g. of step b″″, can generate the inference of whether the data set is indicative of the patient having a malignant lung nodule or a benign lung nodule, wherein the patient having malignant lung nodule may indicate the patient has lung cancer, and the patient having benign lung nodule may indicate the patient does not have lung cancer. In certain embodiments, biopsy of the lung nodule of the patient is not performed. The machine-learning model of step b″″, can be trained according to a method described herein, e.g. according to the methods training of the machine-learning model of step b′.

The machine learning model of step b″″, can infer whether the data set is indicative of the patient having or not having lung cancer with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In some embodiments, the machine learning model of step b″″, can infer whether the data set is indicative of the patient having the malignant lung nodule or benign lung nodule with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model of step b″″, can infer whether the data set is indicative of the patient having or not having lung cancer with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In some embodiments, the machine learning model of step b″″, can infer whether the data set is indicative of the patient having the malignant lung nodule or benign lung nodule with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model of step b″″, can infer whether the data set is indicative of the patient having or not having lung cancer with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In some embodiments, the machine learning model of step b″″, can infer whether the data set is indicative of the patient having the malignant lung nodule or benign lung nodule with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model of step b″″, can infer whether the data set is indicative of the patient having or not having lung cancer with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In some embodiments, the machine learning model of step b″″, can infer whether the data set is indicative of the patient having the malignant lung nodule or benign lung nodule with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model of step b″″, can infer whether the data set is indicative of the patient having or not having lung cancer with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. In some embodiments, the machine learning model of step b″″, can infer whether the data set is indicative of the patient having the malignant lung nodule or benign lung nodule with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model of step b″″, can infer whether the data set is indicative of the patient having or not having lung cancer with a receiver operating characteristic (ROC) curve with an Area-Under-Curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99. In some embodiments, the machine learning model of step b″″, can infer whether the data set is indicative of the patient having the malignant lung nodule or benign lung nodule with a receiver operating characteristic (ROC) curve with an Area-Under-Curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99. The machine learning model of step b″″, can infer whether the data set is indicative of the patient having or not having lung cancer with an accuracy of about 80% to about 100%. The machine learning model of step b″″, can infer whether the data set is indicative of the patient having or not having lung cancer with an accuracy of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The machine learning model of step b″″, can infer whether the data set is indicative of the patient having or not having lung cancer with an accuracy of about 80%, about 85%, about 90%, about 92%, about 94%, about 95%, about 96%, about 97%, about 98%, about 99%, about 99.5%, or about 100%. The machine learning model of step b″″, can infer whether the data set is indicative of the patient having or not having lung cancer with an accuracy of at least about 80%, about 85%, about 90%, about 92%, about 94%, about 95%, about 96%, about 97%, about 98%, about 99%, or about 99.5%. The machine learning model of step b″″, can infer whether the data set is indicative of the patient having or not having lung cancer with an accuracy of at most about 85%, about 90%, about 92%, about 94%, about 95%, about 96%, about 97%, about 98%, about 99%, about 99.5%, or about 100%. The machine learning model of step b″″, can infer whether the data set is indicative of the patient having or not having lung cancer with a sensitivity of about 80% to about 100%. The machine learning model of step b″″, can infer whether the data set is indicative of the patient having or not having lung cancer with a sensitivity of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The machine learning model of step b″″, can infer whether the data set is indicative of the patient having or not having lung cancer with a sensitivity of about 80%, about 85%, about 90%, about 92%, about 94%, about 95%, about 96%, about 97%, about 98%, about 99%, about 99.5%, or about 100%. The machine learning model of step b″″, can infer whether the data set is indicative of the patient having or not having lung cancer with a sensitivity of at least about 80%, about 85%, about 90%, about 92%, about 94%, about 95%, about 96%, about 97%, about 98%, about 99%, or about 99.5%. The machine learning model of step b″″, can infer whether the data set is indicative of the patient having or not having lung cancer with a sensitivity of at most about 85%, about 90%, about 92%, about 94%, about 95%, about 96%, about 97%, about 98%, about 99%, about 99.5%, or about 100%. The machine learning model of step b″″, can infer whether the data set is indicative of the patient having or not having lung cancer with a specificity of about 80% to about 100%. The machine learning model of step b″″, can infer whether the data set is indicative of the patient having or not having lung cancer with a specificity of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The machine learning model of step b″″, can infer whether the data set is indicative of the patient having or not having lung cancer with a specificity of about 80%, about 85%, about 90%, about 92%, about 94%, about 95%, about 96%, about 97%, about 98%, about 99%, about 99.5%, or about 100%. The machine learning model of step b″″, can infer whether the data set is indicative of the patient having or not having lung cancer with a specificity of at least about 80%, about 85%, about 90%, about 92%, about 94%, about 95%, about 96%, about 97%, about 98%, about 99%, or about 99.5%. The machine learning model of step b″″, can infer whether the data set is indicative of the patient having or not having lung cancer with a specificity of at most about 85%, about 90%, about 92%, about 94%, about 95%, about 96%, about 97%, about 98%, about 99%, about 99.5%, or about 100%. The machine learning model of step b″″, can infer whether the data set is indicative of the patient having or not having lung cancer with a positive predictive value of about 80% to about 100%. The machine learning model of step b″″, can infer whether the data set is indicative of the patient having or not having lung cancer with a positive predictive value of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The machine learning model of step b″″, can infer whether the data set is indicative of the patient having or not having lung cancer with a positive predictive value of about 80%, about 85%, about 90%, about 92%, about 94%, about 95%, about 96%, about 97%, about 98%, about 99%, about 99.5%, or about 100%. The machine learning model of step b″″, can infer whether the data set is indicative of the patient having or not having lung cancer with a positive predictive value of at least about 80%, about 85%, about 90%, about 92%, about 94%, about 95%, about 96%, about 97%, about 98%, about 99%, or about 99.5%. The machine learning model of step b″″, can infer whether the data set is indicative of the patient having or not having lung cancer with a positive predictive value of at most about 85%, about 90%, about 92%, about 94%, about 95%, about 96%, about 97%, about 98%, about 99%, about 99.5%, or about 100%. The machine learning model of step b″″, can infer whether the data set is indicative of the patient having or not having lung cancer with a negative predictive value of about 80% to about 100%. The machine learning model of step b″″, can infer whether the data set is indicative of the patient having or not having lung cancer with a negative predictive value of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The machine learning model of step b″″, can infer whether the data set is indicative of the patient having or not having lung cancer with a negative predictive value of about 80%, about 85%, about 90%, about 92%, about 94%, about 95%, about 96%, about 97%, about 98%, about 99%, about 99.5%, or about 100%. The machine learning model of step b″″, can infer whether the data set is indicative of the patient having or not having lung cancer with a negative predictive value of at least about 80%, about 85%, about 90%, about 92%, about 94%, about 95%, about 96%, about 97%, about 98%, about 99%, or about 99.5%. The machine learning model of step b″″, can infer whether the data set is indicative of the patient having or not having lung cancer with a negative predictive value of at most about 85%, about 90%, about 92%, about 94%, about 95%, about 96%, about 97%, about 98%, about 99%, about 99.5%, or about 100%. The machine learning model of step b″″, can infer whether the data set is indicative of the patient having or not having lung cancer with a ROC curve with an AUC of about 0.8 to about 1. The machine learning model of step b″″, can infer whether the data set is indicative of the patient having or not having lung cancer with a ROC curve with an AUC of about 0.8 to about 0.85, about 0.8 to about 0.9, about 0.8 to about 0.92, about 0.8 to about 0.94, about 0.8 to about 0.95, about 0.8 to about 0.96, about 0.8 to about 0.97, about 0.8 to about 0.98, about 0.8 to about 0.99, about 0.8 to about 0.995, about 0.8 to about 1, about 0.85 to about 0.9, about 0.85 to about 0.92, about 0.85 to about 0.94, about 0.85 to about 0.95, about 0.85 to about 0.96, about 0.85 to about 0.97, about 0.85 to about 0.98, about 0.85 to about 0.99, about 0.85 to about 0.995, about 0.85 to about 1, about 0.9 to about 0.92, about 0.9 to about 0.94, about 0.9 to about 0.95, about 0.9 to about 0.96, about 0.9 to about 0.97, about 0.9 to about 0.98, about 0.9 to about 0.99, about 0.9 to about 0.995, about 0.9 to about 1, about 0.92 to about 0.94, about 0.92 to about 0.95, about 0.92 to about 0.96, about 0.92 to about 0.97, about 0.92 to about 0.98, about 0.92 to about 0.99, about 0.92 to about 0.995, about 0.92 to about 1, about 0.94 to about 0.95, about 0.94 to about 0.96, about 0.94 to about 0.97, about 0.94 to about 0.98, about 0.94 to about 0.99, about 0.94 to about 0.995, about 0.94 to about 1, about 0.95 to about 0.96, about 0.95 to about 0.97, about 0.95 to about 0.98, about 0.95 to about 0.99, about 0.95 to about 0.995, about 0.95 to about 1, about 0.96 to about 0.97, about 0.96 to about 0.98, about 0.96 to about 0.99, about 0.96 to about 0.995, about 0.96 to about 1, about 0.97 to about 0.98, about 0.97 to about 0.99, about 0.97 to about 0.995, about 0.97 to about 1, about 0.98 to about 0.99, about 0.98 to about 0.995, about 0.98 to about 1, about 0.99 to about 0.995, about 0.99 to about 1, or about 0.995 to about 1. The machine learning model of step b″″, can infer whether the data set is indicative of the patient having or not having lung cancer with a ROC curve with an AUC of about 0.8, about 0.85, about 0.9, about 0.92, about 0.94, about 0.95, about 0.96, about 0.97, about 0.98, about 0.99, about 0.995, or about 1. The machine learning model of step b″″, can infer whether the data set is indicative of the patient having or not having lung cancer with a ROC curve with an AUC of at least about 0.8, about 0.85, about 0.9, about 0.92, about 0.94, about 0.95, about 0.96, about 0.97, about 0.98, about 0.99, or about 0.995. The machine learning model of step b″″, can infer whether the data set is indicative of the patient having or not having lung cancer with a ROC curve with an AUC of at most about 0.85, about 0.9, about 0.92, about 0.94, about 0.95, about 0.96, about 0.97, about 0.98, about 0.99, about 0.995, or about 1.

In some embodiments, the treatment is configured to treat a lung cancer of the patient. In some embodiments, the treatment is configured to reduce a severity of a lung cancer of the patient. In some embodiments, the treatment is configured to reduce a risk of having a lung cancer of the patient. The treatment can include one or more treatments of lung cancer. In some embodiments, the treatment is surgery, chemotherapy, targeted therapy, immunotherapy, radiotherapy, or any combination thereof.

In an aspect, the present disclosure provides a method for assessing a lung nodule of a patient, for biopsy. The method can include, any one of, any combination of, or all of steps w, x, y and z. Step w, can include obtaining a data set containing i) gene expression measurements of a biological sample from the patient, of at least two lung disease-associated genes, and ii) optionally clinical characteristics data of one or more clinical characteristics selected from the group of clinical characteristics listed in Table 6 of the patient. The gene expression measurements can be obtained by assaying the biological sample. Step x, can include providing the data set as an input to a machine learning model trained to generate an inference of whether the data set is indicative of a malignant lung nodule or a benign lung nodule. Step y, can include receiving, as an output of the machine-learning model, the inference indicating whether the data set is indicative of the malignant lung nodule or the benign lung nodule. Step z, can include performing biopsy of the lung nodule based on the machine learning classification of the lung nodule. In some embodiments step z, can include performing biopsy of the lung nodule based on the lung nodule being classified as a malignant nodule or benign nodule. In some embodiments step z, can include performing biopsy of the lung nodule based on the lung nodule being classified as a malignant nodule. The decision to perform biopsy may depend on confidence value of the inference. In certain embodiments, biopsy of the lung nodule of the patient is not performed. In some embodiments, the data set of step w, contains i) gene expression measurements of the biological sample of at least two lung disease-associated genes selected from the group of genes listed in any one or more of Tables 1, 2, 3, 4, 5, 7, and 8, and ii) optionally clinical characteristics data of one or more clinical characteristics of the patient selected from the group of clinical characteristics listed in Table 6. In some embodiments, the at least two lung disease-associated genes of the data set of step w, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, or 182, or any value or range there between, genes selected from the group of genes listed in Table 1. In some embodiments, the at least two lung disease-associated genes of the data set of step w, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, or 175, or any value or range there between, genes selected from the group of genes listed in Table 2. In some embodiments, the at least two lung disease-associated genes of the data set of step w, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 or 62, or any value or range there between, genes selected from the group of genes listed in Table 3. In some embodiments, the at least two lung disease-associated genes of the data set of step w, include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295, or any value or range there between, genes selected from the group of genes listed in Table 4. In some embodiments, the at least two lung disease-associated genes of the data set of step w, include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, or 142, or any value or range there between, genes selected from the group of genes listed in Table 5. In some embodiments, the at least two lung disease-associated genes of the data set of step w, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7. In some embodiments, the at least two lung disease-associated genes of step w, are selected from BCAT1, CRCP, COA4, OVCA2, POM121, HLA-DPA1, VPS37C, MGST2, RNF220, HDAC3, NFE2L1, WDR20, CNPY4, HOXB2, C6orf120, TMEM8A, ASAP1-IT2, C15orf54, CD101, FNBP1, TECR, PROK2, SLC35B3, TDRD9, CLHC1, LPL, IFITM3, OGFOD3, EIF2B3, TMEM65, and MKRN3. In some embodiments, the at least two lung disease-associated genes of the data set of step w, include at least 2, 3, 4, 5, 6, 7, 8, 9, 10, 11, 12, 13, 14, 15, 16, 17, 18, 19, 20, or 21 genes selected from the group of genes listed in Table 8. In some embodiments, the at least two lung disease-associated genes of step w, are selected from BCAT1, USP32P2, CD177, QPCT, SCAF4, SNRPD3, BCL9L, THBS1, SLC22A18AS, ARCN1, DHX16, SATB1, ST6GAL1, CXCL1, TDRD9, ZNF831, MTCH1, FAM86HP, DHX8, RNF114, and DCTN4. In some embodiments, the one or more clinical characteristics of the data set of step w, includes 1, 2, 3, 4, 5, 6, 7 or 8 clinical characteristics selected from the group listed in Table 6, of the patient. In some embodiments, one or more clinical characteristics of the data set of step w, includes size of the nodule, age of the patient, presence of the nodule in the lung upper lobe, or any combination thereof. In some embodiments, the data set of step w, contains i) gene expression measurements of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease-associated genes selected from the group of genes listed in Table 7, and ii) clinical characteristics data of 1, 2, 3, 4, 5, 6, 7 or 8 clinical characteristics selected from the group of clinical characteristics listed in Table 6. In some embodiments, the data set of step w, contains i) gene expression measurements of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease-associated genes selected from the group of genes listed in Table 7, and ii) clinical characteristics data of clinical characteristics of the patient selected from size of the nodule, age of the patient, presence of the nodule in the lung upper lobe, or any combination thereof. In some embodiments, the at least two lung disease-associated genes of step w, comprise the 31 genes listed in Table 7, and the one or more clinical characteristics of step w, comprise the size of the nodule, age of the patient, and the presence of the nodule in the lung upper lobe. In some embodiments, the at least two lung disease-associated genes of step w, consist of the 31 genes listed in Table 7, and the one or more clinical characteristics of step w, consist of the size of the nodule, age of the patient, and the presence of the nodule in the lung upper lobe.

In some embodiments, the biological sample is a blood sample, isolated peripheral blood mononuclear cells (PBMCs), a lung biopsy sample, nasal fluid, saliva, or any derivative thereof. In some embodiments, the biological sample is a blood sample or any derivative thereof. In some embodiments, the biological sample is isolated peripheral blood mononuclear cells (PBMCs) or any derivative thereof. In some embodiments, the biological sample is a lung biopsy sample, or any derivative thereof. In some embodiments, the biological sample is a nasal fluid sample, or any derivative thereof. In some embodiments, the biological sample is a saliva sample, or any derivative thereof.

The machine-learning model, e.g. of step x, can be trained according to a method described herein, e.g. according to the methods training of the machine-learning model of step b′.

Certain aspects are directed to a method for determining lung cancer in a patient. The method can include, any one of, any combination of, or all of steps w′, x′, y′ and z′. Step w′ can include obtaining a data set containing i) gene expression measurements of a biological sample from the patient, of at least two lung disease-associated genes, and ii) optionally clinical characteristics data of one or more clinical characteristics of the patient, selected from a group of clinical characteristics listed in Table 6. The gene expression measurements can be obtained by assaying the biological sample. Step x′ can include providing the data set as an input to a machine-learning model trained to generate an inference of whether the data set is indicative of the patient having or not having lung cancer. Step y′ can include receiving, as an output of the machine-learning model, the inference indicating whether the data set is indicative of the patient having or not having lung cancer. Step z′ can include electronically outputting a report indicating the patient has, or does not have lung cancer. The gene expression measurement of the biological sample can be performed using any suitable technique, such as any suitable RNA quantification technique, including but not limited to RNA-seq, Ampli-seq, or the like. In some embodiments, the data set of step w′, contains i) gene expression measurements of the biological sample of at least two lung disease-associated genes selected from the group of genes listed in any one or more of Tables 1, 2, 3, 4, 5, 7, and 8, and ii) optionally clinical characteristics data of one or more clinical characteristics of the patient selected from the group of clinical characteristics listed in Table 6.

In some embodiments, the at least two lung disease-associated genes of the data set of step w′, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, or 182, or any value or range there between, genes selected from the group of genes listed in Table 1. In some embodiments, the at least two lung disease-associated genes of the data set of step w′, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, or 175, or any value or range there between, genes selected from the group of genes listed in Table 2. In some embodiments, the at least two lung disease-associated genes of the data set of step w′, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 or 62, or any value or range there between, genes selected from the group of genes listed in Table 3. In some embodiments, the at least two lung disease-associated genes of the data set of step w′, include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295, or any value or range there between, genes selected from the group of genes listed in Table 4. In some embodiments, the at least two lung disease-associated genes of the data set of step w′, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, or 142 genes selected from the group of genes listed in Table 5. In some embodiments, the at least two lung disease-associated genes of the data set of step w′, include includes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7. In some embodiments, the at least two lung disease-associated genes of step w′, are selected from BCAT1, CRCP, COA4, OVCA2, POM121, HLA-DPA1, VPS37C, MGST2, RNF220, HDAC3, NFE2L1, WDR20, CNPY4, HOXB2, C6orf120, TMEM8A, ASAP1-IT2, C15orf54, CD101, FNBP1, TECR, PROK2, SLC35B3, TDRD9, CLHC1, LPL, IFITM3, OGFOD3, EIF2B3, TMEM65, and MKRN3. In some embodiments, the at least two lung disease-associated genes of the data set of step w′, include at least 2, 3, 4, 5, 6, 7, 8, 9, 10, 11, 12, 13, 14, 15, 16, 17, 18, 19, 20, or 21 genes selected from the group of genes listed in Table 8. In some embodiments, the at least two lung disease-associated genes of step w, are selected from BCAT1, USP32P2, CD177, QPCT, SCAF4, SNRPD3, BCL9L, THBS1, SLC22A18AS, ARCN1, DHX16, SATB1, ST6GAL1, CXCL1, TDRD9, ZNF831, MTCH1, FAM86HP, DHX8, RNF114, and DCTN4. In some embodiments, the one or more clinical characteristics of the dataset of step w′, includes, 1, 2, 3, 4, 5, 6, 7 or 8 clinical characteristics selected from the group listed in Table 6. In some embodiments, the one or more clinical characteristics of the dataset of step w′, includes size of the nodule. In some embodiments, the one or more clinical characteristics of the dataset of step w′, includes age of the patient. In some embodiments, the one or more clinical characteristics of the dataset of step w′, includes presence of the nodule in the lung upper lobe. In some embodiments, the one or more clinical characteristics of the dataset of step w′, include size of the nodule, age of the patient, presence of the nodule in the lung upper lobe, or any combination thereof. In some embodiments, the data set of step w′, contains i) gene expression measurements of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease associated genes selected from the group of genes listed in Table 7, and ii) clinical characteristics data of 1, 2, 3, 4, 5, 6, 7 or 8 clinical characteristics of the patient selected from the group of clinical characteristics listed in Table 6. In some embodiments, the data set of step w′, contains i) gene expression measurements of the biological sample of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease-associated genes selected from the group of genes listed in Table 7, and ii) clinical characteristics data of the clinical characteristics of the patient selected from size of the nodule, age of the patient, presence of the nodule in the lung upper lobe, or any combination thereof. In some embodiments, the at least two lung disease-associated genes of step w′, comprise the 31 genes listed in Table 7, and the one or more clinical characteristics of step w′, comprise the size of the nodule, age of the patient, and the presence of the nodule in the lung upper lobe. In some embodiments, the at least two lung disease-associated genes of step w′, consist of the 31 genes listed in Table 7, and the one or more clinical characteristics of step w′, consist of the size of the nodule, age of the patient, and the presence of the nodule in the lung upper lobe.

In some embodiments, the biological sample is selected from the group: a blood sample, isolated peripheral blood mononuclear cells (PBMCs), a lung biopsy sample, nasal fluid, saliva, and any derivative thereof. In some embodiments, the biological sample is a blood sample or any derivative thereof. In some embodiments, the biological sample is isolated peripheral blood mononuclear cells (PBMCs) or any derivative thereof. In some embodiments, the biological sample is a lung biopsy sample, or any derivative thereof. In some embodiments, the biological sample is a nasal fluid sample, or any derivative thereof. In some embodiments, the biological sample is a saliva sample, or any derivative thereof.

The method can determine whether the patient has or does not have lung cancer with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The method can determine whether the patient has or does not have lung cancer with an sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The method can determine whether the patient has or does not have lung cancer with an specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The method can determine whether the patient has or does not have lung cancer with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The method can determine whether the patient has or does not have lung cancer with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model, e.g. of step x′, can infer whether the data set is indicative of the patient having or not having lung cancer with a receiver operating characteristic (ROC) curve with an AUC of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99. The method can determine whether the patient has or does not have lung cancer with an accuracy of about 80% to about 100%. The method can determine whether the patient has or does not have lung cancer with an accuracy of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The method can determine whether the patient has or does not have lung cancer with an accuracy of about 80%, about 85%, about 90%, about 92%, about 94%, about 95%, about 96%, about 97%, about 98%, about 99%, about 99.5%, or about 100%. The method can determine whether the patient has or does not have lung cancer with an accuracy of at least about 80%, about 85%, about 90%, about 92%, about 94%, about 95%, about 96%, about 97%, about 98%, about 99%, or about 99.5%. The method can determine whether the patient has or does not have lung cancer with an accuracy of at most about 85%, about 90%, about 92%, about 94%, about 95%, about 96%, about 97%, about 98%, about 99%, about 99.5%, or about 100%. The method can determine whether the patient has or does not have lung cancer with a sensitivity of about 80% to about 100%. The method can determine whether the patient has or does not have lung cancer with a sensitivity of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The method can determine whether the patient has or does not have lung cancer with a sensitivity of about 80%, about 85%, about 90%, about 92%, about 94%, about 95%, about 96%, about 97%, about 98%, about 99%, about 99.5%, or about 100%. The method can determine whether the patient has or does not have lung cancer with a sensitivity of at least about 80%, about 85%, about 90%, about 92%, about 94%, about 95%, about 96%, about 97%, about 98%, about 99%, or about 99.5%. The method can determine whether the patient has or does not have lung cancer with a sensitivity of at most about 85%, about 90%, about 92%, about 94%, about 95%, about 96%, about 97%, about 98%, about 99%, about 99.5%, or about 100%. The method can determine whether the patient has or does not have lung cancer with a specificity of about 80% to about 100%. The method can determine whether the patient has or does not have lung cancer with a specificity of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The method can determine whether the patient has or does not have lung cancer with a specificity of about 80%, about 85%, about 90%, about 92%, about 94%, about 95%, about 96%, about 97%, about 98%, about 99%, about 99.5%, or about 100%. The method can determine whether the patient has or does not have lung cancer with a specificity of at least about 80%, about 85%, about 90%, about 92%, about 94%, about 95%, about 96%, about 97%, about 98%, about 99%, or about 99.5%. The method can determine whether the patient has or does not have lung cancer with a specificity of at most about 85%, about 90%, about 92%, about 94%, about 95%, about 96%, about 97%, about 98%, about 99%, about 99.5%, or about 100%. The method can determine whether the patient has or does not have lung cancer with a positive predictive value of about 80% to about 100%. The method can determine whether the patient has or does not have lung cancer with a positive predictive value of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The method can determine whether the patient has or does not have lung cancer with a positive predictive value of about 80%, about 85%, about 90%, about 92%, about 94%, about 95%, about 96%, about 97%, about 98%, about 99%, about 99.5%, or about 100%. The method can determine whether the patient has or does not have lung cancer with a positive predictive value of at least about 80%, about 85%, about 90%, about 92%, about 94%, about 95%, about 96%, about 97%, about 98%, about 99%, or about 99.5%. The method can determine whether the patient has or does not have lung cancer with a positive predictive value of at most about 85%, about 90%, about 92%, about 94%, about 95%, about 96%, about 97%, about 98%, about 99%, about 99.5%, or about 100%. The method can determine whether the patient has or does not have lung cancer with a negative predictive value of about 80% to about 100%. The method can determine whether the patient has or does not have lung cancer with a negative predictive value of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The method can determine whether the patient has or does not have lung cancer with a negative predictive value of about 80%, about 85%, about 90%, about 92%, about 94%, about 95%, about 96%, about 97%, about 98%, about 99%, about 99.5%, or about 100%. The method can determine whether the patient has or does not have lung cancer with a negative predictive value of at least about 80%, about 85%, about 90%, about 92%, about 94%, about 95%, about 96%, about 97%, about 98%, about 99%, or about 99.5%. The method can determine whether the patient has or does not have lung cancer with a negative predictive value of at most about 85%, about 90%, about 92%, about 94%, about 95%, about 96%, about 97%, about 98%, about 99%, about 99.5%, or about 100%. The machine learning model of step x′, can infer whether the data set is indicative of the patient having or not having lung cancer with a ROC curve with an AUC of about 0.8 to about 1. The machine learning model of step x′, can infer whether the data set is indicative of the patient having or not having lung cancer with a ROC curve with an AUC of about 0.8 to about 0.85, about 0.8 to about 0.9, about 0.8 to about 0.92, about 0.8 to about 0.94, about 0.8 to about 0.95, about 0.8 to about 0.96, about 0.8 to about 0.97, about 0.8 to about 0.98, about 0.8 to about 0.99, about 0.8 to about 0.995, about 0.8 to about 1, about 0.85 to about 0.9, about 0.85 to about 0.92, about 0.85 to about 0.94, about 0.85 to about 0.95, about 0.85 to about 0.96, about 0.85 to about 0.97, about 0.85 to about 0.98, about 0.85 to about 0.99, about 0.85 to about 0.995, about 0.85 to about 1, about 0.9 to about 0.92, about 0.9 to about 0.94, about 0.9 to about 0.95, about 0.9 to about 0.96, about 0.9 to about 0.97, about 0.9 to about 0.98, about 0.9 to about 0.99, about 0.9 to about 0.995, about 0.9 to about 1, about 0.92 to about 0.94, about 0.92 to about 0.95, about 0.92 to about 0.96, about 0.92 to about 0.97, about 0.92 to about 0.98, about 0.92 to about 0.99, about 0.92 to about 0.995, about 0.92 to about 1, about 0.94 to about 0.95, about 0.94 to about 0.96, about 0.94 to about 0.97, about 0.94 to about 0.98, about 0.94 to about 0.99, about 0.94 to about 0.995, about 0.94 to about 1, about 0.95 to about 0.96, about 0.95 to about 0.97, about 0.95 to about 0.98, about 0.95 to about 0.99, about 0.95 to about 0.995, about 0.95 to about 1, about 0.96 to about 0.97, about 0.96 to about 0.98, about 0.96 to about 0.99, about 0.96 to about 0.995, about 0.96 to about 1, about 0.97 to about 0.98, about 0.97 to about 0.99, about 0.97 to about 0.995, about 0.97 to about 1, about 0.98 to about 0.99, about 0.98 to about 0.995, about 0.98 to about 1, about 0.99 to about 0.995, about 0.99 to about 1, or about 0.995 to about 1. The machine learning model of step x′, can infer whether the data set is indicative of the patient having or not having lung cancer with a ROC curve with an AUC of about 0.8, about 0.85, about 0.9, about 0.92, about 0.94, about 0.95, about 0.96, about 0.97, about 0.98, about 0.99, about 0.995, or about 1. The machine learning model of step x′, can infer whether the data set is indicative of the patient having or not having lung cancer with a ROC curve with an AUC of at least about 0.8, about 0.85, about 0.9, about 0.92, about 0.94, about 0.95, about 0.96, about 0.97, about 0.98, about 0.99, or about 0.995. The machine learning model of step x′, can infer whether the data set is indicative of the patient having or not having lung cancer with a ROC curve with an AUC of at most about 0.85, about 0.9, about 0.92, about 0.94, about 0.95, about 0.96, about 0.97, about 0.98, about 0.99, about 0.995, or about 1.

The inference from the machine learning model can include a confidence value between 0 and 1, such as, 0, 0.1, 0.2, 0.3, 0.4, 0.5, 0.6, 0.7, 0.8, 0.9 or 1, or any value or range there between, that the patient has lung cancer. Higher confidence values may be correlated with a higher likelihood that the patient has is lung cancer.

The machine-learning model, e.g. of step x′, can generate inference of whether the data set is indicative of the patient having a malignant lung nodule or a benign lung nodule, wherein the patient having malignant lung nodule may indicate that the patient has lung cancer, and patient having benign lung nodule may indicate that the patient does not have lung cancer. The machine-learning model can be trained according to a method described herein, e.g. according to the methods of training of the machine learning model of step b′.

In another aspect, the present disclosure provides a computer system for assessing a lung nodule of a subject, comprising: a database or other suitable data storage system that is configured to store a data set; and one or more computer processors operatively coupled to the database, wherein the one or more computer processors are individually or collectively programmed to: (i) analyze the data set to classify the lung nodule of the subject as a malignant lung nodule or a benign lung nodule; (ii) electronically output a report indicative of the classification of the lung nodule of the subject as the malignant lung nodule or the benign lung nodule. Computer-implemented methods as described herein may be executed on computer systems such as those described above. For example, a computer system may comprise one or more processors and one or more memory units that collectively store computer-readable executable instructions that, as a result of execution, cause the one or more processors to collectively perform the programmed steps described above. A computer system as described herein may comprise an assay device communicatively coupled to a personal computer. The data set can be a data set described herein. In some embodiments, the dataset comprise a) gene expression data, wherein the gene expression data is obtained by assaying a biological sample obtained or derived from the subject to produce gene expression measurements of the biological sample from each of a plurality of lung disease-associated genomic loci, wherein the plurality of disease-associated genomic loci comprises at least one gene selected from the group of genes listed in Table 4. In some embodiments, the data set contains i) gene expression measurements of a biological sample from the subject of at least two lung disease-associated genes selected from the group of genes listed in any one or more of Tables 1, 2, 3, 4, 5, 7, and 8, and ii) optionally clinical characteristics data of one or more clinical characteristics of the subject selected from the group of clinical characteristics listed in Table 6. In some embodiments, the data set contains i) gene expression measurements of the biological sample of the subject of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease associated genes selected from the group of genes listed in Table 7, and ii) clinical characteristics data of 1, 2, 3, 4, 5, 6, 7 or 8 clinical characteristics of the subject selected from the group of clinical characteristics listed in Table 6. In some embodiments, the data set contains i) gene expression measurements of the biological sample of the subject of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease-associated genes selected from the group of genes listed in Table 7, and ii) clinical characteristics data of the clinical characteristics of the subject selected from size of the nodule, age of the subject, presence of the nodule in the lung upper lobe, or any combination thereof. The biological sample can be a biological sample described herein. In some embodiments, the data set comprises i) the 31 genes listed in Table 7, and ii) the one or more clinical characteristics of the subject selected from the size of the nodule, age of the patient, and the presence of the nodule in the lung upper lobe. In some embodiments, the data set consists of i) the 31 genes listed in Table 7, and ii) the one or more clinical characteristics of the subject selected from the size of the nodule, age of the patient, and the presence of the nodule in the lung upper lobe. The biological sample can be a biological sample described herein.

In some embodiments, the computer system further comprises an electronic display operatively coupled to the one or more computer processors, wherein the electronic display comprises a graphical user interface that is configured to display the report.

In another aspect, the present disclosure provides one or more non-transitory computer readable media collectively comprising machine-executable code that, upon execution by one or more computer processors, causes the one or more computer processors to perform a method for assessing a lung nodule of a subject, the method comprising: (a) assaying a biological sample obtained or derived from the subject to produce a data set; (b) analyzing the data set to classify the lung nodule of the subject as a malignant lung nodule or a benign lung nodule; and (c) electronically outputting a report indicative of the classification of the lung nodule of the subject as the malignant lung nodule or the benign lung nodule. The data set can be a data set described herein. In some embodiments, the dataset comprise gene expression measurements of the biological sample from each of a plurality of lung disease-associated genomic loci, wherein the plurality of disease-associated genomic loci comprises at least one gene selected from the group listed in Table 4. In some embodiments, the data set contains i) gene expression measurements of the biological sample from the subject of at least two lung disease-associated genes selected from the group of genes listed in any one or more of Tables 1, 2, 3, 4, 5, 7, and 8, and ii) optionally clinical characteristics data of one or more clinical characteristics of the subject selected from the group of clinical characteristics listed in Table 6. In some embodiments, the data set contains i) gene expression measurements of the biological sample of the subject of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease associated genes selected from the group of genes listed in Table 7, and ii) clinical characteristics data of 1, 2, 3, 4, 5, 6, 7 or 8 clinical characteristics of the subject selected from the group of clinical characteristics listed in Table 6. In some embodiments, the data set contains i) gene expression measurements of the biological sample of the subject of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease-associated genes selected from the group of genes listed in Table 7, and ii) clinical characteristics data of the clinical characteristics of the subject selected from size of the nodule, age of the subject, presence of the nodule in the lung upper lobe, or any combination thereof. In some embodiments, the data set comprises i) the 31 genes listed in Table 7, and ii) the one or more clinical characteristics of the subject selected from the size of the nodule, age of the patient, and the presence of the nodule in the lung upper lobe. In some embodiments, the data set consists of i) the 31 genes listed in Table 7, and ii) the one or more clinical characteristics of the subject selected from the size of the nodule, age of the patient, and the presence of the nodule in the lung upper lobe. The biological sample can be a biological sample described herein.

The disclosure includes the use of any inventive method, system, or other composition described herein, including a gene set determined using the inventive methods, for diagnosing a cancer, or for determining and/or administering a treatment of a patient or subject having a cancer.

The current disclosure includes the following aspects

Aspect 1, is directed to a method for assessing a lung nodule of a subject, comprising:
(a) assaying a biological sample obtained or derived from the subject to produce a data set comprising gene expression measurements of the biological sample at each of a plurality of lung disease-associated genomic loci, wherein the plurality of disease-associated genomic loci comprises at least one gene selected from the group listed in in any one or more of Tables 1, 2, 3, 4, 5, 7, and 8;
(b) analyzing the data set to classify the lung nodule of the subject as a malignant lung nodule or a benign lung nodule; and
(c) electronically outputting a report indicative of the classification of the lung nodule of the subject as the malignant lung nodule or the benign lung nodule.

Aspect 2 is directed to the method of aspect 1, wherein the plurality of disease-associated genomic loci comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295 genes selected from the group listed in Table 4.

Aspect 3 is directed to the method of aspect 1 or 2, further comprising classifying the lung nodule of the subject as the malignant lung nodule or the benign lung nodule with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 4 is directed to the method of any one of aspects 1 to 3, further comprising classifying the lung nodule of the subject as the malignant lung nodule or the benign lung nodule with an sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 5 is directed to the method of any one of aspects 1 to 4, further comprising classifying the lung nodule of the subject as the malignant lung nodule or the benign lung nodule with an specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 6 is directed to the method of any one of aspects 1 to 5, further comprising classifying the lung nodule of the subject as the malignant lung nodule or the benign lung nodule with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 7 is directed to the method of any one of aspects 1 to 6, further comprising classifying the lung nodule of the subject as the malignant lung nodule or the benign lung nodule with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 8 is directed to the method of any one of aspects 1 to 7, further comprising classifying the lung nodule of the subject as the malignant lung nodule or the benign lung nodule with an Area-Under-Curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

Aspect 9 is directed to the method of any one of aspects 1 to 8, wherein the subject has a lung cancer.

Aspect 10 is directed to the method of any one of aspects 1 to 8, wherein the subject is suspected of having a lung cancer.

Aspect 11 is directed to the method of any one of aspects 1 to 8, wherein the subject is at elevated risk of having a lung cancer.

Aspect 12 is directed to the method of any one of aspects 1 to 8, wherein the subject is asymptomatic for a lung cancer.

Aspect 13 is directed to the method of any one of aspects 1 to 12 further comprising administering a treatment to the subject based at least in part on the classification of the lung nodule of the subject as the malignant lung nodule or the benign lung nodule.

Aspect 14 is directed to the method of aspect 13, wherein the treatment is configured to treat a lung cancer of the subject.

Aspect 15 is directed to the method of aspect 13, wherein the treatment is configured to reduce a severity of a lung cancer of the subject.

Aspect 16 is directed to the method of aspect 13, wherein the treatment is configured to reduce a risk of having a lung cancer of the subject.

Aspect 17 is directed to the method of aspect 13, wherein the treatment is selected from the group consisting of: surgery, chemotherapy, targeted therapy, immunotherapy, radiotherapy, and any combination thereof.

Aspect 18 is directed to the method of aspect 1, wherein (b) comprises using a trained machine learning classifier to analyze the data set to classify the lung nodule of the subject as the malignant lung nodule or the benign lung nodule.

Aspect 19 is directed to the method of aspect 18, wherein the trained machine learning classifier is trained using gene expression data obtained by a data analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, and a Gene Set Variation Analysis (GSVA) tool (e.g., P-Scope).

Aspect 20 is directed to the method of aspect 18, wherein the trained machine learning classifier is selected from the group consisting of a linear regression, a logistic regression, a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, and a combination thereof.

Aspect 21 is directed to the method of aspect 20, wherein the trained machine learning classifier comprises the logistic regression.

Aspect 22 is directed to the method of aspect 20, wherein the trained machine learning classifier comprises the GLM.

Aspect 23 is directed to the method of aspect 20, wherein the trained machine learning classifier comprises the kNN.

Aspect 24 is directed to the method of aspect 20, wherein the trained machine learning classifier comprises the SVM.

Aspect 25 is directed to the method of aspect 20, wherein the trained machine learning classifier comprises the GBM.

Aspect 26 is directed to the method of aspect 20, wherein the trained machine learning classifier comprises the RF.

Aspect 27 is directed to the method of aspect 20, wherein the trained machine learning classifier comprises the NB.

Aspect 28 is directed to the method of aspect 20, wherein the trained machine learning classifier comprises the EN regression.

Aspect 29 is directed to the method of aspect 1, wherein (b) comprises comparing the data set to a reference data set.

Aspect 30 is directed to the method of aspect 29, wherein the reference data set comprises gene expression measurements of reference biological samples at each of the plurality of lung disease-associated genomic loci.

Aspect 31 is directed to the method of aspect 29, wherein the reference biological samples comprise a first plurality of biological samples obtained or derived from subjects having a malignant lung nodule and a second plurality of biological samples obtained or derived from subjects having a benign lung nodule.

Aspect 32 is directed to the method of any one of aspects 1 to 31, wherein the biological sample is selected from the group consisting of: a blood sample, isolated peripheral blood mononuclear cells (PBMCs), a lung biopsy sample, or any derivative thereof.

Aspect 33 is directed to the method of any one of aspects 1 to 32, further comprising determining a likelihood of the classification of the lung nodule of the subject as the malignant lung nodule or the benign lung nodule.

Aspect 34 is directed to the method of any one of aspects 1 to 33, further comprising monitoring the lung nodule of the subject, wherein the monitoring comprises assessing the lung nodule of the subject at a plurality of time points.

Aspect 35 is directed to the method of aspect 34, wherein a difference in the assessment of the lung nodule of the subject among the plurality of time points is indicative of one or more clinical indications selected from the group consisting of: (i) a diagnosis of the lung nodule of the subject, (ii) a prognosis of the lung nodule of the subject, and (iii) an efficacy or non-efficacy of a course of treatment for treating the lung nodule of the subject.

Aspect 36 is directed to a computer system for assessing a lung nodule of a subject, comprising: a database that is configured to store a dataset comprising gene expression data, wherein the gene expression data is obtained by assaying a biological sample obtained or derived from the subject to produce gene expression measurements of the biological sample at each of a plurality of lung disease-associated genomic loci, wherein the plurality of disease-associated genomic loci comprises at least one gene selected from the group of genes listed in any one or more of Tables 1, 2, 3, 4, 5, 7, and 8; and one or more computer processors operatively coupled to the database, wherein the one or more computer processors are individually or collectively programmed to: (i) analyze the data set to classify the lung nodule of the subject as a malignant lung nodule or a benign lung nodule; (ii) electronically output a report indicative of the classification of the lung nodule of the subject as the malignant lung nodule or the benign lung nodule.

Aspect 37 is directed to the computer system of aspect 36, further comprising an electronic display operatively coupled to the one or more computer processors, wherein the electronic display comprises a graphical user interface that is configured to display the report.

Aspect 38 is directed to one or more non-transitory computer readable media collectively comprising machine-executable code that, upon execution by one or more computer processors, implements a method for assessing a lung nodule of a subject, the method comprising:
(a) assaying a biological sample obtained or derived from the subject to produce a data set comprising gene expression measurements of the biological sample at each of a plurality of lung disease-associated genomic loci, wherein the plurality of disease-associated genomic loci comprises at least one gene selected from the group of genes listed in any one or more of Tables 1, 2, 3, 4, 5, 7, and 8;
(b) analyzing the data set to classify the lung nodule of the subject as a malignant lung nodule or a benign lung nodule; and
(c) electronically outputting a report indicative of the classification of the lung nodule of the subject as the malignant lung nodule or the benign lung nodule.

Aspect 39 is directed to a method for assessing a lung nodule of a patient, the method comprising:
a) obtaining a data set comprising i) gene expression measurements of a biological sample from the patient, of at least two lung disease-associated genes selected from the group of genes listed in any one or more of Tables 1, 2, 3, 4, 5, 7, and 8, and ii) optionally clinical characteristics data of one or more clinical characteristics of the patient, selected from the group of clinical characteristics listed in Table 6, wherein the biological sample is a blood sample, isolated peripheral blood mononuclear cells (PBMCs), or any derivative thereof;
b) providing the data set as an input to a machine-learning model trained to generate an inference of whether the data set is indicative of a malignant lung nodule or a benign lung nodule;
c) receiving, as an output of the machine-learning model, the inference indicating whether the data set is indicative of the malignant lung nodule or the benign lung nodule; and
d) electronically outputting a report classifying the lung nodule of the patient as the malignant lung nodule or the benign lung nodule.

In some embodiments, the data set of Aspect 39, comprises gene expression measurements of the biological sample from the patient, of at least two lung disease-associated genes selected from the group of genes listed in Table 1. In some embodiments, the data set of Aspect 39, comprises gene expression measurements of the biological sample from the patient, of at least two lung disease-associated genes selected from the group of genes listed in Table 2. In some embodiments, the data set of Aspect 39, comprises gene expression measurements of the biological sample from the patient, of at least two lung disease-associated genes selected from the group of genes listed in Table 3. In some embodiments, the data set of Aspect 39, comprises gene expression measurements of the biological sample from the patient, of at least two lung disease-associated genes selected from the group of genes listed in Table 4. In some embodiments, the data set of Aspect 39, comprises gene expression measurements of the biological sample from the patient, of at least two lung disease-associated genes selected from the group of genes listed in Table 5. In some embodiments, the data set of Aspect 39, comprises gene expression measurements of the biological sample from the patient, of at least two lung disease-associated genes selected from the group of genes listed in Table 7. In some embodiments, the data set of Aspect 39, comprises gene expression measurements of the biological sample from the patient, of at least two lung disease-associated genes selected from the group of genes listed in Table 8. In some embodiments, the data set of Aspect 39, comprises i) gene expression measurements of the biological sample from the patient, of at least two lung disease-associated genes selected from the group of genes listed in Table 1, and ii) clinical characteristics data of one or more clinical characteristics of the patient, selected from the group of clinical characteristics listed in Table 6. In some embodiments, the data set of Aspect 39, comprises i) gene expression measurements of the biological sample from the patient, of at least two lung disease-associated genes selected from the group of genes listed in Table 2, and ii) clinical characteristics data of one or more clinical characteristics of the patient, selected from the group of clinical characteristics listed in Table 6. In some embodiments, the data set of Aspect 39, comprises i) gene expression measurements of the biological sample from the patient, of at least two lung disease-associated genes selected from the group of genes listed in Table 3, and ii) clinical characteristics data of one or more clinical characteristics of the patient, selected from the group of clinical characteristics listed in Table 6. In some embodiments, the data set of Aspect 39, comprises i) gene expression measurements of the biological sample from the patient, of at least two lung disease-associated genes selected from the group of genes listed in Table 4, and ii) clinical characteristics data of one or more clinical characteristics of the patient, selected from the group of clinical characteristics listed in Table 6. In some embodiments, the data set of Aspect 39, comprises i) gene expression measurements of the biological sample from the patient, of at least two lung disease-associated genes selected from the group of genes listed in Table 5, and ii) clinical characteristics data of one or more clinical characteristics of the patient, selected from the group of clinical characteristics listed in Table 6. In some embodiments, the data set of Aspect 39, comprises i) gene expression measurements of the biological sample from the patient, of at least two lung disease-associated genes selected from the group of genes listed in Table 7, and ii) clinical characteristics data of one or more clinical characteristics of the patient, selected from the group of clinical characteristics listed in Table 6. In some embodiments, the data set of Aspect 39, comprises i) gene expression measurements of the biological sample from the patient, of at least two lung disease-associated genes selected from the group of genes listed in Table 8, and ii) clinical characteristics data of one or more clinical characteristics of the patient, selected from the group of clinical characteristics listed in Table 6.

Aspect 40 is directed to the method of aspect 39, wherein the at least two lung disease-associated genes are selected from the group of genes listed in Table 7.

Aspect 41 is directed to the method of aspects 39 or 40, wherein the one or more clinical characteristics comprises size of the nodule, age of the patient, and presence of the nodule in the lung upper lobe.

Aspect 42 is directed to the method of any one of aspects 39 to 41, wherein the machine-learning model is developed using a linear regression, a logistic regression (LOG), a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, a linear discriminant analysis (LDA), a decision tree learning (DTREE), an adaptive boosting (ADB), or any combination thereof.

Aspect 43 is directed to the method of any one of aspects 39 to 42, wherein the patient has lung cancer.

Aspect 44 is directed to the method of any one of aspects 39 to 42, wherein the patient does not have lung cancer.

Aspect 45 is directed to the method of any one of aspects 39 to 42, wherein the patient is at an elevated risk of having lung cancer.

Aspect 46 is directed to the method of any one of aspects 39 to 43 and 45, wherein the patient is asymptomatic for lung cancer.

Aspect 47 is directed to the method of any one of aspects 39 to 43, 45 and 46, further comprising administering a treatment based on the patient's nodule being classified as a malignant nodule.

Aspect 48 is directed to the method of aspect 47, wherein the treatment is surgery, chemotherapy, targeted therapy, immunotherapy, radiotherapy, or any combination thereof.

Aspect 49 is directed to the method of any one of aspects 39 to 48, wherein the inference includes a confidence value between 0 and 1 that the lung nodule is malignant.

Aspect 50 is directed to the method of any one of aspects 39 to 49, wherein the at least two lung disease-associated genes comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295, genes selected from the group of genes listed in Table 4.

Aspect 51 is directed to the method of any one of aspects 39 to 50, wherein the at least two lung disease-associated genes comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31, genes selected from the group of genes listed in Table 7.

Aspect 52 is directed to the method of any one of aspects 39 to 51, comprising classifying the lung nodule of the patient as the malignant lung nodule or the benign lung nodule with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 53 is directed to the method of any one of aspects 39 to 52, comprising classifying the lung nodule of the patient as the malignant lung nodule or the benign lung nodule with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 54 is directed to the method of any one of aspects 39 to 53, comprising classifying the lung nodule of the patient as the malignant lung nodule or the benign lung nodule with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 55 is directed to the method of any one of aspects 39 to 54, comprising classifying the lung nodule of the patient as the malignant lung nodule or the benign lung nodule with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 56 is directed to the method of any one of aspects 39 to 55, comprising classifying the lung nodule of the patient as the malignant lung nodule or the benign lung nodule with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 57 is directed to the method of any one of aspects 39 to 56, wherein the trained machine learning model has a receiver operating characteristic (ROC) curve with an Area-Under-Curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

Aspect 58 is directed to a system for assessing a lung module of a patient, the system comprising:
one or more processors; and
one or more memories storing executable instructions that, as a result of execution by the one or more processors, cause the system to:
obtain, from a data base, a data set comprising i) gene expression measurements of a biological sample of a patient of a plurality of lung disease-associated genes, selected from the group of genes listed in any one or more of Tables 1, 2, 3, 4, 5, 7, and 8, and ii) optionally clinical characteristics data of one or more clinical characteristics of the patient, selected from the group of clinical characteristics listed in Table 6, wherein the biological sample is a blood sample, isolated peripheral blood mononuclear cells (PBMCs), or any derivative thereof;
provide the dataset as an input to a machine-learning model trained to generate an inference of whether the data set is indicative of a malignant lung nodule or a benign lung nodule;
receive, as an output of the machine-learning model, the inference indicating whether the composite data set is indicative of the malignant lung nodule or the benign lung nodule; and
generate a report classifying the lung nodule of the patient as the malignant lung nodule or the benign lung nodule.

In some embodiments, the data set of Aspect 58, comprises gene expression measurements of the biological sample from the patient, of at least two lung disease-associated genes selected from the group of genes listed in Table 1. In some embodiments, the data set of Aspect 58, comprises gene expression measurements of the biological sample from the patient, of at least two lung disease-associated genes selected from the group of genes listed in Table 2. In some embodiments, the data set of Aspect 58, comprises gene expression measurements of the biological sample from the patient, of at least two lung disease-associated genes selected from the group of genes listed in Table 3. In some embodiments, the data set of Aspect 58, comprises gene expression measurements of the biological sample from the patient, of at least two lung disease-associated genes selected from the group of genes listed in Table 4. In some embodiments, the data set of Aspect 58, comprises gene expression measurements of the biological sample from the patient, of at least two lung disease-associated genes selected from the group of genes listed in Table 5. In some embodiments, the data set of Aspect 58, comprises gene expression measurements of the biological sample from the patient, of at least two lung disease-associated genes selected from the group of genes listed in Table 7. In some embodiments, the data set of Aspect 58, comprises gene expression measurements of the biological sample from the patient, of at least two lung disease-associated genes selected from the group of genes listed in Table 8. In some embodiments, the data set of Aspect 58, comprises i) gene expression measurements of the biological sample from the patient, of at least two lung disease-associated genes selected from the group of genes listed in Table 1, and ii) clinical characteristics data of one or more clinical characteristics of the patient, selected from the group of clinical characteristics listed in Table 6. In some embodiments, the data set of Aspect 58, comprises i) gene expression measurements of the biological sample from the patient, of at least two lung disease-associated genes selected from the group of genes listed in Table 2, and ii) clinical characteristics data of one or more clinical characteristics of the patient, selected from the group of clinical characteristics listed in Table 6. In some embodiments, the data set of Aspect 58, comprises) gene expression measurements of the biological sample from the patient, of at least two lung disease-associated genes selected from the group of genes listed in Table 3, and ii) clinical characteristics data of one or more clinical characteristics of the patient, selected from the group of clinical characteristics listed in Table 6. In some embodiments, the data set of Aspect 58, comprises i) gene expression measurements of the biological sample from the patient, of at least two lung disease-associated genes selected from the group of genes listed in Table 4, and ii) clinical characteristics data of one or more clinical characteristics of the patient, selected from the group of clinical characteristics listed in Table 6. In some embodiments, the data set of Aspect 58, comprises i) gene expression measurements of the biological sample from the patient, of at least two lung disease-associated genes selected from the group of genes listed in Table 5, and ii) clinical characteristics data of one or more clinical characteristics of the patient, selected from the group of clinical characteristics listed in Table 6. In some embodiments, the data set of Aspect 58, comprises i) gene expression measurements of the biological sample from the patient, of at least two lung disease-associated genes selected from the group of genes listed in Table 7, and ii) clinical characteristics data of one or more clinical characteristics of the patient, selected from the group of clinical characteristics listed in Table 6. In some embodiments, the data set of Aspect 58, comprises i) gene expression measurements of the biological sample from the patient, of at least two lung disease-associated genes selected from the group of genes listed in Table 8, and ii) clinical characteristics data of one or more clinical characteristics of the patient, selected from the group of clinical characteristics listed in Table 6.

Aspect 59 is directed to a non-transitory computer-readable medium storing executable instructions for assessing a lung nodule of a patient that, as a result of execution by one or more processors of a computer system, cause the computer system to:
obtain, from a data base, a data set comprising i) gene expression measurements of a biological sample of a patient of a plurality of lung disease-associated genes, selected from the group of genes listed in any one or more of Tables 1, 2, 3, 4, 5, 7, and 8, and ii) optionally clinical characteristics data of one or more clinical characteristics of the patient, selected from the group of clinical characteristics listed in Table 6, wherein the biological sample is a blood sample, isolated peripheral blood mononuclear cells (PBMCs), or any derivative thereof;
provide the data set as an input to a machine-learning model trained to generate an inference of whether the data set is indicative of a malignant lung nodule or a benign lung nodule;
receive, as an output of the machine-learning model, the inference indicating whether the composite data set is indicative of the malignant lung nodule or the benign lung nodule; and
generate a report classifying the lung nodule of the patient as the malignant lung nodule or the benign lung nodule.

In some embodiments, the data set of Aspect 59, comprises gene expression measurements of the biological sample from the patient, of at least two lung disease-associated genes selected from the group of genes listed in Table 1. In some embodiments, the data set of Aspect 59, comprises gene expression measurements of the biological sample from the patient, of at least two lung disease-associated genes selected from the group of genes listed in Table 2. In some embodiments, the data set of Aspect 59, comprises gene expression measurements of the biological sample from the patient, of at least two lung disease-associated genes selected from the group of genes listed in Table 3. In some embodiments, the data set of Aspect 59, comprises gene expression measurements of the biological sample from the patient, of at least two lung disease-associated genes selected from the group of genes listed in Table 4. In some embodiments, the data set of Aspect 59, comprises gene expression measurements of the biological sample from the patient, of at least two lung disease-associated genes selected from the group of genes listed in Table 5. In some embodiments, the data set of Aspect 59, comprises gene expression measurements of the biological sample from the patient, of at least two lung disease-associated genes selected from the group of genes listed in Table 7. In some embodiments, the data set of Aspect 59, comprises gene expression measurements of the biological sample from the patient, of at least two lung disease-associated genes selected from the group of genes listed in Table 8. In some embodiments, the data set of Aspect 59, comprises i) gene expression measurements of the biological sample from the patient, of at least two lung disease-associated genes selected from the group of genes listed in Table 1, and ii) clinical characteristics data of one or more clinical characteristics of the patient, selected from the group of clinical characteristics listed in Table 6. In some embodiments, the data set of Aspect 59, comprises i) gene expression measurements of the biological sample from the patient, of at least two lung disease-associated genes selected from the group of genes listed in Table 2, and ii) clinical characteristics data of one or more clinical characteristics of the patient, selected from the group of clinical characteristics listed in Table 6. In some embodiments, the data set of Aspect 59, comprises i) gene expression measurements of the biological sample from the patient, of at least two lung disease-associated genes selected from the group of genes listed in Table 3, and ii) clinical characteristics data of one or more clinical characteristics of the patient, selected from the group of clinical characteristics listed in Table 6. In some embodiments, the data set of Aspect 59, comprises i) gene expression measurements of the biological sample from the patient, of at least two lung disease-associated genes selected from the group of genes listed in Table 4, and ii) clinical characteristics data of one or more clinical characteristics of the patient, selected from the group of clinical characteristics listed in Table 6. In some embodiments, the data set of Aspect 59, comprises i) gene expression measurements of the biological sample from the patient, of at least two lung disease-associated genes selected from the group of genes listed in Table 5, and ii) clinical characteristics data of one or more clinical characteristics of the patient, selected from the group of clinical characteristics listed in Table 6. In some embodiments, the data set of Aspect 59, comprises i) gene expression measurements of the biological sample from the patient, of at least two lung disease-associated genes selected from the group of genes listed in Table 7, and ii) clinical characteristics data of one or more clinical characteristics of the patient, selected from the group of clinical characteristics listed in Table 6. In some embodiments, the data set of Aspect 59, comprises i) gene expression measurements of the biological sample from the patient, of at least two lung disease-associated genes selected from the group of genes listed in Table 8, and ii) clinical characteristics data of one or more clinical characteristics of the patient, selected from the group of clinical characteristics listed in Table 6.

Aspect 60 is directed a method for determining a gene set capable of classifying a lung nodule benign or malignant without performing biopsy, the method comprising:
obtaining a reference data set comprising a plurality of individual reference data sets, wherein a respective individual reference data set of the plurality of individual reference data sets comprises i) gene expression measurements of a plurality of genes of a reference biological sample from a reference subject having a lung nodule, ii) data regarding whether the lung nodule of the reference subject is benign or malignant, and iii) optionally clinical characteristics data of one or more clinical characteristics selected from the group of clinical characteristics listed in Table 6 of the reference subject, wherein the reference biological sample is a blood sample, isolated peripheral blood mononuclear cells (PBMCs), or any derivative thereof;
training a machine learning model using the reference data set, wherein the machine learning model is trained to infer whether a lung nodule is benign or malignant based at least in part on one or more predictors selected from the plurality of genes, and optionally the one or more clinical characteristics;
determining feature importance values of the plurality of genes; and
determining the gene set based at least in part on the feature importance values.

In some embodiments, the respective individual reference data set of Aspect 60, comprises i) gene expression measurements of the plurality of genes of the reference biological sample, and ii) data regarding whether the lung nodule of the reference subject is benign or malignant; and the machine learning model is trained to infer whether a lung nodule is benign or malignant based at least in part on one or more predictors selected from the plurality of genes. In some embodiments, the respective individual reference data set of Aspect 60, comprises i) gene expression measurements of the plurality of genes of the reference biological sample, ii) data regarding whether the lung nodule of the reference subject is benign or malignant, and iii) optionally clinical characteristics data of one or more clinical characteristics selected from the group of clinical characteristics listed in Table 6 of the reference subject; and the machine learning model is trained to infer whether a lung nodule is benign or malignant based at least in part on one or more predictors selected from the plurality of genes and the one or more clinical characteristics.

Aspect 61 is directed to the method of aspect 60, wherein the plurality of genes comprises at least 2 genes selected from the group of genes listed in Table 9.

Aspect 62 is directed a method for developing a trained machine learning model capable of inferring whether a lung nodule of a patient is benign or malignant, the method comprising:
(a) obtaining a first reference data set comprising a plurality of first individual reference data sets, wherein a respective first individual reference data set of the plurality of first individual reference data sets comprises i) gene expression measurements of a plurality of genes of a reference biological sample from a reference subject having a lung nodule, ii) data regarding whether the lung nodule of the reference subject is benign or malignant, and iii) optionally clinical characteristics data of one or more clinical characteristics selected from a group of clinical characteristics listed in Table 6 of the reference subject, wherein the biological sample is a blood sample, isolated peripheral blood mononuclear cells (PBMCs), or any derivative thereof;
(b) training a first machine learning model using the first reference data set, wherein the first machine learning model is trained to infer whether a lung nodule is benign or malignant, based at least in part on one or more predictors selected from the plurality of genes, and optionally the one or more clinical characteristics;
(c) determining feature importance values of the one or more predictors of the first machine learning model;
(d) selecting A predictors of the first machine learning model based at least in part on the feature importance values, wherein A is an integer from 5 to 2000; and
(e) training a second machine learning model based at least in part on a second reference data set comprising a plurality of second individual reference data sets, wherein a respective second individual reference data set of the plurality of second individual reference data sets comprises i) measurement data of the A predictors of the reference subject, and ii) data regarding whether the lung nodule of the reference subject is benign or malignant, to obtain the trained machine learning model, wherein the trained machine learning model is trained to infer whether a lung nodule is benign or malignant, based at least in part on measurement data of the A predictors.

In some embodiments, the respective first individual reference data set of Aspect 62, comprises i) gene expression measurements of the plurality of genes of the reference biological sample, and ii) data regarding whether the lung nodule of the reference subject is benign or malignant; and the first machine learning model is trained to infer whether a lung nodule is benign or malignant based at least in part on one or more predictors selected from the plurality of genes. In some embodiments, the respective first individual reference data set of Aspect 60, comprises i) gene expression measurements of the plurality of genes of the reference biological sample, ii) data regarding whether the lung nodule of the reference subject is benign or malignant, and iii) optionally clinical characteristics data of one or more clinical characteristics selected from the group of clinical characteristics listed in Table 6 of the reference subject; and the first machine learning model is trained to infer whether a lung nodule is benign or malignant based at least in part on one or more predictors selected from the plurality of genes and the one or more clinical characteristics.

Aspect 63 is directed to the aspect of 62, wherein the plurality of genes comprises at least 2 genes selected from the group of genes listed in Table 9.

Aspect 64 is directed to the method of any one of aspects 62 to 63, wherein the A predictors have top 5 to 200 feature importance values.

Aspect 65 is directed to the method of any one of aspects 62 to 64, wherein the trained machine learning model has an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 66 is directed to the method of any one of aspects 62 to 65, wherein the trained machine learning model has an sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 67 is directed to the method of any one of aspects 62 to 66, wherein the trained machine learning model has an specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 68 is directed to the method of any one of aspects 62 to 67, wherein the trained machine learning model has a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 69 is directed to the method of any one of aspects 62 to 68, wherein the trained machine learning model has a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 70 is directed to the method of any one of aspects 62 to 69, wherein the trained machine learning model has a receiver operating characteristic (ROC) curve with an Area-Under-Curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

Aspect 71 is directed to the method of any one of aspects 62 to 70, wherein the first machine learning model and second machine learning model is independently trained using a linear regression, a logistic regression (LOG), a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, a linear discriminant analysis (LDA), a decision tree learning (DTREE), an adaptive boosting (ADB), or any combination thereof.

Aspect 72 is directed to a method for assessing a lung nodule of a patient, the method comprising:
(a) obtaining a data set comprising measurement data of the patient of one or more of the A predictors of any one of aspects 62 to 64;
(b) providing the data set as an input to a trained machine-learning model trained according to the methods of any one of claims 62 to 71 to generate an inference of whether the data set is indicative a malignant lung nodule or a benign lung nodule;
(c) receiving, as an output of the machine-learning model, the inference indicating whether the data set is indicative of the malignant lung nodule or the benign lung nodule; and
(d) electronically outputting a report classifying the lung nodule of the patient as the malignant lung nodule or the benign lung nodule.

Aspect 73 is directed to the method of aspect 72, wherein the biological sample is a blood sample, isolated peripheral blood mononuclear cells (PBMCs), or any derivative thereof.

Aspect 74 is directed to the method of any one of aspects 72 to 73, wherein the patient has lung cancer.

Aspect 75 is directed to the method of any one of aspects 72 to 73, wherein the patient does not have lung cancer.

Aspect 76 is directed to the method of any one of aspects 72 to 73, wherein the patient is at elevated risk of having lung cancer.

Aspect 77 is directed to the method of any one of aspects 72 to 74 and 76, wherein the patient is asymptomatic for lung cancer.

Aspect 78 is directed to the method of any one of aspects 72 to 74, 76 and 77, further comprising administering a treatment based on the patient's lung nodule being classified as a malignant nodule.

Aspect 79 is directed to the method of aspect 78, wherein the treatment is surgery, chemotherapy, targeted therapy, immunotherapy, radiotherapy, or any combination thereof.

Aspect 80 is directed to a method for treating lung cancer in a patient having a lung nodule, the method comprising:
(a) obtaining a data set comprising i) gene expression measurements of a biological sample from the patient, of at least two lung disease-associated genes selected from the group of genes listed in any one or more of Tables 1, 2, 3, 4, 5, 7, and 8, and ii) optionally clinical characteristics data of one or more clinical characteristics selected from the group of clinical characteristics listed in Table 6 of the patient, wherein the biological sample is a blood sample, isolated peripheral blood mononuclear cells (PBMCs), or any derivative thereof
(b) providing the data set as an input to a trained machine-learning model trained to generate an inference of whether the data set is indicative of a malignant lung nodule or a benign lung nodule;
(c) receiving, as an output of the machine-learning model, the inference indicating whether the data set is indicative of the malignant lung nodule or the benign lung nodule; and
(d) administering a treatment based on the patient's lung nodule being classified as the malignant lung nodule.

In some embodiments, the data set of Aspect 80, comprises gene expression measurements of the biological sample from the patient, of at least two lung disease-associated genes selected from the group of genes listed in Table 1. In some embodiments, the data set of Aspect 80, comprises gene expression measurements of the biological sample from the patient, of at least two lung disease-associated genes selected from the group of genes listed in Table 2. In some embodiments, the data set of Aspect 80, comprises gene expression measurements of the biological sample from the patient, of at least two lung disease-associated genes selected from the group of genes listed in Table 3. In some embodiments, the data set of Aspect 80, comprises gene expression measurements of the biological sample from the patient, of at least two lung disease-associated genes selected from the group of genes listed in Table 4. In some embodiments, the data set of Aspect 80, comprises gene expression measurements of the biological sample from the patient, of at least two lung disease-associated genes selected from the group of genes listed in Table 5. In some embodiments, the data set of Aspect 80, comprises gene expression measurements of the biological sample from the patient, of at least two lung disease-associated genes selected from the group of genes listed in Table 7. In some embodiments, the data set of Aspect 80, comprises gene expression measurements of the biological sample from the patient, of at least two lung disease-associated genes selected from the group of genes listed in Table 8. In some embodiments, the data set of Aspect 80, comprises i) gene expression measurements of the biological sample from the patient, of at least two lung disease-associated genes selected from the group of genes listed in Table 1, and ii) clinical characteristics data of one or more clinical characteristics of the patient, selected from the group of clinical characteristics listed in Table 6. In some embodiments, the data set of Aspect 80, comprises i) gene expression measurements of the biological sample from the patient, of at least two lung disease-associated genes selected from the group of genes listed in Table 2, and ii) clinical characteristics data of one or more clinical characteristics of the patient, selected from the group of clinical characteristics listed in Table 6. In some embodiments, the data set of Aspect 80, comprises i) gene expression measurements of the biological sample from the patient, of at least two lung disease-associated genes selected from the group of genes listed in Table 3, and ii) clinical characteristics data of one or more clinical characteristics of the patient, selected from the group of clinical characteristics listed in Table 6. In some embodiments, the data set of Aspect 80, comprises i) gene expression measurements of the biological sample from the patient, of at least two lung disease-associated genes selected from the group of genes listed in Table 4, and ii) clinical characteristics data of one or more clinical characteristics of the patient, selected from the group of clinical characteristics listed in Table 6. In some embodiments, the data set of Aspect 80, comprises i) gene expression measurements of the biological sample from the patient, of at least two lung disease-associated genes selected from the group of genes listed in Table 5, and ii) clinical characteristics data of one or more clinical characteristics of the patient, selected from the group of clinical characteristics listed in Table 6. In some embodiments, the data set of Aspect 80, comprises i) gene expression measurements of the biological sample from the patient, of at least two lung disease-associated genes selected from the group of genes listed in Table 7, and ii) clinical characteristics data of one or more clinical characteristics of the patient, selected from the group of clinical characteristics listed in Table 6. In some embodiments, the data set of Aspect 80, comprises i) gene expression measurements of the biological sample from the patient, of at least two lung disease-associated genes selected from the group of genes listed in Table 8, and ii) clinical characteristics data of one or more clinical characteristics of the patient, selected from the group of clinical characteristics listed in Table 6.

Aspect 81 is directed to the method of aspect 80, wherein the at least two lung disease-associated genes are selected from the group of genes listed in Table 7.

Aspect 82 is directed to the method of aspects 80 or 81, wherein the one or more clinical characteristics comprises size of the nodule, age of the patient, and presence of the nodule in the lung upper lobe.

Aspect 83 is directed to the method of any one of aspects 80 to 82, wherein the machine-learning model is developed using a linear regression, a logistic regression (LOG), a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, a linear discriminant analysis (LDA), a decision tree learning (DTREE), an adaptive boosting (ADB), or any combination thereof.

Aspect 84 is directed to the method of any one of aspects 80 to 83, wherein the treatment is surgery, chemotherapy, targeted therapy, immunotherapy, radiotherapy, or any combination thereof.

Aspect 85 is directed to the method of any one of aspects 80 to 84, wherein the inference includes a confidence value between 0 and 1 that the lung nodule is malignant.

Aspect 86 is directed to the method of any one of aspects 80 to 85, wherein the at least two lung disease-associated genes comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295, genes selected from the group of genes listed in Table 4.

Aspect 87 is directed to the method of any one of aspects 80 to 86, wherein the at least two lung disease-associated genes comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31, genes selected from the group of genes listed in Table 7.

Aspect 88 is directed to the method of any one of aspects 80 to 87, comprising classifying the lung nodule of the patient as the malignant lung nodule or the benign lung nodule with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 89 is directed to the method of any one of aspects 80 to 88, comprising classifying the lung nodule of the patient as the malignant lung nodule or the benign lung nodule with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 90 is directed to the method of any one of aspects 80 to 89, comprising classifying the lung nodule of the patient as the malignant lung nodule or the benign lung nodule with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 91 is directed to the method of any one of aspects 80 to 90, comprising classifying the lung nodule of the patient as the malignant lung nodule or the benign lung nodule with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 92 is directed to the method of any one of aspects 80 to 91, comprising classifying the lung nodule of the patient as the malignant lung nodule or the benign lung nodule with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

Aspect 93 is directed to the method of any one of aspects 80 to 92, wherein the trained machine learning model has a receiver operating characteristic (ROC) curve with an Area-Under-Curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A is a receiver operating characteristic (ROC) plot showing performance of eight machine learning classifiers using a set of 1,178 gene features generated from ribonucleic acid (RNA) sequencing (RNA-Seq) data to distinguish malignant lung nodules versus benign lung nodules. The 1,178 genes were differentially expressed in blood samples of patients with malignant lung nodules versus benign lung nodules. The eight machine learning classifiers include LOG, GLM, kNN, RF, SVM, GBM, NB, and EN.

FIG. 1B shows results of exemplary trained machine learning classifier algorithms to analyze RNA Seq data using the set of 1,178 gene features to distinguish malignant lung nodules versus benign lung nodules.

FIG. 2A is a ROC plot for an optimization of differentially expressed genes to distinguish malignant lung nodules versus benign lung nodules based on an analysis of RNA-Seq data. The six machine learning classifiers include LOG, GLM, kNN, RF, SVM, and GBM.

FIG. 2B shows results of exemplary trained machine learning classifier algorithms in the FIG. 2A optimization of differentially expressed genes to distinguish malignant lung nodules versus benign lung nodules.

FIG. 3A is a ROC plot showing performance of eight machine learning classifiers using a set of 182 gene features generated from RNA-Seq data to distinguish malignant lung nodules versus benign lung nodules. The eight machine learning classifiers include LOG, GLM, kNN, RF, SVM, GBM, NB, and EN.

FIG. 3B shows results of exemplary trained machine learning classifier algorithms to analyze RNASeq data using the set of 182 gene features to distinguish malignant lung nodules versus benign lung nodules.

FIG. 4A is a ROC plot showing performance of machine learning classifiers using a set of 182 gene features generated from RNA-Seq data to distinguish malignant lung nodules versus benign lung nodules. The eight machine learning classifiers include LOG, GLM, kNN, RF, SVM, GBM, NB, and EN.

FIG. 4B shows tabulated results of exemplary trained machine learning classifier algorithms corresponding to FIG. 4A.

FIG. 5A is a ROC plot showing performance of eight machine learning classifiers using a set of 175 gene features generated from RNA-Seq data to distinguish malignant lung nodules versus benign lung nodules. The eight machine learning classifiers include LOG, GLM, kNN, RF, SVM, GBM, NB, and EN.

FIG. 5B shows tabulated results of exemplary trained machine learning classifier algorithms corresponding to FIG. 5A.

FIG. 6A is a ROC plot showing performance of machine learning classifiers using a set of 62 gene features generated from RNA-Seq data to distinguish malignant lung nodules versus benign lung nodules. The eight machine learning classifiers include LOG, GLM, kNN, RF, SVM, GBM, NB, and EN.

FIG. 6B shows tabulated results of exemplary trained machine learning classifier algorithms corresponding to FIG. 6A.

FIG. 7A is a ROC plot showing performance of machine learning classifiers using a set of 295 gene features generated from RNA-Seq data to distinguish malignant lung nodules versus benign lung nodules. The eight machine learning classifiers include LOG, GLM, kNN, RF, SVM, GBM, NB, and EN.

FIG. 7B shows tabulated results of exemplary trained machine learning classifier algorithms corresponding to FIG. 7A.

FIG. 8A is a ROC plot showing performance of machine learning classifiers using a set of 175 gene features generated from RNA-Seq data to distinguish malignant lung nodules versus benign lung nodules. The eight machine learning classifiers include LOG, GLM, kNN, RF, SVM, GBM, NB, and EN.

FIG. 8B shows tabulated results of exemplary trained machine learning classifier algorithms corresponding to FIG. 8A.

FIG. 9A is a cumulative fraction of lung nodules predicted by a logistic regression classifier using a set of 175 gene features.

FIG. 9B is a cumulative fraction of lung nodules predicted by a gradient boosting classifier using a set of 175 gene features.

FIG. 10 illustrates an overview of an example method 1000 for assessing a lung nodule of a subject.

FIG. 11 shows a computer system 1101 that is programmed or otherwise configured to implement methods provided herein.

FIG. 12 shows the correlation plot of the 8 clinical characteristics features listed in Table 6.

FIG. 13A-E: FIG. 13A shows ROC plots showing performance of the 9 machine learning classifiers using clinical characteristics data of the 8 clinical characteristics features listed in Table 6, to distinguish malignant lung nodules versus benign lung nodules (in 152 patients). FIG. 13B shows Precision/Recall curve of the 9 machine learning classifiers using clinical characteristics data of the 8 clinical characteristics features (Table 6), to distinguish malignant lung nodules versus benign lung nodules. FIG. 13C shows the tabulated results of the 9 machine learning classifiers corresponding to FIG. 13A. FIG. 13D shows feature importance of the 8 clinical characteristics features (Table 6) for the 9 machine learning classifiers. FIG. 13E shows feature importance of the 8 clinical characteristics features for all the 9 classifiers. The 9 machine learning classifiers are LOG, RF, SVM, DTREE, ADB, NB, LDA, kNN, and GBM.

FIG. 14A-E: FIG. 14A shows ROC plots showing performance of the 9 machine learning classifiers using clinical characteristics data of 4 clinical characteristics features, NCNSZE (nodule size), NCNUPYN (nodule in the upper lobe), AGE, and NCNMYN (Nodule Spiculated), to distinguish malignant lung nodules versus benign lung nodules. FIG. 14B shows Precision/Recall curve of the 9 machine learning classifiers using clinical characteristics data of the 4 clinical characteristics features, to distinguish malignant lung nodules versus benign lung nodules. FIG. 14C shows the tabulated results of the 9 machine learning classifiers corresponding to FIG. 14A. FIG. 14D shows feature importance of the 4 clinical characteristics features for the 9 machine learning classifiers. FIG. 14E shows feature importance of the 4 clinical characteristics features for all the 9 classifiers. The 9 machine learning classifiers are LOG, RF, SVM, DTREE, ADB, NB, LDA, kNN, and GBM.

FIG. 15A-E: FIG. 15A shows ROC plots showing performance of the 9 machine learning classifiers using clinical characteristics data of 9 clinical characteristics features (8 features in Table 6 and cancer history) to distinguish malignant lung nodules versus benign lung nodules. FIG. 15B shows Precision/Recall curve of the 9 machine learning classifiers using clinical characteristics data of the 9 clinical characteristics features, to distinguish malignant lung nodules versus benign lung nodules. FIG. 15C shows the tabulated results of the 9 machine learning classifiers corresponding to FIG. 15A. FIG. 15D shows feature importance of the 9 clinical characteristics features for the 9 machine learning classifiers. FIG. 15E shows feature importance of the 9 clinical characteristics features for all the 9 classifiers. The 9 machine learning classifiers are LOG, RF, SVM, DTREE, ADB, NB, LDA, kNN, and GBM.

FIG. 16A-D: FIG. 16A shows ROC plots showing performance of the 9 machine learning classifiers using gene expression data of the 142 gene features (Table 5), and a clinical characteristics data of 3 clinical features (NCNSZE (nodule size), NCNUPYN (nodule in the upper lobe), and AGE), to distinguish malignant lung nodules versus benign lung nodules. FIG. 16B shows Precision/Recall curve of the 9 machine learning classifiers using gene expression data of the 142 gene features, and a clinical characteristics data of 3 clinical features, to distinguish malignant lung nodules versus benign lung nodules. FIG. 16C shows the tabulated results of the 9 machine learning classifiers corresponding to FIG. 16A. FIG. 16D shows the tabulated results of the 9 machine learning classifiers corresponding to FIG. 16A with oversampling correction applied (e.g. 80 sample with benign lung nodule, and 80 samples with malignant lung nodule). The 9 machine learning classifiers are LOG, RF, SVM, DTREE, ADB, NB, LDA, kNN, and GBM.

FIG. 17A-E: FIG. 17A shows ROC plots showing performance of the 9 machine learning classifiers using measurement data of the 34 predictors (Table 7), to distinguish malignant lung nodules versus benign lung nodules. FIG. 17B shows Precision/Recall curve of the 9 machine learning classifiers using measurement data of the 34 predictors, to distinguish malignant lung nodules versus benign lung nodules. FIG. 17C shows the tabulated results of the machine learning classifiers LOG and RF corresponding to FIG. 17A. FIG. 17D shows the tabulated results of the 9 machine learning classifiers corresponding to FIG. 17A, with oversampling correction applied (e.g. 80 sample with benign lung nodule, and 80 samples with malignant lung nodule). FIG. 17 E shows feature importance of the 34 clinical characteristics features for all the 9 classifier. The 9 machine learning classifiers are LOG, RF, SVM, DTREE, ADB, NB, LDA, kNN, and GBM.

FIG. 18A-C: FIG. 18A shows ROC plots showing performance of the 9 machine learning classifiers using gene expression data of the 175 gene features (Table 2), and a clinical characteristics data of 4 clinical features (NCNSZE (nodule size), NCNUPYN (nodule in the upper lobe), AGE, and NCNMYN (Nodule Spiculated)), to distinguish malignant lung nodules versus benign lung nodules. FIG. 18B shows Precision/Recall curve of the 9 machine learning classifiers using gene expression data of the 175 gene features, and a clinical characteristics data of 4 clinical features, to distinguish malignant lung nodules versus benign lung nodules. FIG. 18C shows the tabulated results of the 9 machine learning classifiers corresponding to FIG. 18A. The 9 machine learning classifiers are LOG, RF, SVM, DTREE, ADB, NB, LDA, kNN, and GBM.

### DETAILED DESCRIPTION

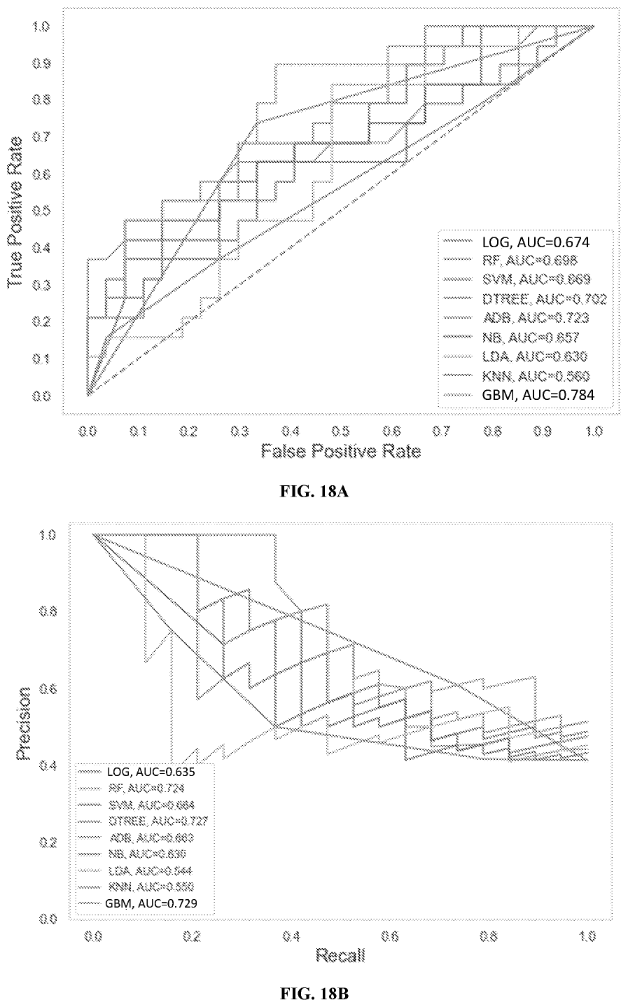

In certain aspects of the current disclosure, methods and systems for assessing a lung nodule of a patient, using machine learning are disclosed. The methods can classify lung nodule as benign or malignant, without performing a biopsy of the nodule. In certain embodiments, a biopsy of the nodule may be performed to confirm, and/or follow-up on the results from machine learning classification. As shown in a non-limiting manner in the Examples, using gene expression measurements of a biological sample from the patient, and optionally clinical characteristics data of the patient, machine learning methods of the current disclosure can classify the nodule. The biological sample can be a blood sample. The methods can have relatively high accuracy, specificity, selectivity, positive predictive value, and/or negative predictive value. Further, as shown in a non-limiting manner in Example 5, it was also found that, using both gene expression data and clinical characteristics data compared to using gene expression data only, predictive power (e.g. accuracy, specificity, selectivity, positive predictive value, and/or negative predictive value) of the machine learning models and the method can be improved. For example, as shown in FIG. 17D, accuracy, specificity, selectivity, above 0.9 can be obtained with certain machine learning models using relatively fewer number of predictors containing gene and clinical characteristics. In certain embodiments, a treatment of lung cancer can be administered based on the results from machine learning classification. One of the potential benefits of certain embodiments of the current disclosures include is that a biopsy can be avoided in cases where the ML classification model outputs a high confidence that a lung nodule is benign or malignant. The benefit here is that in conventional techniques, a biopsy is always performed as it is the only way to determine whether the lung nodule is benign or malignant. However, biopsy procedure carries inherent risks, and the risks for a biopsy may outweigh the benefits for some patients but not others, based on their individual circumstances. The ML model can be used to better inform the clinician of whether the benefits of getting the biopsy outweigh the risks of a biopsy procedure (e.g., we can contrive an example in which a biopsy should be avoided, perhaps where a patient is (1) at heightened risk of complications of a biopsy due to some other health-related condition or the location of the tumor and (2) the blood sample indicates that the lung nodule has high likelihood of being benign or malignant). While most of the scenarios we are working on focus on more accurately identifying instances of malignant lung nodule, the ability to avoid an unnecessary biopsy can also be considered a technical advantage/practical benefit.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description. Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “subject,” or “reference subject”, as used herein, generally refer to a human such as a patient. The subject may be a person (e.g., a patient) with a lung cancer, a benign lung nodule, or a malignant lung nodule; or a person that has been treated for a lung cancer, a benign lung nodule, or a malignant lung nodule; or a person that is being monitored for a lung cancer, a benign lung nodule, or a malignant lung nodule; or a person that is suspected of having a lung cancer, a benign lung nodule, or a malignant lung nodule; or a person that does not have or is not suspected of having a lung cancer, a benign lung nodule, or a malignant lung nodule. The term “patient,” as used herein, generally refers to a human patient. The patient may be a person with a lung cancer, a benign lung nodule, or a malignant lung nodule; or a person that has been treated for a lung cancer, a benign lung nodule, or a malignant lung nodule; or a person that is being monitored for a lung cancer, a benign lung nodule, or a malignant lung nodule; or a person that is suspected of having a lung cancer, a benign lung nodule, or a malignant lung nodule; or a person that does not have or is not suspected of having a lung cancer, a benign lung nodule, or a malignant lung nodule.

The blood sample can be whole blood, blood cells, serum, plasma, or any combination thereof.

Tables 1, 2, 3, 4, 5, and 9 list lung disease-associated gene. Table 7 lists 31 lung disease-associated gene and 3 clinical characteristics. Table 8 lists 21 lung disease-associated gene and 1 clinical characteristics. Table 6 lists 8 clinical characteristics. Tables 1, 2, 3, 4, 5, 6, 7, 8 and 9, and all of contents of the Tables are incorporated as part of specification of this disclosure.

In an aspect, the present disclosure provides a method for assessing a lung nodule of a subject, comprising: (a) assaying a biological sample obtained or derived from the subject to produce a data set comprising gene expression measurements of the biological sample at each of a plurality of lung disease-associated genomic loci, wherein the plurality of disease-associated genomic loci comprises at least one gene selected from the group of genes listed in any one or more of Tables 1, 2, 3, 4, 5, 7, and 8; (b) analyzing the data set to classify the lung nodule of the subject as a malignant lung nodule or a benign lung nodule; and (c) electronically outputting a report indicative of the classification of the lung nodule of the subject as the malignant lung nodule or the benign lung nodule. Gene expression of the biological sample can be measured by, e.g., assaying RNA produced from genomic loci, e.g., lung-disease-associated genes. The gene expression measurement in the biological sample can be performed using any suitable technique, such any suitable RNA quantification techniques, including but not limited to RNA-seq, Ampli-seq, or the like. In some embodiments, the dataset further comprises, clinical characteristics data of one or more clinical characteristics of the subject. In some embodiments, the one or more clinical characteristics are selected from the group of clinical characteristics listed in Table 6.

In some embodiments, the plurality of disease-associated genomic loci comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, or 180 genes selected from the group of genes listed in Table 1.

In some embodiments, the plurality of disease-associated genomic loci comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, or 175 genes selected from the group of genes listed in Table 2.

In some embodiments, the plurality of disease-associated genomic loci comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, or 60 genes selected from the group of genes listed in Table 3.

In some embodiments, the plurality of disease-associated genomic loci comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295 genes selected from the group of genes listed in Table 4.

In some embodiments, the plurality of disease-associated genomic loci comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, or 142 genes selected from the group of genes listed in Table 5.

In some embodiments, the plurality of disease-associated genomic loci comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7. In some embodiments, the genes are selected from BCAT1, CRCP, COA4, OVCA2, POM121, HLA-DPA1, VPS37C, MGST2, RNF220, HDAC3, NFE2L1, WDR20, CNPY4, HOXB2, C6orf120, TMEM8A, ASAP1-IT2, C15orf54, CD101, FNBP1, TECR, PROK2, SLC35B3, TDRD9, CLHC1, LPL, IFITM3, OGFOD3, EIF2B3, TMEM65, and MKRN3. In some embodiments, the plurality of disease-associated genomic loci comprises the genes BCAT1, CRCP, COA4, OVCA2, POM121, HLA-DPA1, VPS37C, MGST2, RNF220, HDAC3, NFE2L1, WDR20, CNPY4, HOXB2, C6orf120, TMEM8A, ASAP1-IT2, C15orf54, CD101, FNBP1, TECR, PROK2, SLC35B3, TDRD9, CLHC1, LPL, IFITM3, OGFOD3, EIF2B3, TMEM65, and MKRN3. In some embodiments, the plurality of disease-associated genomic loci consists of the genes BCAT1, CRCP, COA4, OVCA2, POM121, HLA-DPA1, VPS37C, MGST2, RNF220, HDAC3, NFE2L1, WDR20, CNPY4, HOXB2, C6orf120, TMEM8A, ASAP1-IT2, C15orf54, CD101, FNBP1, TECR, PROK2, SLC35B3, TDRD9, CLHC1, LPL, IFITM3, OGFOD3, EIF2B3, TMEM65, and MKRN3. These genes and those described herein are known to those of skill in the art, and described in the literature. Table A provides examples of Gene ID numbers for genes listed herein in the Tables, including Tables 7 and 8, as described in OMIM®—Online Mendelian Inheritance in Man (McKusick-Nathans Institute of Genetic Medicine, Johns Hopkins University School of Medicine, Baltimore, MD) and in the National Center for Biotechnology Information gene database (NCBI, U.S. National Library of Medicine 8600 Rockville Pike, Bethesda MD, 20894 USA), each incorporated by reference herein in their entirety.

In some embodiments, the plurality of disease-associated genomic loci comprises at least 2, 3, 4, 5, 6, 7, 8, 9, 10, 11, 12, 13, 14, 15, 16, 17, 18, 19, 20, or 21, genes selected from the group of genes listed in Table 8. In some embodiments, the genes are selected from BCAT1, USP32P2, CD177, QPCT, SCAF4, SNRPD3, BCL9L, THBS1, SLC22A18AS, ARCN1, DHX16, SATB1, ST6GAL1, CXCL1, TDRD9, ZNF831, MTCH1, FAM86HP, DHX8, RNF114, and DCTN4. In some embodiments, the plurality of disease-associated genomic loci comprises the genes BCAT1, USP32P2, CD177, QPCT, SCAF4, SNRPD3, BCL9L, THBS1, SLC22A18AS, ARCN1, DHX16, SATB1, ST6GAL1, CXCL1, TDRD9, ZNF831, MTCH1, FAM86HP, DHX8, RNF114, and DCTN4. In some embodiments, the plurality of disease-associated genomic loci consists of the genes BCAT1, USP32P2, CD177, QPCT, SCAF4, SNRPD3, BCL9L, THBS1, SLC22A18AS, ARCN1, DHX16, SATB1, ST6GAL1, CXCL1, TDRD9, ZNF831, MTCH1, FAM86HP, DHX8, RNF114, and DCTN4

In some embodiments, the one or more clinical characteristics includes, 1, 2, 3, 4, 5, 6, 7 or 8 clinical characteristics selected from the group of clinical characteristics listed in Table 6. In some embodiments, the one or more clinical characteristics includes size of the nodule. In some embodiments, the one or more clinical characteristics includes age of the patient. In some embodiments, the one or more clinical characteristics includes presence of the nodule in the lung upper lobe. In some embodiments, the one or more clinical characteristics include size of the nodule, age of the patient, presence of the nodule in the lung upper lobe, or any combination thereof.

In some embodiments, the plurality of disease-associated genomic loci comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7, and the one or more clinical characteristics comprises 1, 2, 3, 4, 5, 6, 7 or 8 clinical characteristics selected from the group of clinical characteristics listed in Table 6. In some embodiments, the plurality of disease-associated genomic loci comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7, and the one or more clinical characteristics of the subject includes size of the nodule, age of the patient, presence of the nodule in the lung upper lobe, or any combination thereof.

In some embodiments, the plurality of disease-associated genomic loci comprise the 31 genes listed in Table 7, and the one or more clinical characteristics comprise the size of the nodule, age of the patient, and the presence of the nodule in the lung upper lobe. In some embodiments, the plurality of disease-associated genomic loci consist of the 31 genes listed in Table 7, and the one or more clinical characteristics consist of the size of the nodule, age of the patient, and the presence of the nodule in the lung upper lobe.

In some embodiments, the method further comprises classifying the lung nodule of the subject as the malignant lung nodule or the benign lung nodule with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises classifying the lung nodule of the subject as the malignant lung nodule or the benign lung nodule with an sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises classifying the lung nodule of the subject as the malignant lung nodule or the benign lung nodule with an specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises classifying the lung nodule of the subject as the malignant lung nodule or the benign lung nodule with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises classifying the lung nodule of the subject as the malignant lung nodule or the benign lung nodule with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises classifying the lung nodule of the subject as the malignant lung nodule or the benign lung nodule with an Area-Under-Curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

In some embodiments, the subject has a lung cancer. In some embodiments, the subject is suspected of having a lung cancer. In some embodiments, the subject is at elevated risk of having a lung cancer. In some embodiments, the subject is asymptomatic for a lung cancer.

In certain embodiments, the method comprises performing biopsy of the lung nodule of the subject based at least in part on the classification of the lung nodule of the subject as the malignant lung nodule or the benign lung nodule. In certain embodiments, the method comprises performing biopsy of the lung nodule of the subject based at least in part on the classification of the lung nodule of the subject as the malignant lung nodule. In some embodiments, the method further comprises administering a treatment to the subject based at least in part on the classification of the lung nodule of the subject as the malignant lung nodule or the benign lung nodule. In some embodiments, the method comprises administering a treatment to the subject based at least in part on the classification of the lung nodule of the subject as the malignant lung nodule. In some embodiments, the method comprises administering a treatment to the subject based at least in part on the classification of the lung nodule of the subject as the malignant lung nodule. In some embodiments, the treatment is configured to treat a lung cancer of the subject. In some embodiments, the treatment is configured to reduce a severity of a lung cancer of the subject. In some embodiments, the treatment is configured to reduce a risk of having a lung cancer of the subject. The treatment can include one or more treatments of lung cancer. In some embodiments, the treatment is selected from the group consisting of: surgery, chemotherapy, targeted therapy, immunotherapy, radiotherapy, and any combination thereof.

In some embodiments, (b) comprises using a trained machine learning classifier to analyze the data set to classify the lung nodule of the subject as the malignant lung nodule or the benign lung nodule. The trained machine-learning model can generate an inference of whether the data set is indicative of a malignant lung nodule or a benign lung nodule. In some embodiments, the machine-learning model, can be trained using gene expression data, and optionally clinical characteristics data. Gene expression data can be obtained by a data analysis tool selected from the group consisting of: a BIG-C™ big data analysis tool, an I-Scope™ big data analysis tool, a T-Scope™ big data analysis tool, a CellScan big data analysis tool, an MS (Molecular Signature) Scoring™ analysis tool, and a Gene Set Variation Analysis (GSVA) tool (e.g., P-Scope).

For example, one or more of a BIG-C™ big data analysis tool, an I-Scope™ big data analysis tool, a T-Scope™ big data analysis tool, a CellScan big data analysis tool, an MS (Molecular Signature) Scoring™ analysis tool, and a Gene Set Variation Analysis (GSVA) tool (e.g., P-Scope) may be used to perform data analysis; which are described by, for example, international application No. PCT/US2019/060641 (filed Nov. 8, 2019, published as WO2020102043A1), which is incorporated by reference herein in its entirety.

In some embodiments, the trained machine learning classifier is a supervised machine learning algorithm or an unsupervised machine learning algorithm. In some embodiments, the trained machine learning classifier is selected from the group consisting of a linear regression, a logistic regression (LOG), a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, a linear discriminant analysis (LDA), a decision tree learning (DTREE), an adaptive boosting (ADB) and any combination thereof. In some embodiments, the trained machine learning classifier comprises the LOG. In some embodiments, the trained machine learning classifier comprises the Ridge regression. In some embodiments, the trained machine learning classifier comprises the Lasso regression. In some embodiments, the trained machine learning classifier comprises the GLM. In some embodiments, the trained machine learning classifier comprises the kNN. In some embodiments, the trained machine learning classifier comprises the SVM. In some embodiments, the trained machine learning classifier comprises the GBM. In some embodiments, the trained machine learning classifier comprises the RF. In some embodiments, the trained machine learning classifier comprises the NB. In some embodiments, the trained machine learning classifier comprises the EN regression. In some embodiments, the trained machine learning classifier comprises the neural network. In some embodiments, the trained machine learning classifier comprises the deep learning algorithm. In some embodiments, the trained machine learning classifier comprises the LDA. In some embodiments, the trained machine learning classifier comprises the DTREE. In some embodiments, the trained machine learning classifier comprises the ADB.

In some embodiments, the method can include receiving, as an output of the machine-learning model, the inference indicating whether the data set is indicative of the malignant lung nodule or the benign lung nodule, and/or electronically outputting a report classifying the lung nodule of the patient as the malignant lung nodule or the benign lung nodule.

In some embodiments, (b) comprises comparing the data set to a reference data set. In some embodiments, the reference data set comprises gene expression measurements of reference biological samples from reference subjects at each of the plurality of lung disease-associated genomic loci, and optionally clinical characteristics data of one or more clinical characteristics selected from the group listed in Table 6. In some embodiments, the reference biological samples comprise a first plurality of biological samples obtained or derived from subjects having a malignant lung nodule and a second plurality of biological samples obtained or derived from subjects having a benign lung nodule.

In some embodiments, the biological sample is selected from the group consisting of: a blood sample, isolated peripheral blood mononuclear cells (PBMCs), a lung biopsy sample, nasal fluid, saliva, and any derivative thereof.

In some embodiments, the method further comprises determining a likelihood of the classification of the lung nodule of the subject as the malignant lung nodule or the benign lung nodule. In some embodiments, the likelihood is about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100%. In some embodiments, the likelihood is least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises monitoring the lung nodule of the subject, wherein the monitoring comprises assessing the lung nodule of the subject at a plurality of time points. In some embodiments, a difference in the assessment of the lung nodule of the subject among the plurality of time points is indicative of one or more clinical indications selected from the group consisting of: (i) a diagnosis of the lung nodule of the subject, (ii) a prognosis of the lung nodule of the subject, and (iii) an efficacy or non-efficacy of a course of treatment for treating the lung nodule of the subject. In some embodiments, the plurality of time points comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 different time points.

In an aspect, the present disclosure provides a method for assessing a lung nodule of a patient. The method can include, any one of, any combination of, or all of steps a′, b′, c′ and d′. Step a′ can include obtaining a data set containing gene expression measurements of a biological sample obtained or derived from the patient, of at least two lung disease-associated genes. The data set can be obtained by assaying the biological sample. In some embodiments, the at least two lung disease-associated genes are selected from the group of genes listed in Table 4. In some embodiments, the at least two lung disease-associated genes are selected from the group of genes listed in Table 1. In some embodiments, the at least two lung disease-associated genes are selected from the group of genes listed in Table 2. In some embodiments, the at least two lung disease-associated genes are selected from the group of genes listed in Table 3. In some embodiments, the at least two lung disease-associated genes are selected from the group of genes listed in Table 5. In some embodiments, the at least two lung disease-associated genes are selected from the group of genes listed in Table 7. In some embodiments, the at least two lung disease-associated genes are selected from the group of genes listed in Table 8. Step b′ can include providing the data set as an input to a machine-learning model trained to generate an inference of whether the data set is indicative of a malignant lung nodule or a benign lung nodule. Step c′ can include receiving, as an output of the machine-learning model, the inference indicating whether the data set is indicative of the malignant lung nodule or the benign lung nodule. Step d′ can include electronically outputting a report classifying the lung nodule of the patient as the malignant lung nodule or the benign lung nodule. In some embodiments, the data set of step a′, can further include clinical characteristics data of one or more clinical characteristics of the patient. In some embodiments, the one or more clinical characteristics are selected from the group of clinical characteristics listed in Table 6. In some embodiments, the data set of step a′, contains i) gene expression measurements of the biological sample of at least two lung disease-associated genes selected from the group of genes listed in any one or more of Tables 1, 2, 3, 4, 5, 7, and 8, and ii) optionally clinical characteristics data of one or more clinical characteristics of the patient selected from the group of clinical characteristics listed in Table 6. The gene expression measurement of the biological sample can be performed using any suitable technique, such as any suitable RNA quantification technique, including but not limited to RNA-seq, Ampli-seq, and the like.

In some embodiments, the at least two lung disease-associated genes, e.g. as of step a′, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, or 182, or any value or range there between, genes selected from the group of genes listed in Table 1. In some embodiments, the at least two lung disease-associated genes, e.g. as of step a′, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, or 175, or any value or range there between, genes selected from the group of genes listed in Table 2. In some embodiments, the at least two lung disease-associated genes, e.g. as of step a′, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 or 62, or any value or range there between, genes selected from the group of genes listed in Table 3. In some embodiments, the at least two lung disease-associated genes, e.g. as of step a′, include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295, or any value or range there between, genes selected from the group of genes listed in Table 4. In some embodiments, the at least two lung disease-associated genes, e.g. as of step a′, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, or 142, or any value or range there between, genes selected from the group of genes listed in Table 5. In some embodiments, the at least two lung disease-associated genes, e.g. as of step a′, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7. In some embodiments, the at least two lung disease-associated genes of step a′, are selected from BCAT1, CRCP, COA4, OVCA2, POM121, HLA-DPA1, VPS37C, MGST2, RNF220, HDAC3, NFE2L1, MKRN3. In some embodiments, the at least two lung disease-associated genes, e.g. as of step a′, include at least 2, 3, 4, 5, 6, 7, 8, 9, 10, 11, 12, 13, 14, 15, 16, 17, 18, 19, 20, or 21 genes selected from the group of genes listed in Table 8. In some embodiments, the at least two lung disease-associated genes of step a′, are selected from BCAT1, USP32P2, CD177, QPCT, SCAF4, SNRPD3, BCL9L, THBS1, SLC22A18AS, ARCN1, DHX16, SATB1, ST6GAL1, CXCL1, TDRD9, ZNF831, MTCH1, FAM86HP, DHX8, RNF114, and DCTN4. In some embodiments, the one or more clinical characteristics includes, 1, 2, 3, 4, 5, 6, 7 or 8 clinical characteristics selected from the group of clinical characteristics listed in Table 6. In some embodiments, the one or more clinical characteristics includes size of the nodule. In some embodiments, the one or more clinical characteristics includes age of the patient. In some embodiments, the one or more clinical characteristics includes presence of the nodule in the lung upper lobe. In some embodiments, the one or more clinical characteristics of the patient includes size of the nodule, age of the patient, presence of the nodule in the lung upper lobe, or any combination thereof. In some embodiments, the at least two lung disease-associated genes of step a′, comprise the 31 genes listed in Table 7, and the one or more clinical characteristics of step a′, comprise the size of the nodule, age of the patient, and the presence of the nodule in the lung upper lobe. In some embodiments, the at least two lung disease-associated genes of step a′, consist of the 31 genes listed in Table 7, and the one or more clinical characteristics of step a′, consist of the size of the nodule, age of the patient, and the presence of the nodule in the lung upper lobe. In some embodiments, the data set of step a′, contains i) gene expression measurements of the biological sample of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease-associated genes selected from the group of genes listed in Table 7, and ii) clinical characteristics data of 1, 2, 3, 4, 5, 6, 7 or 8 clinical characteristics of the patient selected from the group of clinical characteristics listed in Table 6. In some embodiments, the data set of step a′, contains i) gene expression measurements of the biological sample of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease-associated genes selected from the group of genes listed in Table 7, and ii) clinical characteristics data of the clinical characteristics of the patient selected from size of the nodule, age of the patient, presence of the nodule in the lung upper lobe, or any combination thereof.

In some embodiments, the biological sample is a blood sample, isolated peripheral blood mononuclear cells (PBMCs), a lung biopsy sample, nasal fluid, saliva, or any derivative thereof. In some embodiments, the biological sample is a blood sample or any derivative thereof. In some embodiments, the biological sample is isolated peripheral blood mononuclear cells (PBMCs) or any derivative thereof. In some embodiments, the biological sample is a lung biopsy sample, or any derivative thereof. In some embodiments, the biological sample is a nasal fluid sample, or any derivative thereof. In some embodiments, the biological sample is a saliva sample, or any derivative thereof.

The method can classify the lung nodule of the patient as the malignant lung nodule or the benign lung nodule with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model, e.g. of step b′, can infer whether the data set is indicative of a malignant lung nodule or a benign lung nodule with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

The method can classify the lung nodule of the patient as the malignant lung nodule or the benign lung nodule with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model, e.g. of step b′, can infer whether the data set is indicative of a malignant lung nodule or a benign lung nodule with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

The method can classify the lung nodule of the patient as the malignant lung nodule or the benign lung nodule with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model, e.g. of step b′, can infer whether the data set is indicative of a malignant lung nodule or a benign lung nodule with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

The method can classify the lung nodule of the patient as the malignant lung nodule or the benign lung nodule with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model, e.g. of step b′, can infer whether the data set is indicative of a malignant lung nodule or a benign lung nodule with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

The method can classify the lung nodule of the patient as the malignant lung nodule or the benign lung nodule with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model, e.g. of step b′, can infer whether the data set is indicative of a malignant lung nodule or a benign lung nodule with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

The machine learning model, e.g. of step b′, can infer whether the data set is indicative of a malignant lung nodule or a benign lung nodule with a receiver operating characteristic (ROC) curve with an AUC of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

The inference from the machine learning model can include a confidence value between 0 and 1, such as, 0, 0.1, 0.2, 0.3, 0.4, 0.5, 0.6, 0.7, 0.8, 0.9 or 1, or any value or range there between, that the nodule is malignant. Higher confidence values may be correlated with a higher likelihood that the nodule is malignant. A malignant nodule may be characterized by the ability to metastasize or grow invasively, which may be in contrast to benign nodules.

In some embodiments, the patient has a lung cancer. In some embodiments, the patient does not have lung cancer. In some embodiments, the patient is suspected of having a lung cancer. In some embodiments, the patient is at an elevated risk of having a lung cancer. In some embodiments, the patient is asymptomatic for a lung cancer.

In certain embodiments, the method includes optionally performing biopsy of the lung nodule of the patient based at least in part on the classification of the lung nodule of the patient as the malignant lung nodule or the benign lung nodule. In certain embodiments, the method includes optionally performing a biopsy of the lung nodule of the patient based at least in part on the classification of the lung nodule of the patient as the malignant lung nodule. In some embodiments, a biopsy is performed. In some embodiments, a biopsy is not performed. The decision to perform a biopsy may be made by one of skill in the art, based on knowledge and experience, in view of the classification of the lung nodule of the patient as the malignant lung nodule or the benign lung nodule. The decision to perform a biopsy may depend in part on the confidence value of the inference. In some embodiments, the method further comprises administering a treatment to the patient based at least in part on the classification of the lung nodule of the patient as the malignant lung nodule or the benign lung nodule. In some embodiments, the method comprises administering a treatment to the patient based at least in part on the classification of the lung nodule of the patient as the malignant lung nodule. In some embodiments, the treatment is configured to treat a lung cancer of the patient. In some embodiments, the treatment is configured to reduce a severity of a lung cancer of the patient. In some embodiments, the treatment is configured to reduce a risk of having a lung cancer of the patient. The treatment can include one or more treatments of lung cancer. In some embodiments, the treatment is surgery, chemotherapy, targeted therapy, immunotherapy, radiotherapy, or any combination thereof.

The trained machine-learning model, e.g. of step b′, can generate the inference of whether the data set is indicative of a malignant lung nodule or a benign lung nodule, by comparing the data set to a reference data set. The machine-learning model can be trained using the reference data set. In some embodiments, the reference data set contains gene expression measurements of a plurality of genes of a plurality of reference biological samples from a plurality of reference subjects having lung nodule; data regarding whether the lung nodules of the reference subjects are benign or malignant; and optionally clinical characteristics data of the reference subjects of one or more clinical characteristics selected from the group of clinical characteristics listed in Table 6. A first portion of the plurality of reference subjects can have benign lung nodule, and a second portion of the plurality of reference subjects can have malignant lung nodule. In some embodiments, the reference data set contains a plurality of individual reference data sets. A respective individual reference data set of the plurality of individual reference data sets can contain i) gene expression measurements of a plurality of genes of a reference biological sample from a reference subject having a lung nodule, ii) data regarding whether the lung nodule of the reference subject is benign or malignant, and iii) optionally clinical characteristics data of one or more clinical characteristics selected from the group of clinical characteristics listed in Table 6, of the reference subject. The plurality of individual reference data sets can be obtained from the plurality of reference subjects. In some embodiments, different individual reference data sets are obtained from different reference subjects. In some embodiments, each of the individual reference data set contains i) gene expression measurements of the plurality of genes of a reference biological sample from one reference subject, ii) data regarding whether the lung nodule of the one reference subject is benign or malignant, and iii) optionally clinical characteristics data of the one or more clinical characteristics selected from the group of clinical characteristics listed in Table 6 of the one reference subject, wherein different individual reference data sets are obtained from different reference subjects. In certain embodiments, oversampling or undersampling correction is made during training of the machine learning model. For example, if a reference data set includes a greater number of samples identified as benign and a relatively fewer number of samples identified as malignant, the malignant samples may be oversampled to produce a data set that has equal number of benign and malignant samples. The plurality of genes of the reference data set can include at least 2 genes selected from the group of genes listed in any one or more of Tables 1, 2, 3, 4, 5, 7, and 8. In some embodiments, the plurality of genes of the reference data set include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, or 182, or any value or range there between, genes selected from the group of genes listed in Table 1. In some embodiments, the plurality of genes of the reference data set include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, or 175, or any value or range there between, genes selected from the group of genes listed in Table 2. In some embodiments, the plurality of genes of the reference data set include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 or 62, or any value or range there between, genes selected from the group of genes listed in Table 3. In some embodiments, the plurality of genes of the reference data set include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295, or any value or range there between, genes selected from the group of genes listed in Table 4. In some embodiments, the plurality of genes of the reference data set include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, or 142, or any value or range there between, genes selected from the group of genes listed in Table 5. In some embodiments, the plurality of genes of the reference data set include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7. In some embodiments, the plurality of genes of the reference data set include at least 2, 3, 4, 5, 6, 7, 8, 9, 10, 11, 12, 13, 14, 15, 16, 17, 18, 19, 20, or 21, genes selected from the group of genes listed in Table 8. In some embodiments, the one or more clinical characteristics of the reference data set include, 1, 2, 3, 4, 5, 6, 7 or 8 clinical characteristics selected from the group of clinical characteristics listed in Table 6. In some embodiments, the one or more clinical characteristics of the reference data set includes size of the nodule. In some embodiments, the one or more clinical characteristics of the reference data set includes age of the patient. In some embodiments, the one or more clinical characteristics of the reference data set includes presence of the nodule in the lung upper lobe. In some embodiments, the one or more clinical characteristics of the reference data set includes size of the nodule, age of the patient, presence of the nodule in the lung upper lobe, or any combination thereof. In some embodiments, the plurality of genes of the reference data set include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7, and the one or more clinical characteristics of the reference data set includes 1, 2, 3, 4, 5, 6, 7 or 8 clinical characteristics selected from the group of clinical characteristics listed in Table 6. In some embodiments, the plurality of genes of the reference data set include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7, and the one or more clinical characteristics of the reference data set include size of the nodule, age of the patient, presence of the nodule in the lung upper lobe, or any combination thereof. In some embodiments, the plurality of genes of the reference data set include comprise the 31 genes listed in Table 7, and the one or more clinical characteristics of the reference data set comprise the size of the nodule, age of the patient, and the presence of the nodule in the lung upper lobe. In some embodiments, the plurality of genes of the reference data set consist of the 31 genes listed in Table 7, and the one or more clinical characteristics of the reference data set consist of the size of the nodule, age of the patient, and the presence of the nodule in the lung upper lobe.

The genes of the data set and genes of the reference data set can at least partially overlap and/or the optional clinical characteristics of the data set and optional clinical characteristics of the reference data set can at least partially overlap. In some embodiments, the reference biological sample is a blood sample, isolated peripheral blood mononuclear cells (PBMCs), a lung biopsy sample, nasal fluid, saliva, or any derivative thereof. In some embodiments, the reference biological sample is a blood sample or any derivative thereof. In some embodiments, the reference biological sample is isolated peripheral blood mononuclear cells (PBMCs) or any derivative thereof. In some embodiments, the reference biological sample is a lung biopsy sample, or any derivative thereof. In some embodiments, the reference biological sample is a nasal fluid sample, or any derivative thereof. In some embodiments, the reference biological sample is a saliva sample, or any derivative thereof. The reference subjects can be human.

Gene expression data can be obtained by a data analysis tool selected from the group: a BIG-C™ big data analysis tool, an I-Scope™ big data analysis tool, a T-Scope™ big data analysis tool, a Cell Scan big data analysis tool, an MS (Molecular Signature) Scoring™ analysis tool, and a Gene Set Variation Analysis (GSVA) tool (e.g., P-Scope).

In some embodiments, the trained machine learning model, e.g. of step b′, is a supervised machine learning algorithm or an unsupervised machine learning algorithm. In some embodiments, the trained machine learning model is trained using linear regression, logistic regression (LOG), Ridge regression, Lasso regression, elastic net (EN) regression, support vector machine (SVM), gradient boosted machine (GBM), k nearest neighbors (kNN), generalized linear model (GLM), naïve Bayes (NB), neural network, Random Forest (RF), deep learning algorithm, linear discriminant analysis (LDA), a decision tree learning (DTREE), adaptive boosting (ADB), or any combination thereof. In some embodiments, the trained machine learning model is trained using LOG. In some embodiments, the trained machine learning model is trained using Ridge regression. In some embodiments, the trained machine learning model is trained using Lasso regression. In some embodiments, the trained machine learning model is trained using GLM. In some embodiments, the trained machine learning model is trained using kNN. In some embodiments, the trained machine learning model is trained using SVM. In some embodiments, the trained machine learning model is trained using GBM. In some embodiments, the trained machine learning model is trained using RF. In some embodiments, the trained machine learning model is trained using NB. In some embodiments, the trained machine learning model is trained using the EN regression. In some embodiments, the trained machine learning model is trained using neural network. In some embodiments, the trained machine learning model is trained using deep learning algorithm. In some embodiments, the trained machine learning model is trained using LDA. In some embodiments, the trained machine learning model is trained using DTREE. In some embodiments, the trained machine learning model is trained using ADB.

In some embodiments, the method comprises determining a likelihood of the classification of the lung nodule of the patient as the malignant lung nodule or the benign lung nodule. In some embodiments, the likelihood is about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or about 100%. In some embodiments, the likelihood is least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In some embodiments, the method further comprises monitoring the lung nodule of the patient, wherein the monitoring comprises assessing the lung nodule of the patient at a plurality of time points. In some embodiments, a difference in the assessment of the lung nodule of the patient among the plurality of time points is indicative of one or more clinical indications selected from the group consisting of: (i) a diagnosis of the lung nodule of the patient, (ii) a prognosis of the lung nodule of the patient, and (iii) an efficacy or non-efficacy of a course of treatment for treating the lung nodule of the patient. In some embodiments, the plurality of time points comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 different time points.

In another aspect, the present disclosure provides a method for determining a gene set capable of classifying a lung nodule, benign or malignant. Gene expression measurements of one or more genes of the gene set, of a biological sample (e.g. blood) from a subject can be used to classify a lung nodule of the subject, benign or malignant without performing biopsy of the nodule. In some embodiments, a biopsy of the nodule is performed to confirm and/or follow-up the classification results obtained by using the gene expression measurements data. In some embodiments, a biopsy of the nodule is not performed. The method can include any one of, any combination of, or all of steps a″, b″, c″ and d″. In step a″, a reference data set can be obtained and/or provided. The reference data set can contain a plurality of individual reference data sets. A respective individual reference data set of the plurality of individual reference data sets can contain i) gene expression measurements of a plurality of genes of a reference biological sample from a reference subject having a lung nodule, ii) data regarding whether the lung nodule is benign or malignant, and iii) optionally clinical characteristics data of one or more clinical characteristics selected from the group of clinical characteristics listed in Table 6 of the reference subject. The plurality of individual reference data sets can be obtained from a plurality of reference subjects. In some embodiments, different individual reference data sets are obtained from different reference subjects. In some embodiments, each of the individual reference data set contains i) gene expression measurements of the plurality of genes of a reference biological sample from one reference subject, ii) data regarding whether the lung nodule of the one reference subject is benign or malignant, and iii) optionally clinical characteristics data of the one or more clinical characteristics selected from the group of the clinical characteristics listed in Table 6 of the one reference subject, wherein different individual reference data sets are obtained from different reference subjects. A first portion of the plurality of reference subjects can have benign lung nodule, and a second portion of the plurality of reference subjects can have malignant lung nodule. The reference data set can contain gene expression measurements of the plurality of genes of a plurality of reference biological samples from the plurality of reference subjects having lung nodule; data regarding whether the lung nodules of the reference subjects are benign or malignant; and optionally clinical characteristics data of the reference subjects of one or more clinical characteristics selected from the group of clinical characteristics listed in Table 6. In step b″, a machine learning model can be trained using the reference data set to infer whether a lung nodule is benign or malignant based on at least in part on one or more predictors selected from i) the plurality of genes, and ii) optionally the one or more clinical characteristics. The trained machine learning model can infer whether the lung nodule from a subject is benign or malignant based on at least in part on the gene expression measurements of the plurality of genes from a biological sample of the subject, and optionally clinical characteristics data of the one or more clinical characteristics of the subject. In some embodiments, the machine learning model can be trained using a training data set containing a first portion of the reference data set, and a validation data set containing a second portion of the reference data set. In certain embodiments, oversampling or undersampling correction is made during training of the machine learning model. For example, if a data set includes a greater number of samples identified as benign and a relatively fewer number of samples identified as malignant, the malignant samples may be oversampled to produce a data set that has equal number of benign and malignant samples. In step c″, feature importance values of the plurality of genes can be determined. In step d″, the gene set can be selected. In some embodiments, the gene set is selected as predictors that are used to train the machine learning model. The gene set, may be selected based at least in part on the feature importance values. In some embodiments, the feature importance values of the genes of the gene set, are within top 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, or 250, or any value or range there between, feature importance values of the plurality of genes. In some embodiments, the feature importance of the genes of the gene set, have accuracy, greater than 30%, 35%, 40%, 45%, 50%, 55%, 60%, 65%, 70%, 75%, 80% or 90%. In some embodiments, the feature importance of the genes of the gene set, have threshold importance, greater than 30, 35, 40, 45, 50, 55, 60, 65, 70, 75, 80 or 90. In certain embodiments, the top 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, or 250, or any value or range there between, predictors of the machine learning model include the genes of the gene set. In some embodiments, the plurality of genes of the reference data set of step a″, include at least 2 genes selected from the group of genes listed in Table 9. In some embodiments, the plurality of genes of the reference data set of step a″, include at least 2 genes selected from the group of genes listed in Table 1. In some embodiments, the plurality of genes of the reference data set of step a″, include at least 2 genes selected from the group of genes listed in Table 2. In some embodiments, the plurality of genes of the reference data set of step a″, include at least 2 genes selected from the group of genes listed in Table 3. In some embodiments, the plurality of genes of the reference data set of step a″, include at least 2 genes selected from the group of genes listed in Table 4. In some embodiments, the plurality of genes of the reference data set of step a″, include at least 2 genes selected from the group of genes listed in Table 5. In some embodiments, the plurality of genes of the reference data set of step a″, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 300, 400, 500, 600, 700, 800, 900, 1000, 1100 or 1178, or any value or range there between, genes selected from the group of genes listed in Table 9. In some embodiments, the plurality of genes of the reference data set of step a″, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, or 182, or any value or range there between, genes selected from the group of genes listed in Table 1. In some embodiments, the plurality of genes of the reference data set of step a″, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, or 175, or any value or range there between, genes selected from the group of genes listed in Table 2. In some embodiments, the plurality of genes of the reference data set of step a″, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 or 62, or any value or range there between, genes selected from the group of genes listed in Table 3. In some embodiments, the plurality of genes of the reference data set of step a″, include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295, or any value or range there between, genes selected from the group of genes listed in Table 4. In some embodiments, the plurality of genes of the reference data set of step a″, include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, or 142, or any value or range there between, genes selected from the group of genes listed in Table 5. In some embodiments, the one or more clinical characteristics of the reference data set of step a″, include 1, 2, 3, 4, 5, 6, 7, or 8, clinical characteristics selected from the group of clinical characteristics listed in Table 6. In some embodiments, the plurality of genes of the reference data set of step a″, include at least 2 genes selected from a group of genes listed in any one or more of Tables 1, 2, 3, 4, 5 or 9 or any combination thereof, and the one or more clinical characteristics of the reference data set of step a″, include 1, 2, 3, 4, 5, 6, 7, or 8 clinical characteristics selected from the group of clinical characteristics listed in Table 6. In some embodiments, genes having collinear expression with correlation coefficients (e.g. in non-limiting aspects >0.7 to >0.9) were removed from the reference data set. Collinear gene expression can be measured by any suitable technique, e.g. Pearson correlation coefficient. While determining feature importance values for the plurality of genes has been described, this is merely a non-limiting illustrative example of a techniques that can be practiced. In various embodiments, one or more feature selection techniques are used to determine the gene set that can classify a lung nodule benign or malignant. Feature selection techniques can include least absolute shrinkage and selection operator (Lasso) regression, support vector machine (SVM), regularized trees, decision trees, memetic algorithm, random multinomial logit (RMNL), auto-encoding networks, submodular feature selection, recursive feature elimination, or any combination thereof. In some of these cases, feature importance values need not be calculated for each of the genes in Table 9. The reference biological sample can be a blood sample, isolated peripheral blood mononuclear cells (PBMCs), lung biopsy sample, nasal fluid sample, saliva sample, or any derivative thereof. In some embodiments, the reference biological sample is a blood sample or any derivative thereof. In some embodiments, the reference biological sample is isolated peripheral blood mononuclear cells (PBMCs) or any derivative thereof. In some embodiments, the reference biological sample is a lung biopsy sample, or any derivative thereof. In some embodiments, the reference biological sample is a nasal fluid sample, or any derivative thereof. In some embodiments, the reference biological sample is a saliva sample, or any derivative thereof.

The machine learning model, e.g. of step b″, can be trained using a supervised machine learning algorithm or an unsupervised machine learning algorithm. In some embodiments, the machine learning model, e.g. of step b″, is trained using linear regression, logistic regression, Ridge regression, Lasso regression, elastic net (EN) regression, support vector machine (SVM), gradient boosted machine (GBM), k nearest neighbors (kNN), generalized linear model (GLM), naïve Bayes (NB), neural network, Random Forest (RF), deep learning algorithm, linear discriminant analysis (LDA), a decision tree learning (DTREE), adaptive boosting (ADB), or any combination thereof. In some embodiments, the machine learning model is trained using logistic regression. In some embodiments, the machine learning model is trained using Ridge regression. In some embodiments, the machine learning model is trained using Lasso regression. In some embodiments, the machine learning model is trained using GLM. In some embodiments, the machine learning model is trained using kNN. In some embodiments, the machine learning model is trained using SVM. In some embodiments, the machine learning model is trained using GBM. In some embodiments, the machine learning model is trained using RF. In some embodiments, the machine learning model is trained using NB. In some embodiments, the machine learning model is trained using the EN regression. In some embodiments, the machine learning model is trained using neural network. In some embodiments, the machine learning model is trained using deep learning algorithm. In some embodiments, the machine learning model is trained using LDA. In some embodiments, the machine learning model is trained using DTREE. In some embodiments, the machine learning model is trained using ADB.

The gene set can classify a lung nodule as the malignant lung nodule or the benign lung nodule with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The gene set can classify a lung nodule as the malignant lung nodule or the benign lung nodule with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The gene set can classify a lung nodule as the malignant lung nodule or the benign lung nodule with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The gene set can classify a lung nodule as the malignant lung nodule or the benign lung nodule with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The gene set can classify a lung nodule as the malignant lung nodule or the benign lung nodule with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

In another aspect, the present disclosure provides a method for developing a trained machine learning model capable of inferring whether a lung nodule of a patient is benign or malignant. The method can include any one of, any combination of, or all of steps a′″, b″′, c′″, d′″ and e′″. Step a′″, can include obtaining and/or providing a first reference data set. The first reference data set can contain a plurality of first individual reference data sets. A respective first individual reference data set of the plurality of first individual reference data sets can contain i) gene expression measurements of a plurality of genes of a reference biological sample from a reference subject having a lung nodule, ii) data regarding whether the lung nodule of the reference subject is benign or malignant, and iii) optionally clinical characteristics data of one or more clinical characteristics selected from the group of clinical characteristics listed in Table 6 of the reference subject. The plurality of first individual reference data sets can be obtained from a plurality of reference subjects. In some embodiments, different first individual reference data sets are obtained from different reference subjects. In some embodiments, each of the first individual reference data set contains i) gene expression measurements of the plurality of genes of a reference biological sample from one reference subject, iii) data regarding whether the lung nodule of the one reference subject is benign or malignant and iii) optionally clinical characteristics data of the one or more clinical characteristics selected from the group of clinical characteristics listed in Table 6 of the one reference subject, wherein different first individual reference data sets are obtained from different reference subjects. A first portion of the plurality of reference subjects can have benign lung nodule, and a second portion of the plurality of reference subjects can have malignant lung nodule. The first reference data set can contain gene expression measurements of the plurality of genes of a plurality of reference biological samples from the plurality of reference subjects having lung nodule; data regarding whether the lung nodules of the reference subjects are benign or malignant; and optionally clinical characteristics data of the reference subjects of one or more clinical characteristics selected from the group of clinical characteristics listed in Table 6. In step b″′, a first machine learning model can be trained using the first reference data set to infer whether a lung nodule is benign or malignant, based at least in part on one or more predictors selected from i) the plurality of genes, and ii) optionally the one or more clinical characteristics. The first machine learning model can be trained to infer whether the lung nodule from a subject is benign or malignant, based at least in part on i) the gene expression measurement data of the plurality of genes of a biological sample from the subject, and ii) optionally the clinical characteristics data of the one or more clinical characteristics of the subject. In some embodiments, the first machine learning model is trained using a training data set containing a first portion of the first reference data set, and a validation data set containing a second portion of the first reference data set. In step c′″, feature importance values of one or more predictors of the first machine learning model can be determined. In step d′″, A predictors of the first machine learning model based at least in part on the feature importance values can be selected, where A can be an integer from 3 to 2000, such as 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 300, 400, 500, 600, 700, 800, 900, 1000, 1100, 1200, 1300, 1400, 1500, 1600, 1700, 1800, 1900, or 2000, or any integer value or ranges therein. In certain embodiments, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, or 250, or any value or range there between, predictors of the first machine learning model are selected. In some embodiments, the A predictors have top A feature importance values, for example, in a non-limiting aspect, A is 10, and 10 predictors having 10 highest feature importance values are selected. In some embodiments, the feature importance of the A predictors, have an accuracy, greater than 30%, 35%, 40%, 45%, 50%, 55%, 60%, 65%, 70%, 75%, 80% or 90%. In some embodiments, the feature importance of the A predictors, can have a threshold importance, greater than 30, 35, 40, 45, 50, 55, 60, 65, 70, 75, 80 or 90. A predictors can include one or more genes, and/or optionally one or more clinical characteristics. While determining feature importance values for the one or more predictors has been described, this is merely a non-limiting illustrative example of a technique that can be practiced. In various embodiments, one or more feature selection techniques are used to determine the A predictors. Feature selection techniques can include least absolute shrinkage and selection operator (Lasso) regression, support vector machine (SVM), regularized trees, decision trees, memetic algorithm, random multinomial logit (RMNL), auto-encoding networks, submodular feature selection, recursive feature elimination, or any combination thereof. In some of these cases, in step c″′, feature importance values need not be calculated for each of the predictors of first machine learning model. Step e′″, can include training a second machine learning model based at least in part on a second reference data set to obtain the trained machine learning model. The trained machine learning model can infer whether a lung nodule of a subject is benign or malignant, based at least in part on measurement data of the A predictors of the subject. The second reference data set can contain a plurality of second individual reference data sets. A respective second individual reference data set of the plurality of second individual reference data sets can include i) measurement data of the A predictors of a reference subject, and ii) data regarding whether the lung nodule of the reference subject is benign or malignant. Measurement data of the A predictors can include, gene expression measurements of the reference biological sample of the one or more genes predictors of the A predictors, and/or optionally clinical characteristics data of optional one or more clinical characteristics predictors of the A predictors. The plurality of second individual reference data sets can be obtained from the plurality of reference subjects. In some embodiments, different second individual reference data sets are obtained from different reference subjects. In some embodiments, each of the second individual reference data set contains i) measurement data of the A predictors of one reference subject, and ii) data regarding whether the lung nodule of the one reference subject is benign or malignant, wherein different second individual reference data sets are obtained from different reference subjects. In certain embodiments, oversampling or undersampling correction are made during training of the first and/or second machine learning model. The second reference data set can contain measurement data of the A predictors from the plurality of reference subjects, and data regarding whether the lung nodules of the reference subjects are benign or malignant. In some embodiments, the plurality of genes of the first reference data set include at least 2 genes selected from the group of genes listed in Table 9. In some embodiments, the plurality of genes of the first reference data set include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 300, 400, 500, 600, 700, 800, 900, 1000, 1100 or 1178, or any value or range there between, genes selected from the group of genes listed in Table 9. In some embodiments, the plurality of genes of the first reference data set include at least 2 genes selected from the group of genes listed in Table 1. In some embodiments, the plurality of genes of the first reference data set include at least 2 genes selected from the group of genes listed in Table 2. In some embodiments, the plurality of genes of the first reference data set include at least 2 genes selected from the group of genes listed in Table 3. In some embodiments, the plurality of genes of the first reference data set include at least 2 genes selected from the group of genes listed in Table 4. In some embodiments, the plurality of genes of the first reference data set include at least 2 genes selected from the group of genes listed in Table 5. In some embodiments, the one or more clinical characteristics of the first reference data set include 1, 2, 3, 4, 5, 6, 7, or 8 clinical characteristics selected from the group of clinical characteristics listed in Table 6. In some embodiments, the plurality of genes of the first reference data set include at least 2 genes selected from a group of genes listed in any one or more of Tables 1, 2, 3, 4, 5 or 9, and the one or more clinical characteristics of the first reference data set include 1, 2, 3, 4, 5, 6, 7, or 8 clinical characteristics selected from the group of clinical characteristics listed in Table 6. In some embodiments, genes having collinear expression with correlation coefficients (e.g. in non-limiting aspects >0.7 to >0.9) were removed from the reference data set. Collinear gene expression can be measured by any suitable technique, e.g. Pearson correlation coefficient. In some embodiments, the A predictors include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, or 182, or any value or range there between, genes selected from the group of genes listed in Table 1, as predictors. In some embodiments, the A predictors include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, or 175, or any value or range there between, genes selected from the group of genes listed in Table 2, as predictors. In some embodiments, the A predictors include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 or 62, or any value or range there between, genes selected from the group of genes listed in Table 3, as predictors. In some embodiments, the A predictors include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295, or any value or range there between, genes selected from the group of genes listed in Table 4, as predictors. In some embodiments, the A predictors include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, or 142, or any value or range there between, genes selected from the group of genes listed in Table 5, as predictors. In some embodiments, the A predictors include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes of genes selected from the group listed in Table 7, as predictors. In some embodiments, the A predictors can at least 2, 3, 4, 5, 6, 7, 8, 9, 10, 11, 12, 13, 14, 15, 16, 17, 18, 19, 20, or 21 genes selected from the group of genes listed in Table 8, as predictors. In some embodiments, the A predictors include i) at least, 2 genes selected from the group of genes listed in any one or more of Tables 1, 2, 3, 4, 5, 7, and 8, and ii) optionally size of the nodule, age of the patient, presence of the nodule in the lung upper lobe, or any combination thereof, as predictors. In some embodiments, the A predictors can include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33 or 34 predictors selected from the group listed in Table 7. In some embodiments, the A predictors comprise the 34 predictors listed in Table 7. In some embodiments, the A predictors consist the 34 predictors listed in Table 7.

In some embodiments, the reference biological sample is a blood sample, isolated peripheral blood mononuclear cells (PBMCs), a lung biopsy sample, nasal fluid, saliva, or any derivative thereof. In some embodiments, the reference biological sample is a blood sample or any derivative thereof. In some embodiments, the reference biological sample is isolated peripheral blood mononuclear cells (PBMCs) or any derivative thereof. In some embodiments, the reference biological sample is a lung biopsy sample, or any derivative thereof. In some embodiments, the reference biological sample is a nasal fluid sample, or any derivative thereof. In some embodiments, the reference biological sample is a saliva sample, or any derivative thereof. The trained machine learning model, e.g. obtained in step e′″, can infer whether a lung nodule is benign or malignant with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The trained machine learning model, e.g. obtained in step e′″, can infer whether a lung nodule is benign or malignant with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The trained machine learning model, e.g. obtained in step e′″, can infer whether a lung nodule is benign or malignant with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The trained machine learning model, e.g. obtained in step e′″, can infer whether a lung nodule is benign or malignant with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The trained machine learning model, e.g. obtained in step e′″, can infer whether a lung nodule is benign or malignant with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The trained machine learning model, e.g. obtained in step e′″, can infer whether a lung nodule is benign or malignant with a receiver operating characteristic (ROC) curve with an Area-Under-Curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

Gene expression data can be obtained by a data analysis tool selected from the group: a BIG-C™ big data analysis tool, an I-Scope™ big data analysis tool, a T-Scope™ big data analysis tool, a Cell Scan big data analysis tool, an MS (Molecular Signature) Scoring™ analysis tool, and a Gene Set Variation Analysis (GSVA) tool (e.g., P-Scope).

In some embodiments, the trained machine learning model is a supervised machine learning algorithm or an unsupervised machine learning algorithm. In some embodiments, first and/or second machine-learning model is independently trained using linear regression, logistic regression (LOG), Ridge regression, Lasso regression, elastic net (EN) regression, support vector machine (SVM), gradient boosted machine (GBM), k nearest neighbors (kNN), generalized linear model (GLM), naïve Bayes (NB), neural network, Random Forest (RF), deep learning algorithm, linear discriminant analysis (LDA), a decision tree learning (DTREE), adaptive boosting (ADB), or any combination thereof. In some embodiments, the first and/or second machine-learning model is independently trained using LOG. In some embodiments, the first and/or second machine-learning model is independently trained using Ridge regression. In some embodiments, the first and/or second machine-learning model is independently trained using Lasso regression. In some embodiments, the first and/or second machine-learning model is independently trained using GLM. In some embodiments, the first and/or second machine-learning model is independently trained using kNN. In some embodiments, the first and/or second machine-learning model is independently trained using SVM. In some embodiments, the first and/or second machine-learning model is independently trained using GBM. In some embodiments, the first and/or second machine-learning model is independently trained using RF. In some embodiments, the first and/or second machine-learning model is independently trained using NB. In some embodiments, the first and/or second machine-learning model is independently trained using the EN regression. In some embodiments, the first and/or second machine-learning model is independently trained using neural network. In some embodiments, the first and/or second machine-learning model is independently trained using deep learning algorithm. In some embodiments, the first and/or second machine-learning model is independently trained using LDA. In some embodiments, the first and/or second machine-learning model is independently trained using DTREE. In some embodiments, the first and/or second machine-learning model is independently trained using ADB.

In an aspect, the present disclosure provides a method for treating lung cancer in a patient having a lung nodule. The method can include, any one of, any combination of, or all of steps a″″, b″″, c″″ and d″″. Step a″″, can include obtaining a data set containing i) gene expression measurements of a biological sample from the patient, of at least two lung disease-associated genes, and ii) optionally clinical characteristics data of one or more clinical characteristics selected from the group of clinical characteristics listed in Table 6 of the patient. Step b″″, can include providing the data set as input to a machine-learning model trained to generate an inference of whether the data set is indicative of a malignant lung nodule or a benign lung nodule. Step c″″, can include receiving, as an output of the machine-learning model, the inference indicating whether the data set is indicative of the malignant lung nodule or the benign lung nodule. Step d″″, can include administering a treatment based on the patient's lung nodule being classified as a malignant nodule.

The data set of step a″″, can contain i) gene expression measurements of the biological sample from the patient, of at least two lung disease-associated genes selected from the group of genes listed in in any one or more of Tables 1, 2, 3, 4, 5, 7, and 8, and ii) optionally clinical characteristics data of one or more clinical characteristics selected from the group of clinical characteristics listed in Table 6 of the patient. In some embodiments, the at least two lung disease-associated genes of the data set of step a″″, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, or 182, or any value or range there between, genes selected from the group of genes listed in Table 1. In some embodiments, the at least two lung disease-associated genes of the data set of step a″″, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, or 175, or any value or range there between, genes selected from the group of genes listed in Table 2. In some embodiments, the at least two lung disease-associated genes of the data set of step a″″, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 or 62, or any value or range there between, genes selected from the group of genes listed in Table 3. In some embodiments, the at least two lung disease-associated genes of the data set of step a″″, include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295, or any value or range there between, genes selected from the group of genes listed in Table 4. In some embodiments, the at least two lung disease-associated genes of the data set of step a″″, include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, or 142, or any value or range there between, genes selected from the group of genes listed in Table 5. In some embodiments, the at least two lung disease-associated genes of the data set of step a″″, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7. In some embodiments, the at least two lung disease-associated genes of the data set of step a″″, are selected from BCAT1, CRCP, COA4, OVCA2, POM121, HLA-DPA1, VPS37C, MGST2, RNF220, HDAC3, NFE2L1, WDR20, CNPY4, HOXB2, C6orf120, TMEM8A, ASAP1-IT2, C15orf54, CD101, FNBP1, TECR, PROK2, SLC35B3, TDRD9, CLHC1, LPL, IFITM3, OGFOD3, EIF2B3, TMEM65, and MKRN3. In some embodiments, the at least two lung disease-associated genes of the data set of step a″″, include at least 2, 3, 4, 5, 6, 7, 8, 9, 10, 11, 12, 13, 14, 15, 16, 17, 18, 19, 20, or 21 genes selected from the group of genes listed in Table 8. In some embodiments, the at least two lung disease-associated genes of step a″″, are selected from BCAT1, USP32P2, CD177, QPCT, SCAF4, SNRPD3, BCL9L, THBS1, SLC22A18AS, ARCN1, DHX16, SATB1, ST6GAL1, CXCL1, TDRD9, ZNF831, MTCH1, FAM86HP, DHX8, RNF114, and DCTN4. In some embodiments, the one or more clinical characteristics of the data set of step a″″, include 1, 2, 3, 4, 5, 6, 7 or 8 clinical characteristics selected from the group of clinical characteristics listed in Table 6. In some embodiments, the one or more clinical characteristics of the data set of step a″″, include size of the nodule, age of the patient, presence of the nodule in the lung upper lobe, or any combination thereof. In some embodiments, the data set of step a″″, contains i) gene expression measurements of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7, and ii) clinical characteristics data of 1, 2, 3, 4, 5, 6, 7 or 8 clinical characteristics selected from the group of clinical characteristics listed in Table 6, of the patient. In some embodiments, the data set of step a″″, contains i) gene expression measurements of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7, and ii) clinical characteristics data of the clinical characteristics of the patient selected from size of the nodule, age of the patient, presence of the nodule in the lung upper lobe, or any combination thereof. In some embodiments, the at least two lung disease-associated genes of step a″″, comprise the 31 genes listed in Table 7, and the one or more clinical characteristics of step a″″ comprise the size of the nodule, age of the patient, and the presence of the nodule in the lung upper lobe. In some embodiments, the at least two lung disease-associated genes of step a″″, consist of the 31 genes listed in Table 7, and the one or more clinical characteristics of step a″″ consist of the size of the nodule, age of the patient, and the presence of the nodule in the lung upper lobe.

The inference from the machine learning model can include a confidence value between 0 and 1, such as, 0, 0.1, 0.2, 0.3, 0.4, 0.5, 0.6, 0.7, 0.8, 0.9 or 1, or any value or range there between, that the nodule is malignant, where higher confidence values may be correlated with a higher likelihood that the nodule is malignant. In some embodiments, the biological sample is a blood sample, isolated peripheral blood mononuclear cells (PBMCs), a lung biopsy sample, nasal fluid, saliva, or any derivative thereof. In some embodiments, the biological sample is a blood sample or any derivative thereof. In some embodiments, the biological sample is isolated peripheral blood mononuclear cells (PBMCs) or any derivative thereof. In some embodiments, the biological sample is a lung biopsy sample or any derivative thereof. In some embodiments, the biological sample is a nasal fluid sample or any derivative thereof. In some embodiments, the biological sample is a saliva sample or any derivative thereof. In certain embodiments, the method includes optionally performing biopsy of the lung nodule of the patient based at least in part on the classification of the lung nodule of the patient as the malignant lung nodule or the benign lung nodule. In certain embodiments, the method includes optionally performing a biopsy of the lung nodule of the patient based at least in part on the classification of the lung nodule of the patient as the malignant lung nodule. The decision to perform biopsy may depend on confidence value of the inference. The machine-learning model, e.g. of step b″″, can generate the inference of whether the data set is indicative of the patient having a malignant lung nodule or a benign lung nodule, wherein the patient having malignant lung nodule may indicate the patient has lung cancer, and the patient having benign lung nodule may indicate the patient does not have lung cancer. In certain embodiments, biopsy of the lung nodule of the patient is not performed. The machine-learning model of step b″″, can be trained according to a method described herein, e.g. according to the methods training of the machine-learning model of step b′.

The machine learning model of step b″″, can infer whether the data set is indicative of the patient having the malignant lung nodule or benign lung nodule with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model of step b″″, can infer whether the data set is indicative of the patient having the malignant lung nodule or benign lung nodule with a sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model of step b″″, can infer whether the data set is indicative of the patient having the malignant lung nodule or benign lung nodule with a specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model of step b″″, can infer whether the data set is indicative of the patient having the malignant lung nodule or benign lung nodule with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model of step b″″, can infer whether the data set is indicative of the patient having the malignant lung nodule or benign lung nodule with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model of step b″″, can infer whether the data set is indicative of the patient having the malignant lung nodule or benign lung nodule with a receiver operating characteristic (ROC) curve with an Area-Under-Curve (AUC) of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

The machine learning model of step b″″, can infer whether the data set is indicative of the patient having the malignant lung nodule or benign lung nodule with an accuracy of about 80% to about 100%. The machine learning model of step b″″, can infer whether the data set is indicative of the patient having the malignant lung nodule or benign lung nodule with an accuracy of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The machine learning model of step b″″, can infer whether the data set is indicative of the patient having the malignant lung nodule or benign lung nodule with an accuracy of about 80%, about 85%, about 90%, about 92%, about 94%, about 95%, about 96%, about 97%, about 98%, about 99%, about 99.5%, or about 100%. The machine learning model of step b″″, can infer whether the data set is indicative of the patient having the malignant lung nodule or benign lung nodule with an accuracy of at least about 80%, about 85%, about 90%, about 92%, about 94%, about 95%, about 96%, about 97%, about 98%, about 99%, or about 99.5%. The machine learning model of step b″″, can infer whether the data set is indicative of the patient having the malignant lung nodule or benign lung nodule with an accuracy of at most about 85%, about 90%, about 92%, about 94%, about 95%, about 96%, about 97%, about 98%, about 99%, about 99.5%, or about 100%. The machine learning model of step b″″, can infer whether the data set is indicative of the patient having the malignant lung nodule or benign lung nodule with a sensitivity of about 80% to about 100%. The machine learning model of step b″″, can infer whether the data set is indicative of the patient having the malignant lung nodule or benign lung nodule with a sensitivity of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The machine learning model of step b″″, can infer whether the data set is indicative of the patient having the malignant lung nodule or benign lung nodule with a sensitivity of about 80%, about 85%, about 90%, about 92%, about 94%, about 95%, about 96%, about 97%, about 98%, about 99%, about 99.5%, or about 100%. The machine learning model of step b″″, can infer whether the data set is indicative of the patient having the malignant lung nodule or benign lung nodule with a sensitivity of at least about 80%, about 85%, about 90%, about 92%, about 94%, about 95%, about 96%, about 97%, about 98%, about 99%, or about 99.5%. The machine learning model of step b″″, can infer whether the data set is indicative of the patient having the malignant lung nodule or benign lung nodule with a sensitivity of at most about 85%, about 90%, about 92%, about 94%, about 95%, about 96%, about 97%, about 98%, about 99%, about 99.5%, or about 100%. The machine learning model of step b″″, can infer whether the data set is indicative of the patient having the malignant lung nodule or benign lung nodule with a specificity of about 80% to about 100%. The machine learning model of step b″″, can infer whether the data set is indicative of the patient having the malignant lung nodule or benign lung nodule with a specificity of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The machine learning model of step b″″, can infer whether the data set is indicative of the patient having the malignant lung nodule or benign lung nodule with a specificity of about 80%, about 85%, about 90%, about 92%, about 94%, about 95%, about 96%, about 97%, about 98%, about 99%, about 99.5%, or about 100%. The machine learning model of step b″″, can infer whether the data set is indicative of the patient having the malignant lung nodule or benign lung nodule with a specificity of at least about 80%, about 85%, about 90%, about 92%, about 94%, about 95%, about 96%, about 97%, about 98%, about 99%, or about 99.5%. The machine learning model of step b″″, can infer whether the data set is indicative of the patient having the malignant lung nodule or benign lung nodule with a specificity of at most about 85%, about 90%, about 92%, about 94%, about 95%, about 96%, about 97%, about 98%, about 99%, about 99.5%, or about 100%. The machine learning model of step b″″, can infer whether the data set is indicative of the patient having the malignant lung nodule or benign lung nodule with a positive predictive value of about 80% to about 100%. The machine learning model of step b″″, can infer whether the data set is indicative of the patient having the malignant lung nodule or benign lung nodule with a positive predictive value of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The machine learning model of step b″″, can infer whether the data set is indicative of the patient having the malignant lung nodule or benign lung nodule with a positive predictive value of about 80%, about 85%, about 90%, about 92%, about 94%, about 95%, about 96%, about 97%, about 98%, about 99%, about 99.5%, or about 100%. The machine learning model of step b″″, can infer whether the data set is indicative of the patient having the malignant lung nodule or benign lung nodule with a positive predictive value of at least about 80%, about 85%, about 90%, about 92%, about 94%, about 95%, about 96%, about 97%, about 98%, about 99%, or about 99.5%. The machine learning model of step b″″, can infer whether the data set is indicative of the patient having the malignant lung nodule or benign lung nodule with a positive predictive value of at most about 85%, about 90%, about 92%, about 94%, about 95%, about 96%, about 97%, about 98%, about 99%, about 99.5%, or about 100%. The machine learning model of step b″″, can infer whether the data set is indicative of the patient having the malignant lung nodule or benign lung nodule with a negative predictive value of about 80% to about 100%. The machine learning model of step b″″, can infer whether the data set is indicative of the patient having the malignant lung nodule or benign lung nodule with a negative predictive value of about 80% to about 85%, about 80% to about 90%, about 80% to about 92%, about 80% to about 94%, about 80% to about 95%, about 80% to about 96%, about 80% to about 97%, about 80% to about 98%, about 80% to about 99%, about 80% to about 99.5%, about 80% to about 100%, about 85% to about 90%, about 85% to about 92%, about 85% to about 94%, about 85% to about 95%, about 85% to about 96%, about 85% to about 97%, about 85% to about 98%, about 85% to about 99%, about 85% to about 99.5%, about 85% to about 100%, about 90% to about 92%, about 90% to about 94%, about 90% to about 95%, about 90% to about 96%, about 90% to about 97%, about 90% to about 98%, about 90% to about 99%, about 90% to about 99.5%, about 90% to about 100%, about 92% to about 94%, about 92% to about 95%, about 92% to about 96%, about 92% to about 97%, about 92% to about 98%, about 92% to about 99%, about 92% to about 99.5%, about 92% to about 100%, about 94% to about 95%, about 94% to about 96%, about 94% to about 97%, about 94% to about 98%, about 94% to about 99%, about 94% to about 99.5%, about 94% to about 100%, about 95% to about 96%, about 95% to about 97%, about 95% to about 98%, about 95% to about 99%, about 95% to about 99.5%, about 95% to about 100%, about 96% to about 97%, about 96% to about 98%, about 96% to about 99%, about 96% to about 99.5%, about 96% to about 100%, about 97% to about 98%, about 97% to about 99%, about 97% to about 99.5%, about 97% to about 100%, about 98% to about 99%, about 98% to about 99.5%, about 98% to about 100%, about 99% to about 99.5%, about 99% to about 100%, or about 99.5% to about 100%. The machine learning model of step b″″, can infer whether the data set is indicative of the patient having the malignant lung nodule or benign lung nodule with a negative predictive value of about 80%, about 85%, about 90%, about 92%, about 94%, about 95%, about 96%, about 97%, about 98%, about 99%, about 99.5%, or about 100%. The machine learning model of step b″″, can infer whether the data set is indicative of the patient having the malignant lung nodule or benign lung nodule with a negative predictive value of at least about 80%, about 85%, about 90%, about 92%, about 94%, about 95%, about 96%, about 97%, about 98%, about 99%, or about 99.5%. The machine learning model of step b″″, can infer whether the data set is indicative of the patient having the malignant lung nodule or benign lung nodule with a negative predictive value of at most about 85%, about 90%, about 92%, about 94%, about 95%, about 96%, about 97%, about 98%, about 99%, about 99.5%, or about 100%. The machine learning model of step b″″, can infer whether the data set is indicative of the patient having the malignant lung nodule or benign lung nodule with a ROC curve with an AUC of about 0.8 to about 1. The machine learning model of step b″″, can infer whether the data set is indicative of the patient having the malignant lung nodule or benign lung nodule with a ROC curve with an AUC of about 0.8 to about 0.85, about 0.8 to about 0.9, about 0.8 to about 0.92, about 0.8 to about 0.94, about 0.8 to about 0.95, about 0.8 to about 0.96, about 0.8 to about 0.97, about 0.8 to about 0.98, about 0.8 to about 0.99, about 0.8 to about 0.995, about 0.8 to about 1, about 0.85 to about 0.9, about 0.85 to about 0.92, about 0.85 to about 0.94, about 0.85 to about 0.95, about 0.85 to about 0.96, about 0.85 to about 0.97, about 0.85 to about 0.98, about 0.85 to about 0.99, about 0.85 to about 0.995, about 0.85 to about 1, about 0.9 to about 0.92, about 0.9 to about 0.94, about 0.9 to about 0.95, about 0.9 to about 0.96, about 0.9 to about 0.97, about 0.9 to about 0.98, about 0.9 to about 0.99, about 0.9 to about 0.995, about 0.9 to about 1, about 0.92 to about 0.94, about 0.92 to about 0.95, about 0.92 to about 0.96, about 0.92 to about 0.97, about 0.92 to about 0.98, about 0.92 to about 0.99, about 0.92 to about 0.995, about 0.92 to about 1, about 0.94 to about 0.95, about 0.94 to about 0.96, about 0.94 to about 0.97, about 0.94 to about 0.98, about 0.94 to about 0.99, about 0.94 to about 0.995, about 0.94 to about 1, about 0.95 to about 0.96, about 0.95 to about 0.97, about 0.95 to about 0.98, about 0.95 to about 0.99, about 0.95 to about 0.995, about 0.95 to about 1, about 0.96 to about 0.97, about 0.96 to about 0.98, about 0.96 to about 0.99, about 0.96 to about 0.995, about 0.96 to about 1, about 0.97 to about 0.98, about 0.97 to about 0.99, about 0.97 to about 0.995, about 0.97 to about 1, about 0.98 to about 0.99, about 0.98 to about 0.995, about 0.98 to about 1, about 0.99 to about 0.995, about 0.99 to about 1, or about 0.995 to about 1. The machine learning model of step b″″, can infer whether the data set is indicative of the patient having the malignant lung nodule or benign lung nodule with a ROC curve with an AUC of about 0.8, about 0.85, about 0.9, about 0.92, about 0.94, about 0.95, about 0.96, about 0.97, about 0.98, about 0.99, about 0.995, or about 1. The machine learning model of step b″″, can infer whether the data set is indicative of the patient having the malignant lung nodule or benign lung nodule with a ROC curve with an AUC of at least about 0.8, about 0.85, about 0.9, about 0.92, about 0.94, about 0.95, about 0.96, about 0.97, about 0.98, about 0.99, or about 0.995. The machine learning model of step b″″, can infer whether the data set is indicative of the patient having the malignant lung nodule or benign lung nodule with a ROC curve with an AUC of at most about 0.85, about 0.9, about 0.92, about 0.94, about 0.95, about 0.96, about 0.97, about 0.98, about 0.99, about 0.995, or about 1.

In some embodiments, the treatment is configured to treat a lung cancer of the patient. In some embodiments, the treatment is configured to reduce a severity of a lung cancer of the patient. In some embodiments, the treatment is configured to reduce a risk of having a lung cancer of the patient. The treatment can include one or more treatments of lung cancer. In some embodiments, the treatment is surgery, chemotherapy, targeted therapy, immunotherapy, radiotherapy, or any combination thereof.

In an aspect, the present disclosure provides a method for assessing a lung nodule of a patient, for biopsy. The method can include, any one of, any combination of, or all of steps w, x, y and z. Step w, can include obtaining a data set containing i) gene expression measurements of a biological sample from the patient, of at least two lung disease-associated genes, and ii) optionally clinical characteristics data of one or more clinical characteristics selected from the group of clinical characteristics listed in Table 6 of the patient. The gene expression measurements can be obtained by assaying the biological sample. Step x, can include providing the data set as an input to a machine learning model trained to generate an inference of whether the data set is indicative of a malignant lung nodule or a benign lung nodule. Step y, can include receiving, as an output of the machine-learning model, the inference indicating whether the data set is indicative of the malignant lung nodule or the benign lung nodule. Step z, can include performing biopsy of the lung nodule based on the machine learning classification of the lung nodule. In some embodiments step z, can include performing biopsy of the lung nodule based on the lung nodule being classified as a malignant nodule or benign nodule. In some embodiments step z, can include performing biopsy of the lung nodule based on the lung nodule being classified as a malignant nodule. The decision to perform biopsy may depend on confidence value of the inference. In certain embodiments, biopsy of the lung nodule of the patient is not performed. In some embodiments, the data set of step w, contains i) gene expression measurements of the biological sample of at least two lung disease-associated genes selected from the group of genes listed in any one or more of Tables 1, 2, 3, 4, 5, 7, and 8, and ii) optionally clinical characteristics data of one or more clinical characteristics of the patient selected from the group of clinical characteristics listed in Table 6.

In some embodiments, the at least two lung disease-associated genes of the data set of step w, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, or 182, or any value or range there between, genes selected from the group of genes listed in Table 1. In some embodiments, the at least two lung disease-associated genes of the data set of step w, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, or 175, or any value or range there between, genes selected from the group of genes listed in Table 2. In some embodiments, the at least two lung disease-associated genes of the data set of step w, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 or 62, or any value or range there between, genes selected from the group of genes listed in Table 3. In some embodiments, the at least two lung disease-associated genes of the data set of step w, include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295, or any value or range there between, genes selected from the group of genes listed in Table 4. In some embodiments, the at least two lung disease-associated genes of the data set of step w, include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, or 142, or any value or range there between, genes selected from the group of genes listed in Table 5. In some embodiments, the at least two lung disease-associated genes of the data set of step w, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7. In some embodiments, the at least two lung disease-associated genes of step w, are selected from BCAT1, CRCP, COA4, OVCA2, POM121, HLA-DPA1, VPS37C, MGST2, RNF220, HDAC3, NFE2L1, WDR20, CNPY4, HOXB2, C6orf120, TMEM8A, ASAP1-IT2, C15orf54, CD101, FNBP1, TECR, PROK2, SLC35B3, TDRD9, CLHC1, LPL, IFITM3, OGFOD3, EIF2B3, TMEM65, and MKRN3. In some embodiments, the at least two lung disease-associated genes of the data set of step w, include at least 2, 3, 4, 5, 6, 7, 8, 9, 10, 11, 12, 13, 14, 15, 16, 17, 18, 19, 20, or 21 genes selected from the group of genes listed in Table 8. In some embodiments, the at least two lung disease-associated genes of step w, are selected from BCAT1, USP32P2, CD177, QPCT, SCAF4, SNRPD3, BCL9L, THBS1, SLC22A18AS, ARCN1, DHX16, SATB1, ST6GAL1, CXCL1, TDRD9, ZNF831, MTCH1, FAM86HP, DHX8, RNF114, and DCTN4. In some embodiments, the one or more clinical characteristics of the data set of step w, includes 1, 2, 3, 4, 5, 6, 7 or 8 clinical characteristics selected from the group listed in Table 6, of the patient. In some embodiments, one or more clinical characteristics of the data set of step w, includes size of the nodule, age of the patient, presence of the nodule in the lung upper lobe, or any combination thereof. In some embodiments, the data set of step w, contains i) gene expression measurements of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease-associated genes selected from the group of genes listed in Table 7, and ii) clinical characteristics data of 1, 2, 3, 4, 5, 6, 7 or 8 clinical characteristics selected from the group of clinical characteristics listed in Table 6. In some embodiments, the data set of step w, contains i) gene expression measurements of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease-associated genes selected from the group of genes listed in Table 7, and ii) clinical characteristics data of clinical characteristics of the patient selected from size of the nodule, age of the patient, presence of the nodule in the lung upper lobe, or any combination thereof. In some embodiments, the at least two lung disease-associated genes of step w, comprise the 31 genes listed in Table 7, and the one or more clinical characteristics of step w, comprise the size of the nodule, age of the patient, and the presence of the nodule in the lung upper lobe. In some embodiments, the at least two lung disease-associated genes of step w, consist of the 31 genes listed in Table 7, and the one or more clinical characteristics of step w, consist of the size of the nodule, age of the patient, and the presence of the nodule in the lung upper lobe.

In some embodiments, the biological sample is a blood sample, isolated peripheral blood mononuclear cells (PBMCs), a lung biopsy sample, nasal fluid, saliva, or any derivative thereof. In some embodiments, the biological sample is a blood sample or any derivative thereof. In some embodiments, the biological sample is isolated peripheral blood mononuclear cells (PBMCs) or any derivative thereof. In some embodiments, the biological sample is a lung biopsy sample, or any derivative thereof. In some embodiments, the biological sample is a nasal fluid sample, or any derivative thereof. In some embodiments, the biological sample is a saliva sample, or any derivative thereof.

The machine-learning model, e.g. of step x, can be trained according to a method described herein, e.g. according to the methods training of the machine-learning model of step b′.

Certain aspects are directed to a method for determining lung cancer in a patient. The method can include, any one of, any combination of, or all of steps w′, x′, y′ and z′. Step w′ can include obtaining a data set containing i) gene expression measurements of a biological sample from the patient, of at least two lung disease-associated genes, and ii) optionally clinical characteristics data of one or more clinical characteristics of the patient, selected from a group of clinical characteristics listed in Table 6. The gene expression measurements can be obtained by assaying the biological sample. Step x′ can include providing the data set as an input to a machine-learning model trained to generate an inference of whether the data set is indicative of the patient having or not having lung cancer. Step y′ can include receiving, as an output of the machine-learning model, the inference indicating whether the data set is indicative of the patient having or not having lung cancer. Step z′ can include electronically outputting a report indicating the patient has, or does not have lung cancer. The gene expression measurement of the biological sample can be performed using any suitable technique, such as any suitable RNA quantification technique, including but not limited to RNA-seq, Ampli-seq, or the like. In some embodiments, the data set of step w′, contains i) gene expression measurements of the biological sample of at least two lung disease-associated genes selected from the group of genes listed in any one or more of Tables 1, 2, 3, 4, 5, 7, and 8, and ii) optionally clinical characteristics data of one or more clinical characteristics of the patient selected from the group of clinical characteristics listed in Table 6.

In some embodiments, the at least two lung disease-associated genes of the data set of step w′, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, or 182, or any value or range there between, genes selected from the group of genes listed in Table 1. In some embodiments, the at least two lung disease-associated genes of the data set of step w′, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, or 175, or any value or range there between, genes selected from the group of genes listed in Table 2. In some embodiments, the at least two lung disease-associated genes of the data set of step w′, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 or 62, or any value or range there between, genes selected from the group of genes listed in Table 3. In some embodiments, the at least two lung disease-associated genes of the data set of step w′, include at least, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295, or any value or range there between, genes selected from the group of genes listed in Table 4. In some embodiments, the at least two lung disease-associated genes of the data set of step w′, include at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, or 142 genes selected from the group of genes listed in Table 5. In some embodiments, the at least two lung disease-associated genes of the data set of step w′, include includes at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 genes selected from the group of genes listed in Table 7. In some embodiments, the at least two lung disease-associated genes of step w′, are selected from BCAT1, CRCP, COA4, OVCA2, POM121, HLA-DPA1, VPS37C, MGST2, RNF220, HDAC3, NFE2L1, WDR20, CNPY4, HOXB2, C6orf120, TMEM8A, ASAP1-IT2, C15orf54, CD101, FNBP1, TECR, PROK2, SLC35B3, TDRD9, CLHC1, LPL, IFITM3, OGFOD3, EIF2B3, TMEM65, and MKRN3. In some embodiments, the at least two lung disease-associated genes of the data set of step w′, include at least 2, 3, 4, 5, 6, 7, 8, 9, 10, 11, 12, 13, 14, 15, 16, 17, 18, 19, 20, or 21 genes selected from the group of genes listed in Table 8. In some embodiments, the at least two lung disease-associated genes of step w, are selected from BCAT1, USP32P2, CD177, QPCT, SCAF4, SNRPD3, BCL9L, THBS1, SLC22A18AS, ARCN1, DHX16, SATB1, ST6GAL1, CXCL1, TDRD9, ZNF831, MTCH1, FAM86HP, DHX8, RNF114, and DCTN4 In some embodiments, the one or more clinical characteristics of the dataset of step w′, includes, 1, 2, 3, 4, 5, 6, 7 or 8 clinical characteristics selected from the group listed in Table 6. In some embodiments, the one or more clinical characteristics of the dataset of step w′, includes size of the nodule. In some embodiments, the one or more clinical characteristics of the dataset of step w′, includes age of the patient. In some embodiments, the one or more clinical characteristics of the dataset of step w′, includes presence of the nodule in the lung upper lobe. In some embodiments, the one or more clinical characteristics of the dataset of step w′, include size of the nodule, age of the patient, presence of the nodule in the lung upper lobe, or any combination thereof. In some embodiments, the data set of step w′, contains i) gene expression measurements of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease associated genes selected from the group of genes listed in Table 7, and ii) clinical characteristics data of 1, 2, 3, 4, 5, 6, 7 or 8 clinical characteristics of the patient selected from the group of clinical characteristics listed in Table 6. In some embodiments, the data set of step w′, contains i) gene expression measurements of the biological sample of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease-associated genes selected from the group of genes listed in Table 7, and ii) clinical characteristics data of the clinical characteristics of the patient selected from size of the nodule, age of the patient, presence of the nodule in the lung upper lobe, or any combination thereof. In some embodiments, the at least two lung disease-associated genes of step w′, comprise the 31 genes listed in Table 7, and the one or more clinical characteristics of step w′, comprise the size of the nodule, age of the patient, and the presence of the nodule in the lung upper lobe. In some embodiments, the at least two lung disease-associated genes of step w′, consist of the 31 genes listed in Table 7, and the one or more clinical characteristics of step w′, consist of the size of the nodule, age of the patient, and the presence of the nodule in the lung upper lobe.

In some embodiments, the biological sample is selected from the group: a blood sample, isolated peripheral blood mononuclear cells (PBMCs), a lung biopsy sample, nasal fluid, saliva, and any derivative thereof. In some embodiments, the biological sample is a blood sample or any derivative thereof. In some embodiments, the biological sample is isolated peripheral blood mononuclear cells (PBMCs) or any derivative thereof. In some embodiments, the biological sample is a lung biopsy sample, or any derivative thereof. In some embodiments, the biological sample is a nasal fluid sample, or any derivative thereof. In some embodiments, the biological sample is a saliva sample, or any derivative thereof.

The method can determine whether the patient has or does not have lung cancer with an accuracy of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The method can determine whether the patient has or does not have lung cancer with an sensitivity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The method can determine whether the patient has or does not have lung cancer with an specificity of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The method can determine whether the patient has or does not have lung cancer with a positive predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The method can determine whether the patient has or does not have lung cancer with a negative predictive value of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%. The machine learning model, e.g. of step x′, can infer whether the data set is indicative of the patient having or not having lung cancer with a receiver operating characteristic (ROC) curve with an AUC of at least about 0.50, at least about 0.55, at least about 0.60, at least about 0.65, at least about 0.70, at least about 0.75, at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

The inference from the machine learning model can include a confidence value between 0 and 1, such as, 0, 0.1, 0.2, 0.3, 0.4, 0.5, 0.6, 0.7, 0.8, 0.9 or 1, or any value or range there between, that the patient has lung cancer. Higher confidence values may be correlated with a higher likelihood that the patient has is lung cancer.

The machine-learning model, e.g. of step x′, can generate inference of whether the data set is indicative of the patient having a malignant lung nodule or a benign lung nodule, wherein the patient having malignant lung nodule may indicate that the patient has lung cancer, and patient having benign lung nodule may indicate that the patient does not have lung cancer. The machine-learning model can be trained according to a method described herein, e.g. according to the methods of training of the machine learning model of step b′.

In another aspect, the present disclosure provides a computer system for assessing a lung nodule of a subject, comprising: a database or other suitable data storage system that is configured to store a data set; and one or more computer processors operatively coupled to the database, wherein the one or more computer processors are individually or collectively programmed to: (i) analyze the data set to classify the lung nodule of the subject as a malignant lung nodule or a benign lung nodule; (ii) electronically output a report indicative of the classification of the lung nodule of the subject as the malignant lung nodule or the benign lung nodule. Computer-implemented methods as described herein may be executed on computer systems such as those described above. For example, a computer system may comprise one or more processors and one or more memory units that collectively store computer-readable executable instructions that, as a result of execution, cause the one or more processors to collectively perform the programmed steps described above. A computer system as described herein may comprise an assay device communicatively coupled to a personal computer. The data set can be a data set described herein. In some embodiments, the dataset comprise a) gene expression data, wherein the gene expression data is obtained by assaying a biological sample obtained or derived from the subject to produce gene expression measurements of the biological sample from each of a plurality of lung disease-associated genomic loci, wherein the plurality of disease-associated genomic loci comprises at least one gene selected from the group of genes listed in Table 4. In some embodiments, the data set contains i) gene expression measurements of a biological sample from the subject of at least two lung disease-associated genes selected from the group of genes listed in any one or more of Tables 1, 2, 3, 4, 5, 7, and 8, and ii) optionally clinical characteristics data of one or more clinical characteristics of the subject selected from the group of clinical characteristics listed in Table 6. In some embodiments, the data set contains i) gene expression measurements of the biological sample of the subject of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease associated genes selected from the group of genes listed in Table 7, and ii) clinical characteristics data of 1, 2, 3, 4, 5, 6, 7 or 8 clinical characteristics of the subject selected from the group of clinical characteristics listed in Table 6. In some embodiments, the data set contains i) gene expression measurements of the biological sample of the subject of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease-associated genes selected from the group of genes listed in Table 7, and ii) clinical characteristics data of the clinical characteristics of the subject selected from size of the nodule, age of the subject, presence of the nodule in the lung upper lobe, or any combination thereof. The biological sample can be a biological sample described herein. In some embodiments, the data set comprises i) the 31 genes listed in Table 7, and ii) the one or more clinical characteristics of the subject selected from the size of the nodule, age of the patient, and the presence of the nodule in the lung upper lobe. In some embodiments, the data set consists of i) the 31 genes listed in Table 7, and ii) the one or more clinical characteristics of the subject selected from the size of the nodule, age of the patient, and the presence of the nodule in the lung upper lobe. The biological sample can be a biological sample described herein.

In some embodiments, the computer system further comprises an electronic display operatively coupled to the one or more computer processors, wherein the electronic display comprises a graphical user interface that is configured to display the report.

In another aspect, the present disclosure provides one or more non-transitory computer readable media collectively comprising machine-executable code that, upon execution by one or more computer processors, causes the one or more computer processors to perform a method for assessing a lung nodule of a subject, the method comprising: (a) assaying a biological sample obtained or derived from the subject to produce a data set; (b) analyzing the data set to classify the lung nodule of the subject as a malignant lung nodule or a benign lung nodule; and (c) electronically outputting a report indicative of the classification of the lung nodule of the subject as the malignant lung nodule or the benign lung nodule. The data set can be a data set described herein. In some embodiments, the dataset comprise gene expression measurements of the biological sample from each of a plurality of lung disease-associated genomic loci, wherein the plurality of disease-associated genomic loci comprises at least one gene selected from the group listed in Table 4. In some embodiments, the data set contains i) gene expression measurements of the biological sample from the subject of at least two lung disease-associated genes selected from the group of genes listed in any one or more of Tables 1, 2, 3, 4, 5, 7, and 8, and ii) optionally clinical characteristics data of one or more clinical characteristics of the subject selected from the group of clinical characteristics listed in Table 6. In some embodiments, the data set contains i) gene expression measurements of the biological sample of the subject of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease associated genes selected from the group of genes listed in Table 7, and ii) clinical characteristics data of 1, 2, 3, 4, 5, 6, 7 or 8 clinical characteristics of the subject selected from the group of clinical characteristics listed in Table 6. In some embodiments, the data set contains i) gene expression measurements of the biological sample of the subject of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 lung disease-associated genes selected from the group of genes listed in Table 7, and ii) clinical characteristics data of the clinical characteristics of the subject selected from size of the nodule, age of the subject, presence of the nodule in the lung upper lobe, or any combination thereof. In some embodiments, the data set comprises i) the 31 genes listed in Table 7, and ii) the one or more clinical characteristics of the subject selected from the size of the nodule, age of the patient, and the presence of the nodule in the lung upper lobe. In some embodiments, the data set consists of i) the 31 genes listed in Table 7, and ii) the one or more clinical characteristics of the subject selected from the size of the nodule, age of the patient, and the presence of the nodule in the lung upper lobe. The biological sample can be a biological sample described herein.

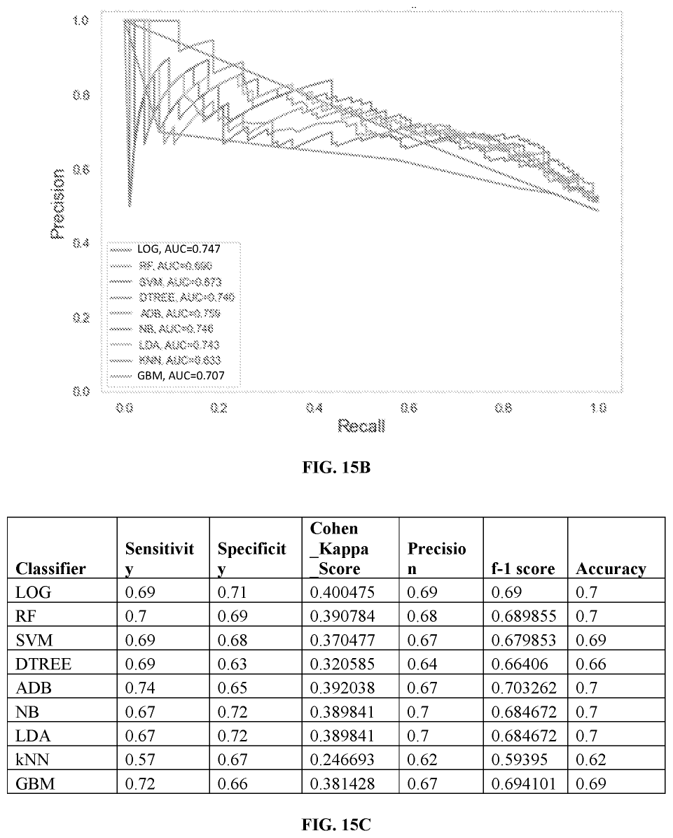

FIG. 10 illustrates an overview of an example method 1000 for assessing a lung nodule of a subject. The method 1000 may comprise assaying a biological sample obtained or derived from the subject to produce a data set comprising gene expression measurements of the biological sample at each of a plurality of lung disease-associated genomic loci, as in operation 1002. In some embodiments, the dataset further contains clinical characteristics data of one or more clinical characteristics selected from the group of clinical characteristic listed in Table 6 of the subject. In some embodiments, the plurality of disease-associated genomic loci comprises at least one gene selected from the group of genes listed in Table 1. In some embodiments, the plurality of disease-associated genomic loci comprises at least one gene selected from the group of genes listed in Table 2. In some embodiments, the plurality of disease-associated genomic loci comprises at least one gene selected from the group of genes listed in Table 3. In some embodiments, the plurality of disease-associated genomic loci comprises at least one gene selected from the group of genes listed in Table 4. In some embodiments, the plurality of disease-associated genomic loci comprises at least one gene selected from the group of genes listed in Table 5. In some embodiments, the plurality of disease-associated genomic loci comprises at least one gene selected from the group of genes listed in Table 7. In some embodiments, the plurality of disease-associated genomic loci comprises at least one gene selected from the group of genes listed in Table 8. In some embodiments, the data set comprises i) gene expression measurement of the biological sample from the patient of at least 2 lung disease-associated genes selected from the group of genes listed in Table 7, and clinical characteristics data of one or more clinical characteristics selected from the group of clinical characteristic listed in Table 6 of the subject. The method 1000 may comprise analyzing the data set to classify the lung nodule of the subject as a malignant lung nodule or a benign lung nodule, as in operation 1004. The method 1000 may comprise electronically outputting a report indicative of the classification of the lung nodule of the subject as the malignant lung nodule or the benign lung nodule, as in operation 1006.

Methods of the present disclosure may comprise applying a trained machine learning algorithm to gene expression data (e.g., acquired by RNA-Seq, Ampli-seq, or like) and optionally clinical characteristics data of a subject, to assess a lung nodule of the subject. The trained machine learning algorithm may comprise a machine learning based classifier, configured to process the gene expression data and optionally clinical characteristics data to assess the lung nodule (e.g., determine whether a lung nodule is malignant or benign). The machine learning classifier may be trained using clinical datasets, e.g. reference data sets from one or more cohorts of subjects, e.g., using gene expression data and/or clinical health data, e.g. clinical characteristics data of the subjects as inputs and known clinical health outcomes (e.g., a lung nodule that is malignant or benign) of the subjects as outputs to the machine learning classifier.

The machine learning classifier may comprise one or more machine learning algorithms. Examples of machine learning algorithms may include a linear regression, a logistic regression (LOG), a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, a linear discriminant analysis (LDA), a decision tree learning (DTREE), an adaptive boosting (ADB) or any combination thereof, or another supervised learning algorithm or unsupervised learning algorithm for classification and regression. The machine learning classifier may be trained using one or more reference datasets corresponding to subject data (e.g., gene expression data and/or clinical health data).

Reference datasets used for training machine learning classifiers, may be generated from, for example, one or more cohorts of patients having common clinical characteristics (features) and clinical outcomes (labels). Reference datasets may comprise a set of features and labels corresponding to the features. Features may correspond to algorithm inputs comprising subject data (e.g., gene expression data and/or clinical health data, e.g. clinical characteristics data). Features may comprise clinical characteristics such as, for example, certain ranges, categories, or levels of gene expression data and/or clinical health data. Features may comprise subject information such as patient age, patient medical history, other medical conditions, current or past medications, size of the nodule, presence of the nodule in the lung upper lobe and/or time since the last observation. For example, a set of features collected from a given patient at a given time point may collectively serve as a signature, which may be indicative of clinical health outcomes (e.g., a lung nodule that is malignant or benign) of the subject at the given time point.

For example, ranges of subject data (e.g., gene expression data and/or clinical health data) may be expressed as a plurality of disjoint continuous ranges of continuous measurement values, and categories of subject data (e.g., gene expression data and/or clinical health data) may be expressed as a plurality of disjoint sets of measurement values (e.g., {“high”, “low”}, {“high”, “normal”}, {“low”, “normal”}, {“high”, “borderline high”, “normal”, “low”}, {“Yes”, “No”}, {“Present”, “Absent”} etc.). Clinical characteristics may also include clinical labels indicating the subject's health history, such as a diagnosis of a disease or disorder, a previous administering of a clinical treatment (e.g., a drug, a surgical treatment, chemotherapy, radiotherapy, immunotherapy, etc.), behavioral factors, or other health status (e.g., hypertension or high blood pressure, hyperglycemia or high blood glucose, hypercholesterolemia or high blood cholesterol, history of allergic reaction or other adverse reaction, etc.). Clinical characteristics data for the clinical characteristic, AGE, of the patient can be age of the patient. Clinical characteristics data for the clinical characteristic, SEX, of the patient can be sex of the patient. Clinical characteristics data for the clinical characteristic, presence of the nodule in the lung upper lobe (NCNUPYN), of the patient can be yes or no. Clinical characteristics data for the clinical characteristic, smoking status (MHTBSTAT), of the patient can be past or current. Clinical characteristics data for the clinical characteristics, chronic obstructive pulmonary disease (MHCPDYN), of the patient can be yes or no. Clinical characteristics data for the clinical characteristics, lung nodule spiculated (NCNMYN), of the patient can be yes or no. Clinical characteristics data for the clinical characteristic, emphysemal (MHEMPYN), of the patient can be yes or no. Labels may comprise clinical outcomes such as, for example, a lung nodule that is malignant or benign.

The machine learning classifier algorithm may process the input features to generate output values comprising one or more classifications, one or more predictions, or a combination thereof. For example, such classifications or predictions may include a binary classification of a lung nodule, a classification between a group of categorical labels (e.g., ‘malignant lung nodule’ and ‘benign lung nodule’), a likelihood (e.g., relative likelihood or probability) of having a malignant lung nodule or benign lung nodule, and a confidence interval for any numeric predictions. Various machine learning techniques may be cascaded such that the output of a machine learning technique may also be used as input features to subsequent layers or subsections of the machine learning classifier.

In order to train the machine learning classifier model (e.g., by determining weights and correlations of the model) to generate real-time classifications or predictions, the model can be trained using reference datasets. Such datasets may be sufficiently large to generate statistically significant classifications or predictions. In some cases, datasets are annotated or labeled.

Datasets may be split into subsets (e.g., discrete or overlapping), such as a training dataset, a development dataset, and a test dataset. For example, a dataset may be split into a training dataset comprising 80% of the dataset, a development dataset comprising 10% of the dataset, and a test dataset comprising 10% of the dataset. The training dataset may comprise about 10%, about 20%, about 30%, about 40%, about 50%, about 60%, about 70%, about 80%, or about 90% of the dataset. The development dataset may comprise about 10%, about 20%, about 30%, about 40%, about 50%, about 60%, about 70%, about 80%, or about 90% of the dataset. The test dataset may comprise about 10%, about 20%, about 30%, about 40%, about 50%, about 60%, about 70%, about 80%, or about 90% of the dataset. Training sets (e.g., training datasets) may be selected by random sampling of a set of data corresponding to one or more patient cohorts to ensure independence of sampling. Alternatively, training sets (e.g., training datasets) may be selected by proportionate sampling of a set of data corresponding to one or more patient cohorts to ensure independence of sampling.

Reference datasets may be split into subsets (e.g., discrete or overlapping), such as a training dataset, and a validation dataset. For example, a reference dataset may be split into a training dataset containing 80% of the dataset, and a validation dataset containing 20% of the dataset. The training dataset may contain 5%, 10%, 20%, 30%, 40%, 50%, 60%, 70%, 80%, 90% or 95%, or any value or range there between, of the reference dataset. The validation dataset may contain 5%, 10%, 20%, 30%, 40%, 50%, 60%, 70%, 80%, 90% or 95%, or any value or range there between, of the reference dataset. 2, 2.5, 5 or 10, or any value or range there between, fold cross validation can be used.

To validate the performance of the machine learning classifier model, different performance metrics may be generated. For example, an area under the receiver-operating curve (AUROC) may be used to determine the diagnostic capability of the machine learning classifier. For example, the machine learning classifier may use classification thresholds which are adjustable, such that specificity and sensitivity are tunable, and the receiver-operating curve (ROC) can be used to identify the different operating points corresponding to different values of specificity and sensitivity.

In some cases, such as when datasets are not sufficiently large, cross-validation may be performed to assess the robustness of a machine learning classifier model across different training and testing datasets.

To calculate performance metrics such as sensitivity, specificity, accuracy, positive predictive value (PPV), negative predictive value (NPV), AUPRC, AUROC, or similar, the following definitions may be used. A “false positive” may refer to an outcome in which a lung nodule of a subject is incorrectly classified as a malignant lung nodule. A “true positive” may refer to an outcome in which a lung nodule of a subject is correctly classified as a malignant lung nodule. A “false negative” may refer to an outcome in which a lung nodule of a subject is incorrectly classified as a benign lung nodule. A “true negative” may refer to an outcome in which a lung nodule of a subject is correctly classified as a benign lung nodule.

The gene expression measurements can be performed using any suitable technique, such any suitable RNA quantification techniques, including but not limited to RNA-seq, Ampli-seq, or the like. In some embodiments, gene expression data is obtained by a data analysis tool selected from the group: a BIG-C™ big data analysis tool, an I-Scope™ big data analysis tool, a T-Scope™ big data analysis tool, a Cell Scan big data analysis tool, an MS (Molecular Signature) Scoring™ analysis tool, and a Gene Set Variation Analysis (GSVA) tool (e.g., P-Scope).

The machine learning classifier may be trained until certain predetermined conditions for accuracy or performance are satisfied, such as having minimum desired values corresponding to diagnostic accuracy measures. For example, the diagnostic accuracy measure may correspond to prediction of a likelihood of a lung nodule being malignant or benign. Examples of diagnostic accuracy measures may include sensitivity, specificity, positive predictive value (PPV), negative predictive value (NPV), accuracy, area under the precision-recall curve (AUPRC), and area under the curve (AUC) of a Receiver Operating Characteristic (ROC) curve (AUROC) corresponding to the diagnostic accuracy of determining whether a lung nodule is malignant or benign.

For example, such a predetermined condition may be that the sensitivity of determining whether a lung nodule is malignant or benign comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a predetermined condition may be that the specificity of determining whether a lung nodule is malignant or benign comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a predetermined condition may be that the positive predictive value (PPV) of determining whether a lung nodule is malignant or benign comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a predetermined condition may be that the negative predictive value (NPV) of determining whether a lung nodule is malignant or benign comprises a value of, for example, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

As another example, such a predetermined condition may be that the area under the curve (AUC) of a Receiver Operating Characteristic (ROC) curve (AUROC) of determining whether a lung nodule is malignant or benign comprises a value of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

As another example, such a predetermined condition may be that the area under the precision-recall curve (AUPRC) of determining whether a lung nodule is malignant or benign comprises a value of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained classifier may be trained or configured to determine whether a lung nodule is malignant or benign with a sensitivity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained classifier may be trained or configured to determine whether a lung nodule is malignant or benign with a specificity of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained classifier may be trained or configured to determine whether a lung nodule is malignant or benign with a positive predictive value (PPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained classifier may be trained or configured to determine whether a lung nodule is malignant or benign with a negative predictive value (NPV) of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, or at least about 99%.

In some embodiments, the trained classifier may be trained or configured to determine whether a lung nodule is malignant or benign with an area under the curve (AUC) of a Receiver Operating Characteristic (ROC) curve (AUROC) of at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

In some embodiments, the trained classifier may be trained or configured to determine whether a lung nodule is malignant or benign with an area under the precision-recall curve (AUPRC) of at least about 0.10, at least about 0.15, at least about 0.20, at least about 0.25, at least about 0.30, at least about 0.35, at least about 0.40, at least about 0.45, at least about 0.50, at least about 0.55, at least about 0.60, at least about 0.65, at least about 0.70, at least about 0.75, at least about 0.80, at least about 0.85, at least about 0.90, at least about 0.95, at least about 0.96, at least about 0.97, at least about 0.98, or at least about 0.99.

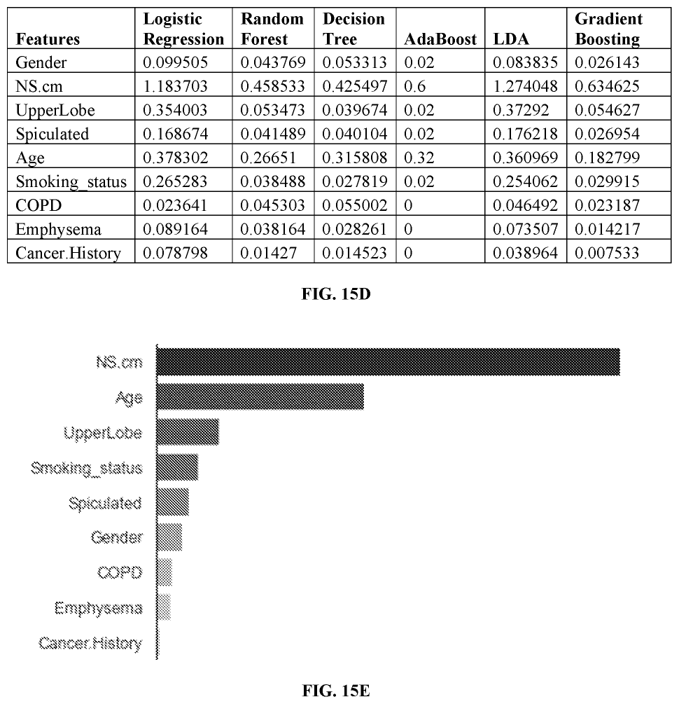

The present disclosure provides computer systems that are programmed to implement methods of the disclosure. FIG. 11 shows a computer system 1101 that is programmed or otherwise configured to implement methods provided herein.

The computer system 1101 can regulate various aspects of the present disclosure, such as, for example, assaying a biological sample obtained or derived from the subject to produce a data set comprising gene expression measurements of the biological sample from each of a plurality of lung disease-associated genomic loci, analyzing the data set to classify the lung nodule of the subject as a malignant lung nodule or a benign lung nodule, and electronically outputting a report indicative of the classification of the lung nodule of the subject as the malignant lung nodule or the benign lung nodule. In some embodiments, the data set further contains clinical characteristics data of one or more clinical characteristics selected from the group of clinical characteristics listed in Table 6 of the patient. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet.

The network 1130 in some cases is a telecommunication and/or data network. The network 1130 can include one or more computer servers, which can enable distributed computing, such as cloud computing. For example, one or more computer servers may enable cloud computing over the network 1130 (“the cloud”) to perform various aspects of analysis, calculation, and generation of the present disclosure, such as, for example, assaying a biological sample obtained or derived from the subject to produce a data set comprising gene expression measurements of the biological sample from each of a plurality of lung disease-associated genomic loci, analyzing the data set to classify the lung nodule of the subject as a malignant lung nodule or a benign lung nodule, and electronically outputting a report indicative of the classification of the lung nodule of the subject as the malignant lung nodule or the benign lung nodule. In some embodiments, the data set further contains clinical characteristics data of one or more clinical characteristics selected from the group of clinical characteristics listed in Table 6 of the patient. Such cloud computing may be provided by cloud computing platforms such as, for example, Amazon Web Services (AWS), Microsoft Azure, Google Cloud Platform, and IBM cloud. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140. Examples of user interfaces (UIs) include, without limitation, a graphical user interface (GUI) and web-based user interface. For example, the computer system can include a graphical user interface (GUI) configured to display, for example, subject data, identification of a lung nodule of the subject as a malignant lung nodule or a benign lung nodule, and/or predictions or assessments generated from subject data.

Methods and systems of the present disclosure can be implemented by way of one or more algorithms. An algorithm can be implemented by way of software upon execution by the central processing unit 1105. The algorithm can, for example, assay a biological sample obtained or derived from the subject to produce a data set comprising gene expression measurements of the biological sample at each of a plurality of lung disease-associated genomic loci, analyze the data set to classify the lung nodule of the subject as a malignant lung nodule or a benign lung nodule, and electronically output a report indicative of the classification of the lung nodule of the subject as the malignant lung nodule or the benign lung nodule. In some embodiments, the data set further contains clinical characteristics data of one or more clinical characteristics selected from the group of clinical characteristics listed in Table 6 of the patient.

### EXAMPLES

### Example 1: Machine Learning Classification of RNA-Seq Data

Differential gene expression analysis was performed to identify genes that were most differentially expressed (e.g., biomarkers) in whole blood samples between subjects having benign lung nodules and malignant lung nodules. A biomarker dataset comprising samples from 152 subjects was analyzed. Among those, 80 of the samples in the biomarker dataset had a diagnosis of a benign lung nodule, and 72 samples had a diagnosis of a malignant lung nodule. Gene expression measurements of whole blood samples from the subjects were analyzed using RNA-Seq technique.

A training dataset comprising lung nodule samples from 604 subjects was used to train a machine learning algorithm. Gene expression measurements of whole blood samples from the subjects were analyzed. Subsequently, a validation dataset comprising samples of long nodules from 487 subjects were used to validate the machine learning algorithm. The samples were analyzed using RNA-Seq techniques. In the following examples, eight machine learning classifiers including Gradient boosting machines (GBM), Logistic regression model (LOG), Support vector machines (SVM), Random forest (RF), Generalized linear model (GLM), k-nearest neighbors (kNN), Naïve Bayes (NB) and Elastic Networks (EN) were trained to distinguish malignant lung nodules versus benign lung nodules based on an analysis of the RNA-Seq data.

Eight different machine learning classifiers were trained to determine a high-performing set of genes to distinguish malignant lung nodules versus benign lung nodules using the biomarker dataset. The biomarker dataset was obtained by whole transcriptome RNA sequencing. The biomarker dataset comprised 80 lung nodule samples that had a diagnosis of a benign lung nodule and 72 samples that had a diagnosis of a malignant lung nodule.

A total of 1,430 genes were initially identified to be differentially expressed between malignant lung nodule samples and benign lung nodule samples. A Log2 ratio of gene expression of the differentially expressed genes was used to determine the optimal set of genes. The Log2 ratio was defined as T/R, where T is the gene expression level in the testing sample, and R is the gene expression level in the reference sample. After removing a subset of the 1,430 genes that exhibited collinear expression (correlation or r>0.8), a total number of 1,178 gene features (Table 9) were identified.

TABLE 9
Gene set of 1,178 gene features

| | | | | | | | |
|---|---|---|---|---|---|---|---|
| A2M-AS1 | CAPNS2 | EIF2B3 | HOXA9 | MECP2 | PITPNM2 | SEMA3G | TMEM165 |
| AAAS | CARD8-AS1 | EIF2B5 | HOXB2 | MED1 | PITRM1 | SEPN1 | TMEM170B |
| AARS | CC2D1A | EIF4ENIF1 | HP | MED28 | PITRM1-AS1 | 44450 | TMEM175 |
| AARS2 | CCAR1 | EIF4G1 | HRSP12 | MEST | PJA1 | SERP1 | TMEM189 |
| AASDHPPT | CCDC28A | EIF5 | HTATIP2 | METTL1 | PKD1P6 | SETD1A | TMEM192 |
| AATBC | CCDC64 | ELAC2 | HYLS1 | MFAP3 | PKHD1L1 | SETMAR | TMEM201 |
| ABCB1 | CCDC89 | ELAVL1 | HYOU1 | MFGE8 | PKP4 | SF3A2 | TMEM218 |
| ABCC13 | CCDC94 | ELP3 | IFFO2 | MFN1 | PLA2G4A | SF3B4 | TMEM56-RWDD3 |
| ABCC6 | CCDC97 | EMB | IFI27 | MFSD12 | PLA2G4C | SFSWAP | TMEM63C |
| ABCF1 | CCHCR1 | EMC1 | IFI44L | MGA | PLBD2 | SFTPD | TMEM65 |
| ABCF2 | CCL5 | EMC6 | IFITM3 | MGC16025 | PLCB1 | SGK494 | TMEM71 |
| ABHD15 | CCNB2 | EMD | IFT172 | MGC16275 | PLCH1 | SGSH | TMEM87A |
| ABHD3 | CCNF | EML3 | IFT27 | MGEA5 | PLCXD1 | SGSM1 | TMEM91 |
| ABHD6 | CCNG2 | ENTPD6 | IGSF8 | MGLL | PLD3 | SH3BP1 | TMOD2 |
| ABTB2 | CCNL1 | EOMES | IKZF4 | MGST2 | PLEKHA3 | SH3GL1P1 | TMPPE |
| ACACB | CCNT2-AS1 | EPM2AIP1 | IKZF5 | MICALL1 | PLEKHG2 | SH3TC2 | TMPRSS13 |
| ACCS | CCSAP | EPN2 | IL12RB1 | MIR22HG | PLOD3 | SIAH3 | TMPRSS9 |
| ACE | CD101 | ERICH6-AS1 | IL18 | MIR3939 | PLSCR1 | SIGLEC10 | TMX4 |
| ACLY | CD164 | ERRFI1 | INF2 | MIR5194 | PLVAP | SIMC1 | TNFAIP8L1 |
| ACSBG1 | CD177 | ESPN | ING1 | MIR7845 | POLD2 | SIPA1L3 | TNFRSF10B |
| ACSM3 | CD226 | ESYT1 | ING5 | MKI67 | POLR1A | SLA2 | TNKS2 |
| ACTN4 | CD58 | EXOC1 | INHBB | MKKS | POLR1B | SLAMF7 | TNNT1 |
| ACTR10 | CD84 | EXOSC10 | INO80 | MKLN1 | POLR2C | SLAMF8 | TNRC6A |
| ADAM9 | CDAN1 | EXOSC3 | INPPL1 | MKRN3 | POLR3D | SLC10A3 | TOR1A |
| ADAMTSL4 | CDC14B | EYA3 | IQCC | MLC1 | POM121 | SLC12A4 | TPCN1 |
| ADARB1 | CDC20 | F2R | IQCE | MLEC | POM121C | SLC16A13 | TPK1 |
| ADCY3 | CDC42EP4 | F8A1 | ITFG3 | MLLT6 | POMT1 | SLC17A9 | TPP1 |
| ADCY9 | CDC73 | FAM105A | ITGA10 | MOGS | POTEE | SLC1A7 | TPPP |
| ADGRG1 | CDHR1 | FAM160B2 | ITGA3 | MPL | POU2F2 | SLC22A15 | TPTEP1 |
| ADGRG5 | CDIP1 | FAM161B | ITGB1 | MRC2 | PPARA | SLC25A14 | TRAF2 |
| ADHFE1 | CDK5R1 | FAM182A | ITGB5 | MRPL23 | PPIL2 | SLC25A40 | TRAF3IP1 |
| ADTRP | CDKN1B | FAM193A | ITGB7 | MS4A3 | PPM1L | SLC25A45 | TRAM2 |
| AGAP1 | CDO1 | FAM198B | ITIH4 | MS4A4A | PPP1R15B | SLC27A4 | TRIM26 |
| AGER | CEBPA | FAM199X | ITPR1 | MSMO1 | PPP1R21 | SLC29A3 | TRIM62 |
| AGFG1 | CENPT | FAM200B | ITPRIPL1 | MTA2 | PPP1R3D | SLC2A3 | TRIO |
| AGFG2 | CEP104 | FAM217B | IVNS1ABP | MTFMT | PPP2R5A | SLC30A1 | TRMT10A |
| AGPAT4-IT1 | CEP164 | FAM78A | JAKMIP1 | MTM1 | PPP6C | SLC35B3 | TRMT1L |
| AGPAT9 | CEP250 | FAM86FP | JAKMIP2 | MUC20 | PRCP | SLC35F5 | TSEN34 |
| AHNAK | CEP295NL | FAM95C | JMJD7 | MUM1 | PRDM15 | SLC36A4 | TSHR |
| AIFM2 | CEP44 | FANCG | JOSD1 | MVB12B | PRDM4 | SLC37A3 | TSHZ1 |
| AKIRIN1 | CEP89 | FAS | KAT6B | MXD3 | PRDM5 | SLC38A2 | TSNAX |
| AKR1C1 | CFAP58-AS1 | FAT4 | KCNA2 | MYLK | PRDX3 | SLC46A1 | TSPAN33 |
| ALDH18A1 | CHCHD10 | FBRS | KDM2B | MYO15B | PRF1 | SLC47A1 | TSPAN9 |
| ALKBH6 | CHD3 | FBXL18 | KDM7A | MYOF | PRKACA | SLC4A4 | TSPYL2 |
| AMBRA1 | CHD8 | FBXO28 | KHSRP | MYOM2 | PROCA1 | SLC6A12 | TSTA3 |
| AMIGO3 | CHERP | FBXO33 | KIAA0100 | NAA60 | PROK2 | SLC8A3 | TTC33 |
| ANGPT1 | CHMP4A | FBXO38 | KIAA0195 | NACC1 | PRR5L | SLC9A1 | TTC38 |
| ANKRD17 | CHSY1 | FBXO46 | KIAA0556 | NAPB | PRSS33 | SLX4 | TTC7A |
| ANKRD42 | CKAP5 | FCAR | KIAA0825 | NAPG | PRSS35 | SMAD7 | TTYH2 |
| ANKRD50 | CLCC1 | FEZ1 | KIAA1211 | NBPF10 | PRX | SMARCA4 | TTYH3 |
| ANKS3 | CLDN15 | FGD2 | KIAA1683 | NCAPD2 | PSEN2 | SMARCD3 | TUBA1C |
| ANO6 | CLDN9 | FGF9 | KIF13B | NCAPD3 | PSMA1 | SMC2 | TUBA4B |
| ANPEP | CLEC16A | FGFBP2 | KIF3B | NCK2 | PSMC4 | SMG1P5 | TUT1 |
| ANXA3 | CLEC4D | FGFR4 | KIFC3 | NCKIPSD | PSMD5 | SMG9 | U2AF2 |
| AOC3 | CLEC5A | FGFRL1 | KIZ | NCR1 | PTAR1 | SMIM14 | UBA1 |
| AP1B1 | CLEC7A | FHOD1 | KLHDC2 | NCR3 | PTBP1 | SMIM8 | UBA7 |
| AP2A2 | CLHC1 | FIGNL1 | KLHDC4 | NCR3LG1 | PTCH1 | SMNDC1 | UBAP2L |
| AP3D1 | CLIC5 | FKBP11 | KLHDC8B | NDE1 | PTCH2 | SMPD3 | UBE2A |
| AP3S1 | CLIP2 | FKBP5 | KLHL25 | NDST2 | PTGDR | SMPDL3B | UBE2Q1 |
| AP4M1 | CLK4 | FLJ10038 | KPNA4 | NEK4 | PTGDS | SNAPC4 | UBXN11 |
| AP5M1 | CLPTM1 | FLJ26850 | KRBA1 | NFATC1 | PTGFR | SNORA18 | UCKL1 |
| AP5Z1 | CLSTN3 | FLJ37453 | KSR1 | NFATC3 | PTGS2 | SNORA25 | UCP2 |
| APOBEC3A | CNIH4 | FLJ41278 | KYNU | NFE2L1 | PTK7 | SNORA32 | UCP3 |
| APOBEC3F | CNNM4 | FLT3 | L3MBTL1 | NFKBIB | PTOV1-AS2 | SNORA38 | UGCG |
| APOL3 | CNOT3 | FMNL3 | L3MBTL2 | NHEJ1 | PTP4A1 | SNRNP200 | UHMK1 |
| APPBP2 | CNOT8 | FMR1 | LAIR1 | NID1 | PTPN18 | SNRPA | UMODL1-AS1 |
| ARFIP1 | CNPY4 | FNBP1 | LAMA2 | NLGN3 | PTPN23 | SNX33 | UNC45B |
| ARG1 | COA4 | FOXD2-AS1 | LAPTM4A | NMT1 | PTPN3 | SOCS7 | UPK3B |
| ARG2 | COA5 | FRMPD3 | LARP1 | NOL6 | PTPRA | SP2 | UQCC3 |
| ARHGAP21 | COL13A1 | FUT7 | LAS1L | NOMO1 | PTX3 | SPAG16 | USF2 |
| ARHGAP24 | COL6A1 | GABBR1 | LCAT | NOMO2 | PURB | SPAG5 | USP10 |
| ARHGAP32 | COL6A2 | GABPB1-AS1 | LEMD1-AS1 | NOP14 | PVRL2 | SPAG8 | USP28 |
| ARHGAP33 | COL6A3 | GABPB2 | LETM1 | NPC1 | PWP2 | SPATA5L1 | USP31 |
| ARHGEF1 | COLGALT2 | GADD45A | LIG3 | NPIPB11 | PYGB | SPCS3 | USP38 |
| ARHGEF10 | COMMD3 | GALNT3 | LILRA5 | NPIPB5 | PYGM | SPECC1L | USP54 |
| ARL15 | COQ2 | GALNT4 | LIMA1 | NPL | PYROXD2 | SPN | VANGL1 |
| ARL8B | COQ4 | GANAB | LINC00174 | NR112 | QRICH1 | SPNS1 | VARS |
| ARRDC3-AS1 | COX15 | GAREML | LINC00189 | NR2F6 | RAB10 | SPPL2A | VARS2 |
| ARRDC4 | CPEB2 | GATS | LINC00299 | NRAS | RAB14 | SPRTN | VCPKMT |
| ARRDC5 | CPNE3 | GBP6 | LINC00493 | NRGN | RABL2B | SPTBN5 | VENTX |
| ARSA | CRAMP1L | GCLC | LINC00598 | NRIR | RABL6 | SQRDL | VGLL4 |
| ASAP1-IT2 | CRCP | GCNT2 | LINC00671 | NRL | RACGAP1 | SRA1 | VIL1 |
| ASB7 | CRIM1 | GDI1 | LINC00909 | NRROS | RAD18 | SRC | VNN1 |
| ASMTL-AS1 | CRTC2 | GDPD5 | LINC00925 | NT5DC2 | RAD54L2 | SREBF1 | VPS25 |
| ATAD3B | CSF1R | GEMIN5 | LINC00944 | NT5M | RAI1 | SRP68 | VPS26A |
| ATG12 | CSGALNACT1 | GFOD1 | LINC00969 | NUDT4 | RAI2 | SRRT | VPS37C |
| ATN1 | CSGALNACT2 | GGT3P | LINC01001 | NUP188 | RANBP3 | ST20 | VPS52 |
| ATP13A3 | CSNK1A1 | GIGYF2 | LINC01002 | NUP210L | RAP1A | ST3GAL6 | VTA1 |
| ATP5D | CTNS | GINM1 | LINC01012 | NUP93 | RAP2C | ST6GALNAC3 | WASF3 |
| ATP8B4 | CTSA | GIPR | LINC01126 | NUTM2D | RAPGEF1 | ST8SIA6 | WDR11-AS1 |
| AUTS2 | CTSG | GLG1 | LINC01137 | OBFC1 | RAPGEFL1 | STIP1 | WDR20 |
| AVPR1A | CUL7 | GLRX | LINC01226 | ODF2 | RARA-AS1 | STK11IP | WDR45B |
| AXIN1 | CUTC | GNG10 | LINC01347 | OGFOD3 | RASA3 | STK25 | WDR46 |
| AZI2 | CWC27 | GOLGA1 | LINC01578 | OLFM2 | RASAL3 | STRAP | WDR60 |
| AZU1 | CX3CR1 | GOLGA2 | LINGO2 | OR52K2 | RAVER1 | STRIP1 | WDR81 |
| B3GNT5 | CXCL1 | GOLGA3 | LMF1 | ORAI3 | RB1 | STT3A | WHSC1 |
| B4GALT7 | CYP1B1 | GON4L | LOC100049716 | ORAOV1 | RBM10 | STX7 | WIZ |
| BAG4 | CYP2S1 | GOT2 | LOC100128239 | ORC4 | RBM12B | STYXL1 | WNT10B |
| BAHD1 | CYP4F12 | GP1BA | LOC100130093 | ORM1 | RBM28 | SUFU | WNT7A |
| BAIAP2 | CYSTM1 | GP6 | LOC100130872 | OSBPL5 | RBM6 | SUPT5H | WSB1 |
| BAIAP3 | DAG1 | GPATCH1 | LOC100507472 | OTUD1 | RCBTB2 | SVIL-AS1 | XPO5 |
| BAZ1B | DAZAP1 | GPCPD1 | LOC100507506 | OVCA2 | RCC2 | SYNGAP1 | XRCC1 |
| BBS10 | DBH-AS1 | GPKOW | LOC101409256 | OXSR1 | RCN3 | SYNJ1 | YBX1 |
| BCAT1 | DCLRE1B | GPN2 | LOC101926963 | P2RX7 | RCOR3 | SYNM | YEATS2 |
| BEX1 | DDA1 | GPR160 | LOC101927153 | P3H4 | RFWD3 | SYTL2 | YIPF1 |
| BICD1 | DDAH2 | GPR27 | LOC101927181 | PACERR | RFX3 | SYVN1 | YIPF4 |
| BISPR | DDR2 | GRAP2 | LOC101927550 | PADI2 | RGL4 | TAF1 | ZBTB17 |
| BMS1 | DDX11L10 | GRK5 | LOC101929331 | PALLD | RGP1 | TAF8 | ZBTB7A |
| BPI | DDX19A | GRM2 | LOC102724814 | PANK4 | RHBDF2 | TAOK2 | ZC3H12C |
| BRCAT107 | DDX19B | GRWD1 | LOC200772 | PAOX | RMI1 | TARP | ZC3H13 |
| BRF1 | DDX27 | GSE1 | LOC389765 | PAPOLA | RNF103 | TAS2R41 | ZC3H18 |
| BTBD10 | DDX3X | GTPBP2 | LOC441081 | PAQR7 | RNF138 | TAS2R43 | ZDHHC11 |
| BTBD19 | DDX54 | GTPBP3 | LOC645513 | PAQR9 | RNF146 | TBC1D10B | ZDHHC16 |
| BTN2A3P | DDX55 | GUCY1A3 | LOC729737 | PARK2 | RNF212 | TBC1D15 | ZFHX3 |
| BUD13 | DDX60L | GUCY1B3 | LOC90784 | PARP1 | RNF214 | TBC1D9B | ZFP90 |
| BZRAP1 | DEGS1 | GUSB | LPCAT3 | PC | RNF220 | TBCC | ZMYM3 |
| BZRAP1-AS1 | DEPDC1B | GYG1 | LPL | PCCA | RNFT1 | TCF20 | ZMYND11 |
| C11orf45 | DHCR7 | GYS1 | LPPR2 | PCDHGA11 | RNPC3 | TCHP | ZNF117 |
| C11orf54 | DHRS3 | H2AFX | LRFN1 | PCMTD2 | RPL36AL | TECR | ZNF142 |
| C11orf71 | DHRS7B | HABP4 | LRP1 | PCNT | RPS10P7 | TEF | ZNF175 |
| C15orf52 | DHX16 | HARS | LRRC70 | PCSK6 | RPUSD2 | TENM1 | ZNF230 |
| C15orf54 | DHX38 | HCG27 | LSMEM1 | PCTP | RRBP1 | TERF2 | ZNF282 |
| C18orf32 | DISC1-IT1 | HDAC10 | LTBP3 | PDCD11 | RRS1-AS1 | TERF2IP | ZNF341 |
| C19orf35 | DKC1 | HDLBP | LTBP4 | PDCD6IP | RSRP1 | TFCP2L1 | ZNF408 |
| C1GALT1 | DLG4 | HEBP2 | LUC7L | PDE1B | RUNX1-IT1 | TGFB1 | ZNF500 |
| C1GALT1C1 | DLG5 | HECA | LUZP1 | PDE9A | RUSC2 | TGFB3 | ZNF512B |
| C1orf174 | DNLZ | HERC4 | LYPD2 | PDGFA | S100B | TGFBR1 | ZNF517 |
| C1QTNF6 | DNMT1 | HES6 | MAD1L1 | PDIA3 | SAFB2 | TGM1 | ZNF526 |
| C1R | DOLPP1 | HFE | MAD2L1BP | PDIA4 | SAG | THAP4 | ZNF564 |
| C20orf96 | DOPEY2 | HGS | MAFG | PDK2 | SAP130 | THAP6 | ZNF565 |
| C2CD2L | DPP9 | HHEX | MAN1A2 | PDLIM1 | SAP25 | THAP8 | ZNF57 |
| C2orf42 | DPY19L3 | HINT3 | MAN1C1 | PDZD4 | SAR1B | THBD | ZNF574 |
| C4orf32 | DR1 | HIST1H2AK | MAOA | PEAR1 | SART3 | THBS1 | ZNF609 |
| C6orf120 | DRAM1 | HIST2H2BC | MAP1A | PERP | SAV1 | THSD1 | ZNF610 |
| C6orf25 | DSC2 | HIST2H2BF | MAP2K6 | PES1 | SAXO2 | THTPA | ZNF618 |
| C7orf31 | DTHD1 | HIST2H3D | MAP3K4 | PGM2L1 | SCAF1 | TIMD4 | ZNF654 |
| C7orf60 | DTWD1 | HK3 | MAP3K8 | PHACTR4 | SCAF4 | TIPARP-AS1 | ZNF660 |
| C8orf88 | DTX2 | HLA-DPA1 | MAP7D3 | PHRF1 | SCAMP3 | TJAP1 | ZNF664 |
| C9orf139 | DVL3 | HMGB2 | MAPK8 | PHYHD1 | SCAP | TKFC | ZNF677 |
| CA4 | E4F1 | HMGCL | MAPRE2 | PI4KAP2 | SCCPDH | TLN1 | ZNF74 |
| CABLES2 | EDC4 | HMGN2 | 44260 | PIAS4 | SCN1B | TLR9 | ZNF772 |
| CABP5 | EEF1DP3 | HNRNPAB | MARCKS | PIGO | SDC3 | TMBIM4 | ZNF780A |
| CACNA2D2 | EFCAB12 | HNRNPH1 | MAST2 | PIGR | SDC4 | TMC4 | ZNF788 |
| CACNB1 | EFTUD1 | HNRNPLL | MCEMP1 | PIGT | SDHA | TMCO4 | ZNF790-AS1 |
| CACTIN | EHD3 | HNRNPU-AS1 | MCM5 | PIGX | SEC14L5 | TMED5 | ZNF844 |
| CADM1 | EHMT1 | HNRNPUL1 | MCM8 | PIK3C2B | SEC1P | TMEM104 | ZNF865 |
| CAMP | EHMT2 | HORMAD1 | MCUR1 | PIP5K1C | SEC22B | TMEM156 | ZSCAN2 |
| CAPN11 | EIF2AK4 | | | | | | |

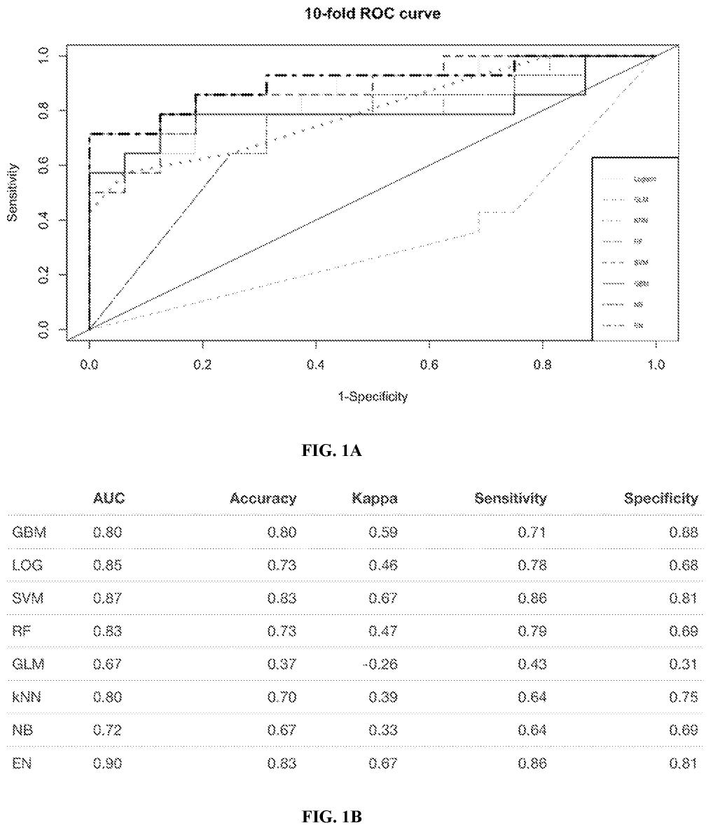

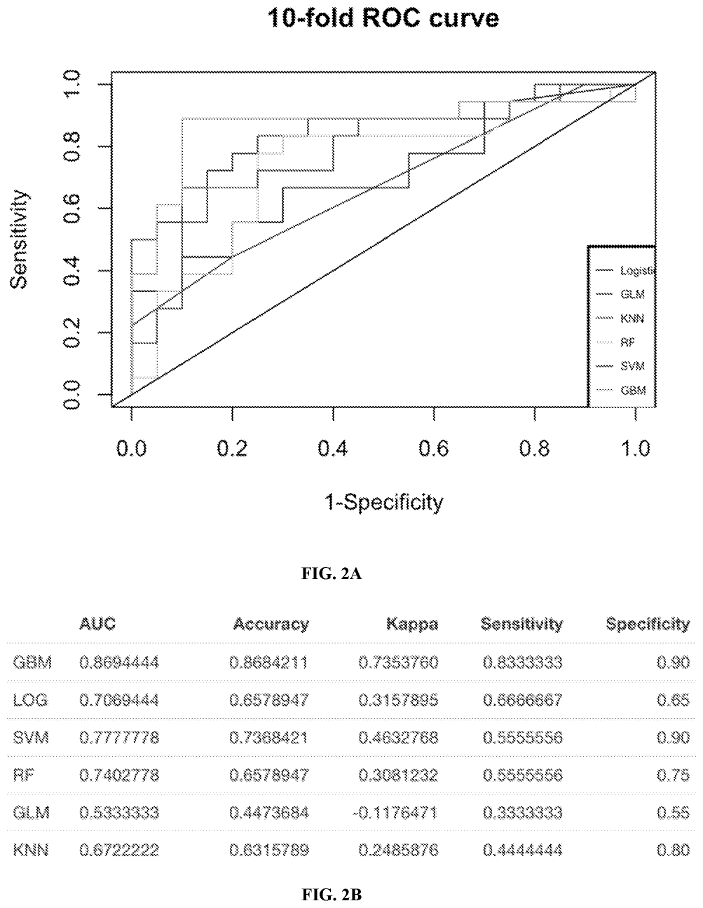

The eight machine learning classifiers were then validated using the 1,178 gene features via a cross validation method. In the cross validation method, the biomarkers dataset was divided into two groups comprising a training set and a validation set. FIGS. 1A-1B show results of a cross validation experiment when 80% of the dataset was considered for training the classifiers while 20% of the dataset was used for validation.

FIG. 1A is a receiver operating characteristic (ROC) plot showing performance of eight machine learning classifiers using a set of 1,178 gene features generated from ribonucleic acid (RNA) sequencing (RNA-Seq) data to distinguish malignant lung nodules versus benign lung nodules. The set of 1,178 genes were differentially expressed in blood samples of patients with malignant lung nodules versus benign lung nodules. The eight machine learning classifiers include LOG, GLM, kNN, RF, SVM, GBM, NB, and EN.

FIG. 1B shows results of exemplary trained machine learning classifier algorithms to analyze RNASeq data using a set of 1,178 gene features to distinguish malignant lung nodules versus benign lung nodules. The corresponding data from the ROC plot of FIG. 1A are tabulated in FIG. 1B. The GBM, SVM, and EN classifiers were the most effective classifiers.

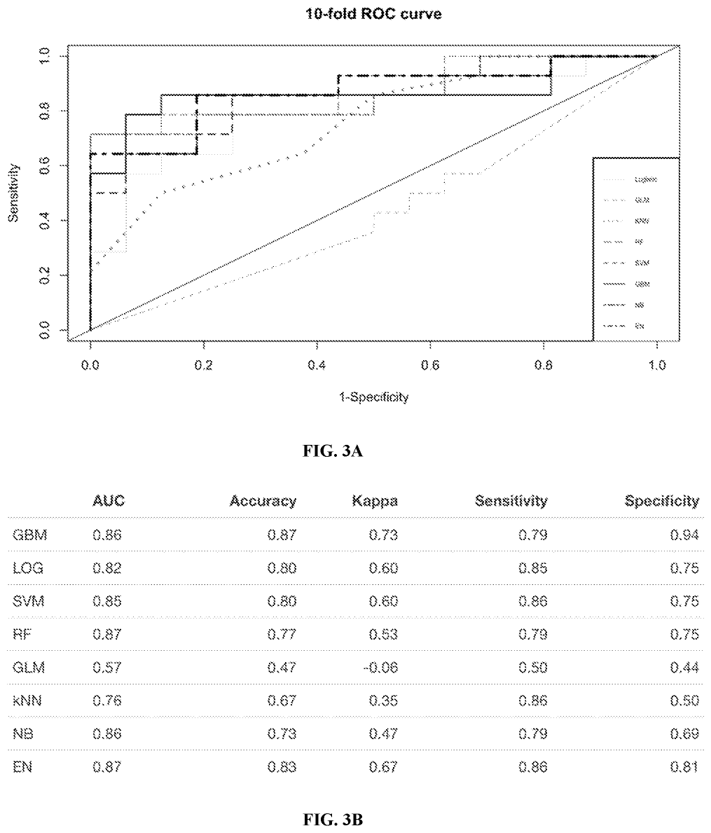

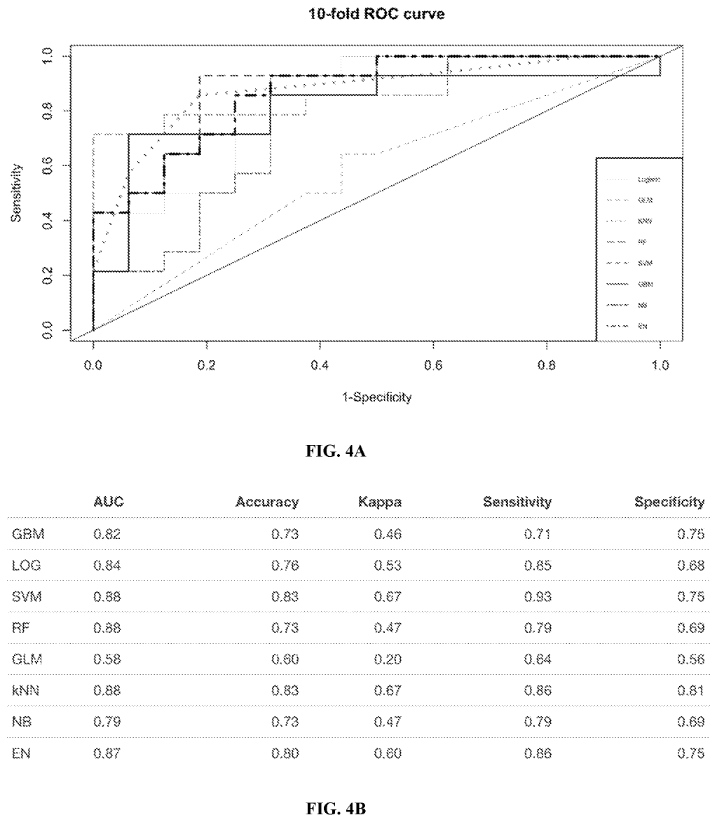

A similar validation was performed using 75% of the dataset for training the classifiers and 25% of the dataset for validation. FIGS. 2A-2B show results of a cross validation experiment when 75% of the dataset was considered for training the classifiers while 25% of the dataset was used for validation.

FIG. 2A is a ROC plot for an optimization of differentially expressed genes to distinguish malignant lung nodules versus benign lung nodules based on an analysis of RNA-Seq data. The six machine learning classifiers include LOG, GLM, kNN, RF, SVM, and GBM. FIG. 2B shows results of exemplary trained machine learning classifier algorithms in an optimization of differentially expressed genes to distinguish malignant lung nodules versus benign lung nodules. The corresponding data from the ROC plot of FIG. 2A are tabulated in FIG. 2B. The GBM, SVM, and kNN classifiers were the most effective classifiers.

In order to obtain a smaller number of features to classify lung nodules, the top 50 predictive genes from the 7 classifiers that accurately predicted lung nodules (FIGS. 1A-1B) were combined. Furthermore, overlapping genes were removed, thereby yielding a gene set of 182 gene features (as shown in Table 1).

TABLE 1
Gene Set of 182 Gene Features

| | | | | | | | |
|---|---|---|---|---|---|---|---|
| ASAP1-IT2 | BEX1 | DPP9 | HP | MTFMT | POM121 | SLC35B3 | TUBA4B |
| UMODL1-AS1 | BMS1 | DSC2 | IFITM3 | NAPB | PPP1R21 | SMG1P5 | UBE2Q1 |
| ABCF1 | BRCAT107 | EEF1DP3 | IFT27 | NAPG | PPP1R3D | SNORA38 | UNC45B |
| ABHD3 | BUD13 | EIF2B3 | KIZ | NBPF10 | PPP2R5A | SPECCIL | UQCC3 |
| ABHD6 | C15orf54 | EIF4ENIF1 | KRBA1 | NCAPD2 | PPP6C | SPPL2A | USF2 |
| ABTB2 | C1GALT1 | EOMES | LASIL | NFE2L1 | PROK2 | SRP68 | USP38 |
| ACLY | CAMP | EXOSC3 | LINC00189 | NR1I2 | PSMD5 | TAF8 | VIL1 |
| ADCY9 | CCNG2 | F8A1 | LINC00925 | NRIR | PTGDS | TAS2R43 | VPS25 |
| ADGRG1 | CD101 | FAM217B | LINC01012 | NT5M | PTGS2 | TECR | WDR20 |
| ADHFE1 | CD177 | FANCG | LINC01347 | NUP210L | RABL6 | TENM1 | WDR45B |
| AGPAT4-IT1 | CDK5R1 | FAS | LOC101927153 | OGFOD3 | RFWD3 | THBS1 | YBX1 |
| AHNAK | CDO1 | FAT4 | LOC101929331 | ORM1 | RNF146 | TIMD4 | YIPF1 |
| AMIGO3 | CHMP4A | FBRS | LPL | OVCA2 | RNF220 | TMEM104 | ZC3H12C |
| ANO6 | CLHC1 | FGFRL1 | LRRC70 | PADI2 | RNFT1 | TMEM156 | ZC3H13 |
| APOBEC3A | CNPY4 | FNBP1 | LSMEM1 | PALLD | RPL36AL | TMEM192 | ZDHHC11 |
| ARG2 | COA4 | FRMPD3 | MADIL1 | PAQR7 | RPS10P7 | TMEM218 | ZDHHC16 |
| ARHGAP21 | COX15 | GINM1 | MAPK8 | PAQR9 | RRBP1 | TMEM65 | ZFHX3 |
| ARHGEF10 | CRCP | GOLGA1 | MED1 | PCCA | SAG | TMPRSS9 | ZFP90 |
| ARRDC3-AS1 | CSF1R | GRK5 | MGST2 | PDLIMI | SAXO2 | TPP1 | ZNF609 |
| ARRDC4 | CYP4F12 | GUSB | MKKS | PHACTR4 | SDHA | TPTEP1 | ZNF772 |
| AZU1 | CYSTM1 | HLA-DPA1 | MKRN3 | PLCB1 | SEPT11 | TRIM26 | ZSCAN2 |
| BAZIB | DDX11L10 | HNRNPU-AS1 | MOGS | PLCH1 | SLC25A14 | TRIM62 | |
| BCAT1 | DNMT1 | HOXB2 | MRC2 | PLVAP | SLC29A3 | TTC38 | |

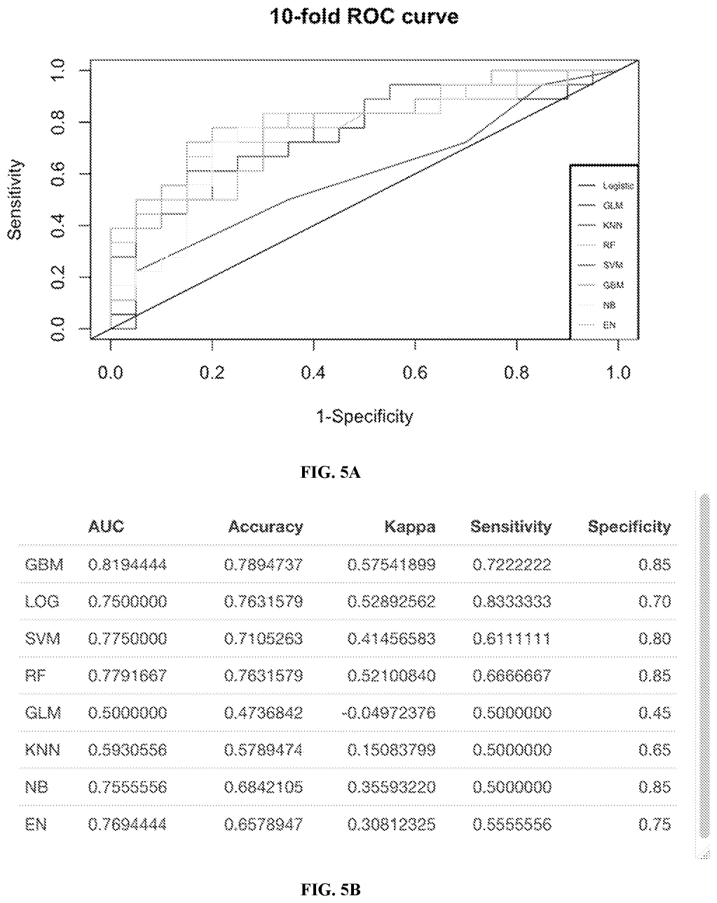

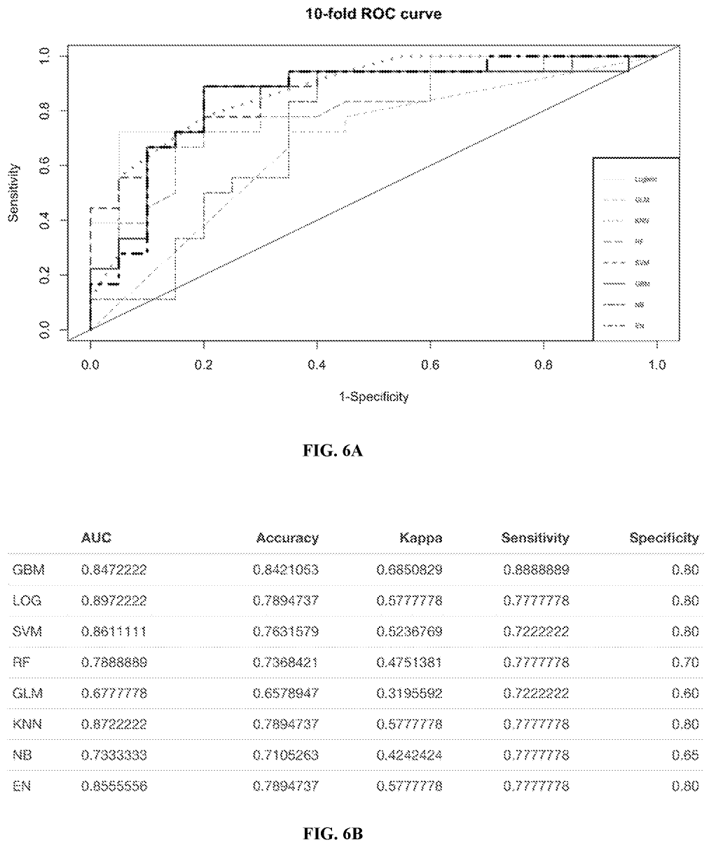

Performance of the classifiers using only the 182 gene features as compared to the 1,178 gene features in predicting lung nodules were examined. Performance results of the seven classifiers using a 10-fold cross validation experiment with 182 gene features are shown in FIGS. 3A-3B.

FIG. 3A is a ROC plot showing performance of seven machine learning classifiers using a set of 182 gene features generated from RNA-Seq data to distinguish malignant lung nodules versus benign lung nodules. The eight machine learning classifiers include LOG, GLM, kNN, RF, SVM, GBM, NB, and EN. The corresponding data from the ROC plot of FIG. 3A are tabulated in FIG. 3B. FIG. 3B shows results of exemplary trained machine learning classifier algorithms to analyze RNASeq data using the set of 182 gene features to distinguish malignant lung nodules versus benign lung nodules.

Each cross validation dataset comprised 80% training data and 20% validation data. The results demonstrated that the 182 gene features effectively distinguished malignant lung nodules versus benign lung nodules. In general, use of the 182 genes was more effective than the entire set of 1,178 genes. Furthermore, the GBM and LOG machine learning classifiers achieved better predictive values when 182 gene features were used, as compared to the entire set of 1,178 gene features. The SVM model achieved a specificity decrease of about 0.05, yet overall performance of the SVM model improved, when the set of 182 gene features was used, as compared to the entire set of 1,178 gene features.

Separately, the entire set of 1,178 genes was examined independently in male subjects and female subjects. The GBM machine learning classifier achieved the best predictive performance for male subject, and the NB machine learning classifier achieved the best predictive performance for female subjects, compared to other classifiers. A gene importance was calculated for each gene feature based on a gene feature from the GBM classifier for males, and the rank for the same gene feature in the NB classifier for females. Genes with a gene importance of >50 were selected for inclusion in a smaller subset, thereby producing a set of 175 gene features from the set of 1,178 gene features initially used to perform the predictions.

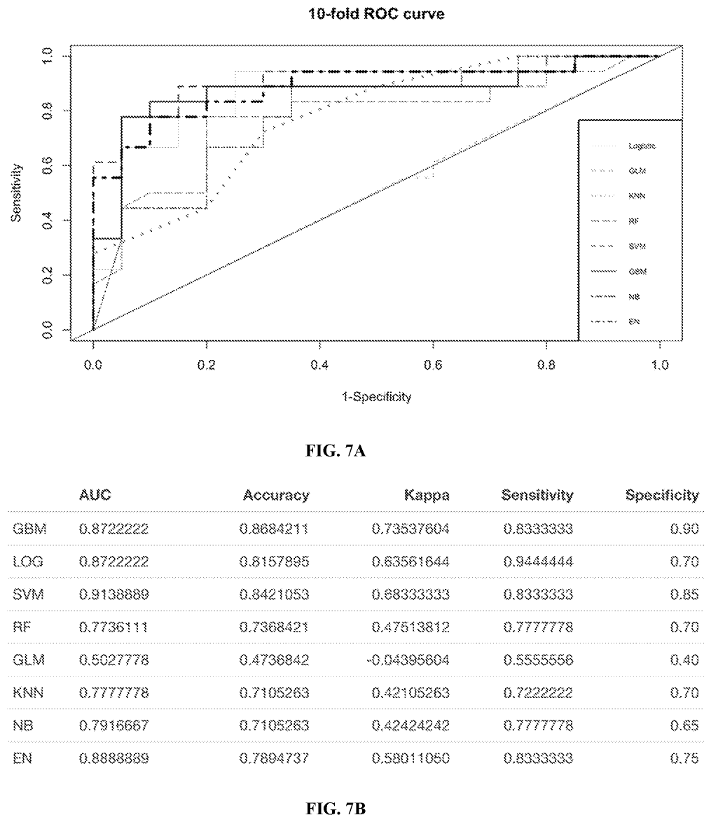

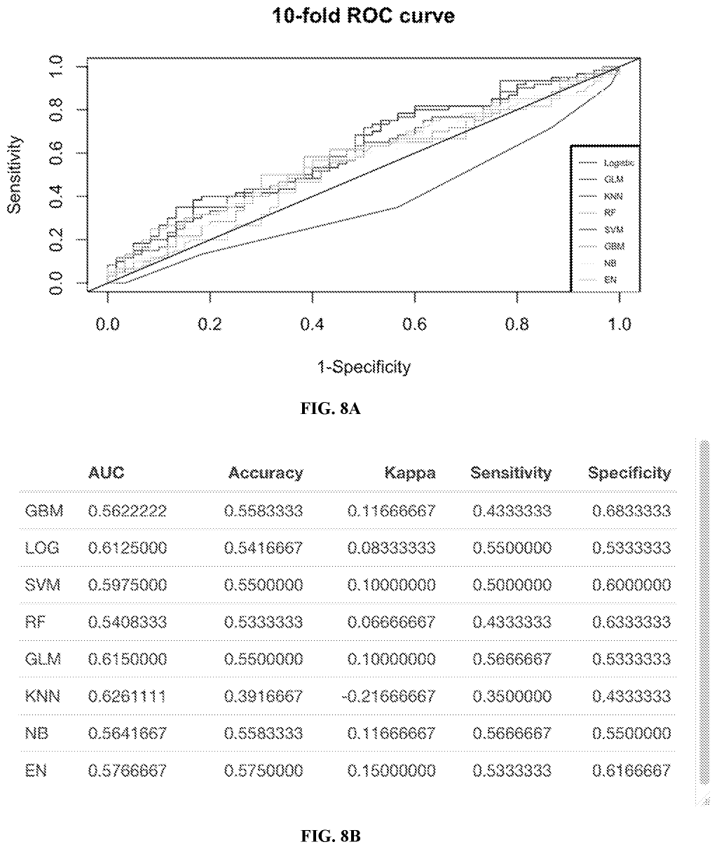

A similar 10-fold cross validation using an 80% training and 20% validation split of the biomarkers dataset was used to examine the effectiveness of the set of 175 gene features using the eight classifiers. FIG. 4A shows the ROC plot of the performance of the classifiers using 175 genes over the entire dataset (males and females). The eight machine learning classifiers include LOG, GLM, kNN, RF, SVM, GBM, NB, and EN. FIG. 4B shows tabulated results of exemplary trained machine learning classifier algorithms corresponding to FIG. 4A.

The corresponding data from the ROC plot of FIG. 4A are tabulated in FIG. 4B. The kNN and EN classifiers achieved better predictive values using the set of 175 gene features as compared to using the set of 182 gene features.

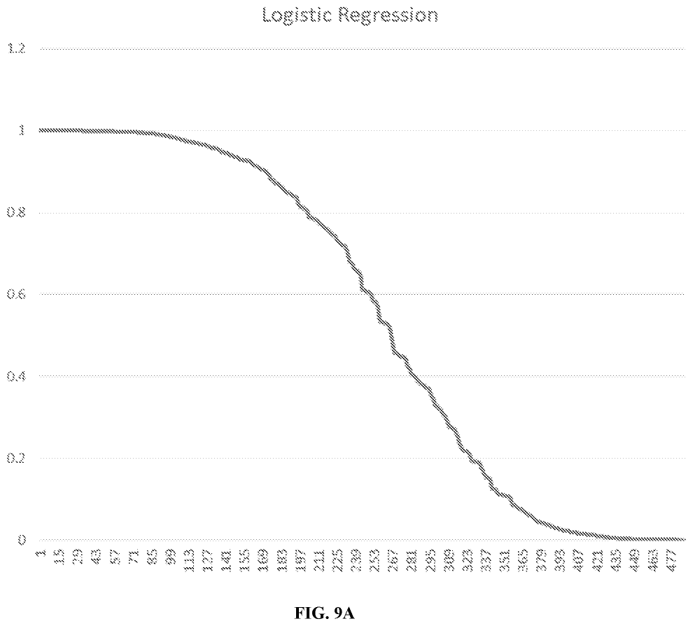

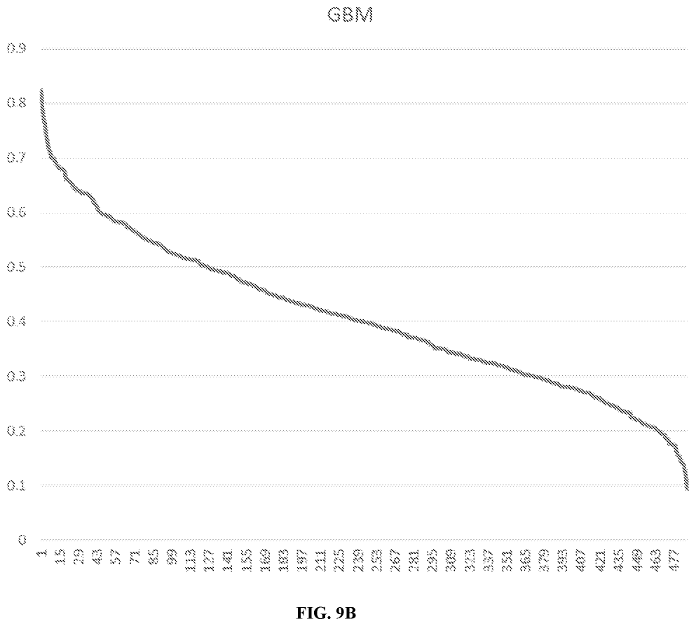

FIG. 5A shows the ROC plot of the eight classifiers' performance using the 175 gene features with a 10-fold validation technique with 80% training and 20% validation split. The eight machine learning classifiers include LOG, GLM, kNN, RF, SVM, GBM, NB, and EN. The corresponding data from the ROC plot of FIG. 5A are tabulated in FIG. 5B. The GBM and SVM classifiers achieved the highest predictive values using the 175 gene features.

TABLE 2
Gene Set of 175 Gene Features

| | | | | | | | |
|---|---|---|---|---|---|---|---|
| ABCF1 | C20orf96 | DTWD1 | GRK5 | MAP2K6 | PARP1 | SART3 | TMEM189 |
| ACLY | C9orf139 | EEF1DP3 | GUSB | MED1 | PDIA3 | SCCPDH | TMEM192 |
| ACTN4 | CCDC94 | EIF2AK4 | HABP4 | MED28 | PDIA4 | SEPT11 | TMEM218 |
| ACTR10 | CD84 | EIF2B5 | HCG27 | MGST2 | PHRF1 | SFSWAP | TMEM56-RWDD3 |
| ADGRG1 | CEBPA | EMC6 | HNRNPAB | MKRN3 | PITRM1 | SLC22A15 | TMEM91 |
| AGPAT4-IT1 | CEP295NL | EMD | HNRNPU-AS1 | MLEC | POLR3D | SLC25A14 | TNFAIP8L1 |
| AHNAK | CFAP58-AS1 | ENTPD6 | HOXB2 | MOGS | PPP1R21 | SLC35B3 | TSPAN33 |
| AKRIC1 | CHCHD10 | FAS | IL18 | MSMO1 | PSMD5 | SMAD7 | U2AF2 |
| ANKRD17 | CHD3 | FGD2 | INO80 | MTA2 | PTBP1 | SMARCD3 | UBA1 |
| ANO6 | CHD8 | FLJ37453 | KIAA0100 | MTFMT | PTGFR | SOCS7 | UCP3 |
| ARHGEF1 | CLHC1 | FLT3 | KIF3B | MXD3 | PTPN18 | SPECCIL | UHMK1 |
| ARRDC3-AS1 | COA4 | FNBP1 | LAIR1 | MYLK | PYGB | SPN | VARS |
| ATAD3B | COMMD3 | GANAB | LASIL | MYOF | RABL6 | SRP68 | VPS25 |
| AVPRIA | CXCL1 | GDI1 | LETM1 | NAPB | RASA3 | STT3A | WDR20 |
| BAHD1 | CYSTM1 | GFOD1 | LINC00493 | NCAPD2 | RCC2 | SUPT5H | YEATS2 |
| BAZIB | DAZAP1 | GIGYF2 | LINC00671 | NCK2 | RFWD3 | SYNM | ZC3H12C |
| BCAT1 | DDX54 | GINMI | LOC100049716 | NFE2L1 | RMI1 | TAF8 | ZC3H13 |
| BEX1 | DHX16 | GLG1 | LOC101927153 | NMT1 | RNFT1 | TAS2R43 | ZDHHC16 |
| BICD1 | DHX38 | GOLGA2 | LOC101929331 | OBFC1 | RRBP1 | TCF20 | ZNF117 |
| BMS1 | DKC1 | GOLGA3 | LPL | OGFOD3 | RUNX1-IT1 | TCHP | ZNF230 |
| C11orf71 | DNMT1 | GPKOW | LUZPI | ORAI3 | SAFB2 | THBS1 | ZNF772 |
| C15orf54 | DSC2 | GRAP2 | MAPIA | OVCA2 | SAG | TLN1 | |

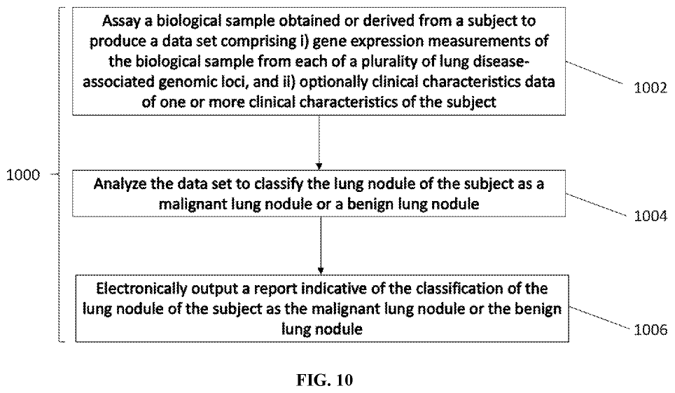

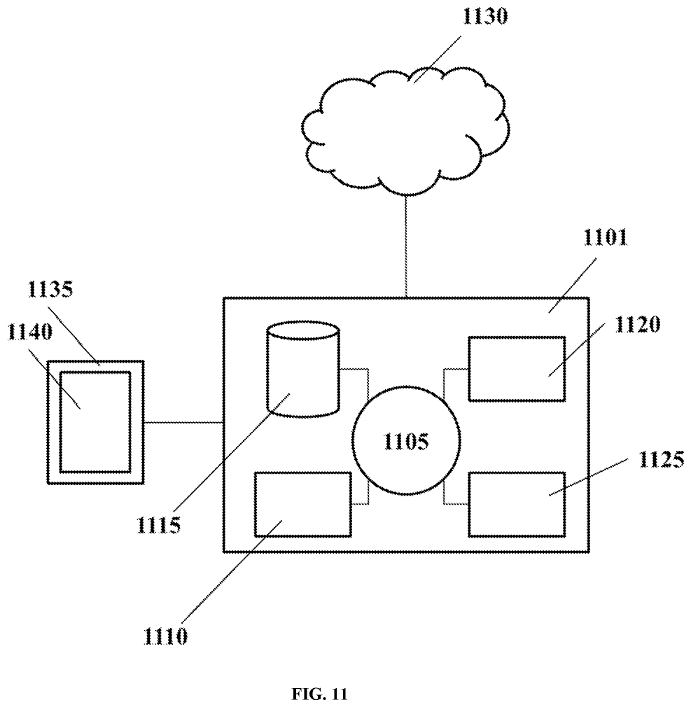

The set of 175 gene features and the set of 182 gene features had a total of shared 62 gene features which overlapped between the two sets. The 62 gene features were examined for their effectiveness in predicting lung nodules using the biomarkers dataset. 10-fold cross validation with training to validation split of 75% and 25% was used. 6B shows tabulated results of exemplary trained machine learning classifier algorithms corresponding to FIG. 6A. FIG. 6A is a ROC plot showing performance of machine learning classifiers using a set of the 62 gene features generated from RNA-Seq data to distinguish malignant lung nodules versus benign lung nodules. The eight machine learning classifiers include LOG, GLM, kNN, RF, SVM, GBM, NB, and EN. The set of 62 gene features achieved high predictive value across all eight classifiers.

TABLE 3
Gene Set of 62 Gene Features Shared Between Tables 1 and 2

| | | | | | | | |
|---|---|---|---|---|---|---|---|
| ABCF1 | BCAT1 | DSC2 | HOXB2 | MOGS | PSMD5 | SLC35B3 | VPS25 |
| ACLY | BEX1 | EEF1DP3 | LASIL | MTFMT | RABL6 | SPECCIL | WDR20 |
| ADGRG1 | BMS1 | FAS | LOC101927153 | NAPB | RFWD3 | SRP68 | ZC3H12C |
| AGPAT4-IT1 | C15orf54 | FNBP1 | LOC101929331 | NCAPD2 | RNFT1 | TAF8 | ZC3H13 |
| AHNAK | CLHC1 | GINM1 | LPL | NFE2L1 | RRBP1 | TAS2R43 | ZDHHC16 |
| ANO6 | COA4 | GRK5 | MED1 | OGFOD3 | SAG | THBS1 | ZNF772 |
| ARRDC3-AS1 | CYSTM1 | GUSB | MGST2 | OVCA2 | SEPT11 | TMEM192 | |
| BAZIB | DNMT1 | HNRNPU-AS1 | MKRN3 | PPP1R21 | SLC25A14 | TMEM218 | |

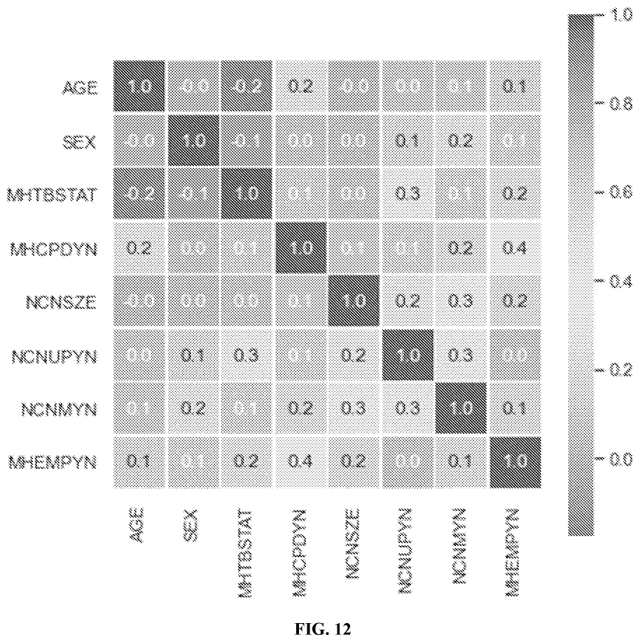

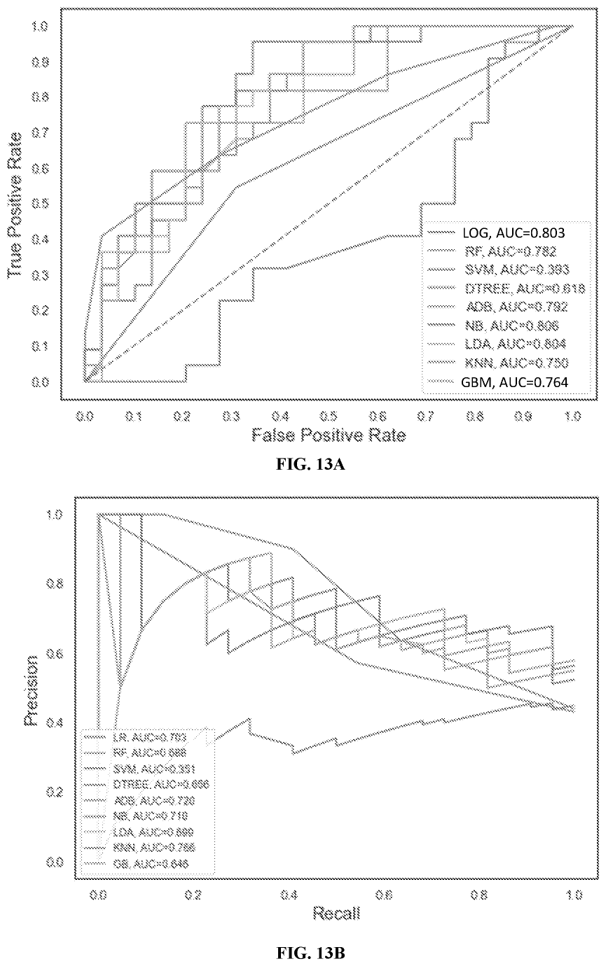

Separately, the set of 182 gene features and the set of 175 gene features were combined and overlapping genes were removed to produce a set of 295 gene features. This set of 295 gene features was tested using the biomarkers database to examine the effectiveness in classifying lung cancers. Classifiers were tested using the 295 gene features using a 10-fold cross validation technique with a 75% to 25% split to generate training and validation datasets. FIG. 7A is a ROC plot showing performance of machine learning classifiers using a set of 295 gene features generated from RNA-Seq data to distinguish malignant lung nodules versus benign lung nodules. The eight machine learning classifiers include LOG, GLM, kNN, RF, SVM, GBM, NB, and EN.

FIG. 7B shows tabulated results of exemplary trained machine learning classifier algorithms corresponding to FIG. 7A. All classifiers except GLM achieved high predictive values in classifying lung nodules using the biomarkers dataset.

TABLE 4
Gene Set of 295 Gene Features Included in Tables 1 and 2

| | | | | | | | |
|---|---|---|---|---|---|---|---|
| ABCF1 | CIGALT1 | DTWD1 | HCG27 | MKKS | PHRF1 | SEPT11 | TPP1 |
| ABHD3 | C20orf96 | EEF1DP3 | HLA-DPA1 | MKRN3 | PITRM1 | SFSWAP | TPTEP1 |
| ABHD6 | C9orf139 | EIF2AK4 | HNRNPAB | MLEC | PLCB1 | SLC22A15 | TRIM26 |
| ABTB2 | CAMP | EIF2B3 | HNRNPU-AS1 | MOGS | PLCH1 | SLC25A14 | TRIM62 |
| ACLY | CCDC94 | EIF2B5 | HOXB2 | MRC2 | PLVAP | SLC29A3 | TSPAN33 |
| ACTN4 | CCNG2 | EIF4ENIF1 | HP | MSMO1 | POLR3D | SLC35B3 | TTC38 |
| ACTR10 | CD101 | EMC6 | IFITM3 | MTA2 | POM121 | SMAD7 | TUBA4B |
| ADCY9 | CD177 | EMD | IFT27 | MTFMT | PPP1R21 | SMARCD3 | U2AF2 |
| ADGRG1 | CD84 | ENTPD6 | IL18 | MXD3 | PPP1R3D | SMG1P5 | UBA1 |
| ADHFE1 | CDK5R1 | EOMES | INO80 | MYLK | PPP2R5A | SNORA38 | UBE2Q1 |
| AGPAT4-IT1 | CDO1 | EXOSC3 | KIAA0100 | MYOF | PPP6C | SOCS7 | UCP3 |
| AHNAK | CEBPA | F8A1 | KIF3B | NAPB | PROK2 | SPECCIL | UHMK1 |
| AKRIC1 | CEP295NL | FAM217B | KIZ | NAPG | PSMD5 | SPN | UMODL1-AS1 |
| AMIGO3 | CFAP58-AS1 | FANCG | KRBA1 | NBPF10 | PTBP1 | SPPL2A | UNC45B |
| ANKRD17 | CHCHD10 | FAS | LAIR1 | NCAPD2 | PTGDS | SRP68 | UQCC3 |
| ANO6 | CHD3 | FAT4 | LASIL | NCK2 | PTGFR | STT3A | USF2 |
| APOBEC3A | CHD8 | FBRS | LETM1 | NFE2L1 | PTGS2 | SUPT5H | USP38 |
| ARG2 | CHMP4A | FGD2 | LINC00189 | NMT1 | PTPN18 | SYNM | VARS |
| ARHGAP21 | CLHC1 | FGFRL1 | LINC00493 | NR112 | PYGB | TAF8 | VIL1 |
| ARHGEF1 | CNPY4 | FLJ37453 | LINC00671 | NRIR | RABL6 | TAS2R43 | VPS25 |
| ARHGEF10 | COA4 | FLT3 | LINC00925 | NT5M | RASA3 | TCF20 | WDR20 |
| ARRDC3-AS1 | COMMD3 | FNBP1 | LINC01012 | NUP210L | RCC2 | TCHP | WDR45B |
| ARRDC4 | COX15 | FRMPD3 | LINC01347 | OBFC1 | RFWD3 | TECR | YBX1 |
| ASAP1-IT2 | CRCP | GANAB | LOC100049716 | OGFOD3 | RMI1 | TENM1 | YEATS2 |
| ATAD3B | CSFIR | GDI1 | LOC101927153 | ORAI3 | RNF146 | THBS1 | YIPF1 |
| AVPRIA | CXCL1 | GFOD1 | LOC101929331 | ORM1 | RNF220 | TIMD4 | ZC3H12C |
| AZU1 | CYP4F12 | GIGYF2 | LPL | OVCA2 | RNFT1 | TLN1 | ZC3H13 |
| BAHD1 | CYSTM1 | GINM1 | LRRC70 | PADI2 | RPL36AL | TMEM104 | ZDHHC11 |
| BAZIB | DAZAP1 | GLG1 | LSMEM1 | PALLD | RPS10P7 | TMEM156 | ZDHHC16 |
| BCAT1 | DDX11L10 | GOLGA1 | LUZP1 | PAQR7 | RRBP1 | TMEM189 | ZFHX3 |
| BEX1 | DDX54 | GOLGA2 | MAD1L1 | PAQR9 | RUNX1-IT1 | TMEM192 | ZFP90 |
| BICD1 | DHX16 | GOLGA3 | MAPIA | PARP1 | SAFB2 | TMEM218 | ZNF117 |
| BMS1 | DHX38 | GPKOW | MAP2K6 | PCCA | SAG | TMEM56-RWDD3 | ZNF230 |
| BRCAT107 | DKC1 | GRAP2 | MAPK8 | PDIA3 | SART3 | TMEM65 | ZNF609 |
| BUD13 | DNMT1 | GRK5 | MED1 | PDIA4 | SAXO2 | TMEM91 | ZNF772 |
| C11orf71 | DPP9 | GUSB | MED28 | PDLIM1 | SCCPDH | TMPRSS9 | ZSCAN2 |
| C15orf54 | DSC2 | HABP4 | MGST2 | PHACTR4 | SDHA | TNFAIP8L1 | |

Results demonstrated that machine learning classifiers performed well to distinguish malignant lung nodules from benign lung nodules. Feature selection was performed to reduce the set of features from 1,178 genes to one of (i) a set of 295 genes, (ii) a set of 182 genes, (iii) a set of 175 genes, or (iv) a set of 62 genes, which achieved positive results in distinguishing malignant lung nodules from benign lung nodules. In the following examples, larger datasets were investigated to compensate for heterogeneity in clinical data.

The top 50 predictors from seven classifiers were selected and after removing overlapping genes, a set of 142 gene features (Table 5) were obtained. The seven classifiers included the eight classifiers other than the GLM. Gene expression data for the set of 142 gene features were obtained using RNA-Seq. All eight classifiers were trained and validated using the set of 142 gene features over the biomarkers dataset using a 10-fold cross validation technique with 80% to 20% training and validation data split.

TABLE 5
Gene Set of the 142 gene features.

| | | | | | | |
|---|---|---|---|---|---|---|
| ABCF1 | CEP250 | GUSB | MIR22HG | PLCB1 | SAV1 | TSPAN33 |
| ABHD3 | CHMP4A | HDAC3 | MIR3939 | PLCH1 | SCAMP3 | UCP2 |
| ABHD6 | CLHC1 | HERC4 | MKKS | PLVAP | SDHA | UQCC3 |
| ACLY | CNPY4 | HLA-DPA1 | MKRN3 | POLR3D | SEPT11 | USF2 |
| ADCY9 | COA4 | HMGCL | MRC2 | POM121 | SLC25A14 | USP38 |
| AHNAK | COL6A3 | HNRNPH1 | MTFMT | PPP1R21 | SLC35B3 | VIL1 |
| ANO6 | COX15 | HNRNPU-AS1 | NAPB | PPP1R3D | SMG1P5 | VPS26A |
| AP3D1 | CRCP | HOXB2 | NCAPD2 | PPP2R5A | SNORA25 | VPS37C |
| ARHGAP21 | CTSA | IFITM3 | NFE2L1 | PPP6C | SPECC1L | VTA1 |
| ASAP1-IT2 | CYSTM1 | KIZ | NOMO2 | PROK2 | SRP68 | WDR20 |
| BAZ1B | DNMT1 | LINC00944 | NPL | PSMC4 | TAF8 | WDR45B |
| BCAT1 | EEF1DP3 | LINC01126 | NUP210L | PSMD5 | TDRD9 | YIPF1 |
| BRCAT107 | EIF2B3 | LOC100130093 | OGFOD3 | PTGS2 | TECR | ZBTB17 |
| BUD13 | EXOSC3 | LOC101929331 | OVCA2 | PTX3 | TENM1 | ZC3H12C |
| C15orf54 | F8A1 | LOC389765 | PALLD | RABL6 | TGFB1 | ZDHHC16 |
| C6orf120 | FAM161B | LPL | PAQR7 | RFWD3 | TMEM156 | ZFP90 |
| CAMP | FAM217B | LYPD2 | PCCA | RNF220 | TMEM218 | ZNF564 |
| CCNG2 | FAS | MAD1L1 | PCSK6 | RNPC3 | TMEM65 | ZNF609 |
| CCNL1 | FNBP1 | MED1 | PDGFA | RPL36AL | TMEM8A | ZNF772 |
| CD101 | GALNT14 | MGST2 | PKD1P6 | RRBP1 | TRMT1L | ZSCAN2 |
| CDK5R1 | GOLGA1 | | | | | |

### Example 2: Machine Learning Classification of Ampli-Sec Data

A larger dataset from 604 subjects was assembled to examine the effectiveness of the set of 175 gene features in distinguishing malignant versus benign lung nodules. Gene expression measurements of whole blood samples from the subjects were analyzed using Ampli-Seq technique. The training dataset was obtained using Ampli-Seq targeting the 175 genes determined previously. The training dataset comprised 301 lung nodule samples that were known to be benign and 303 samples that were diagnosed as malignant. Normalized Ampli-Seq read counts (RPM) of the 175 genes were provided as input data to the classifiers.

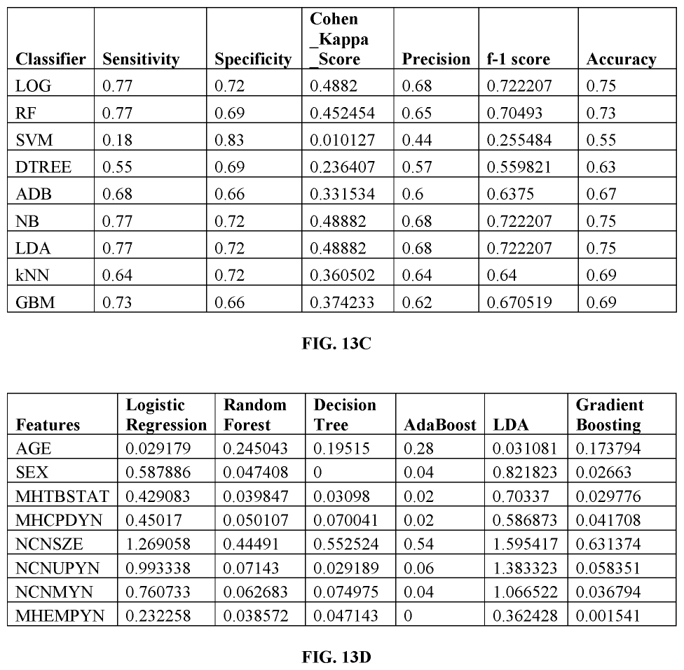

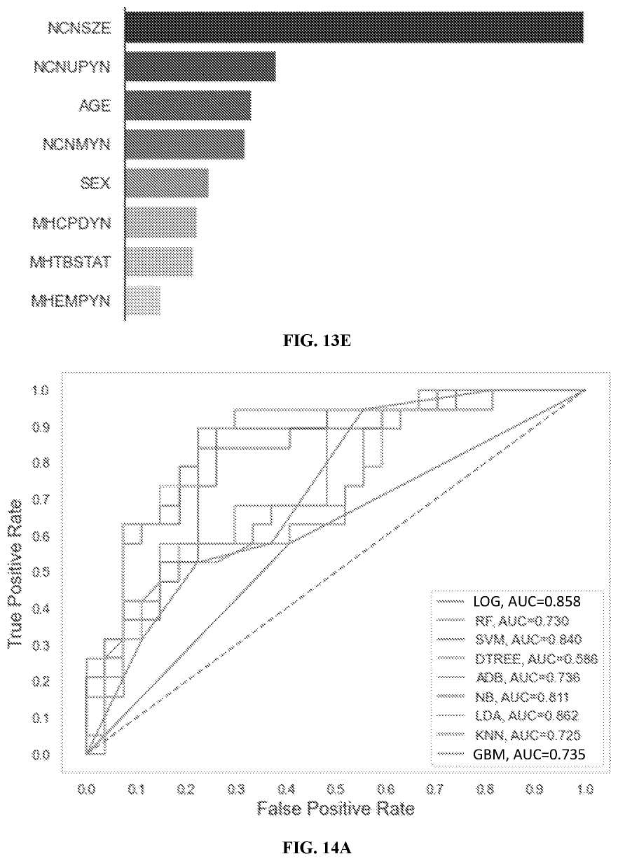

Results of the eight classifiers in a 10-fold validation using a data split of 80% training data to 20% validation data is shown in FIGS. 8A-8B. FIG. 8A is a ROC plot showing performance of machine learning classifiers using a set of 175 gene features generated from Ampli-Seq data to distinguish malignant lung nodules versus benign lung nodules. The eight machine learning classifiers include LOG, GLM, kNN, RF, SVM, GBM, NB, and EN. FIG. 8B shows tabulated results of exemplary trained machine learning classifier algorithms corresponding to FIG. 8A. A similar 10-fold validation was performed using a training to validation data split of 75% to 25%.

### Example 3: Machine Learning Classification and Validation Using Ampli-Sec Data

The performance of the machine learning classifiers of Example 2 was validated using a dataset of lung nodule samples from 487 subjects. The validation dataset was obtained using Ampli-Seq targeting the set of 175 genes. The validation dataset comprised 142 lung nodule samples that were diagnosed as being malignant.

Normalized Ampli-Seq read counts (RPM) of the set of 175 genes were provided as input data to the classifiers. The best performing classifier using the set of 175 gene features (LOG) and the set of 85 gene features (GBM) were compared on the validation dataset. Data from the validation dataset was not used to train the classifiers.

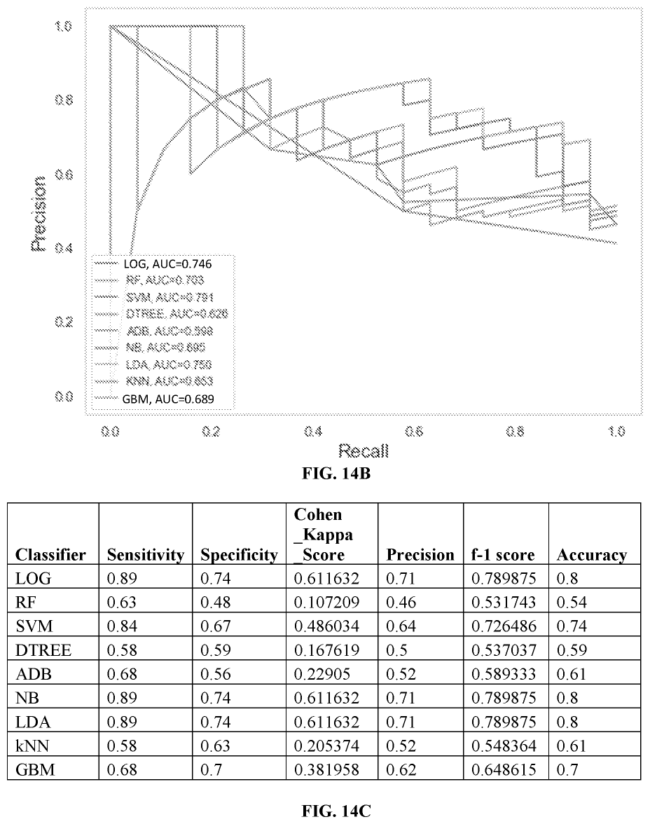

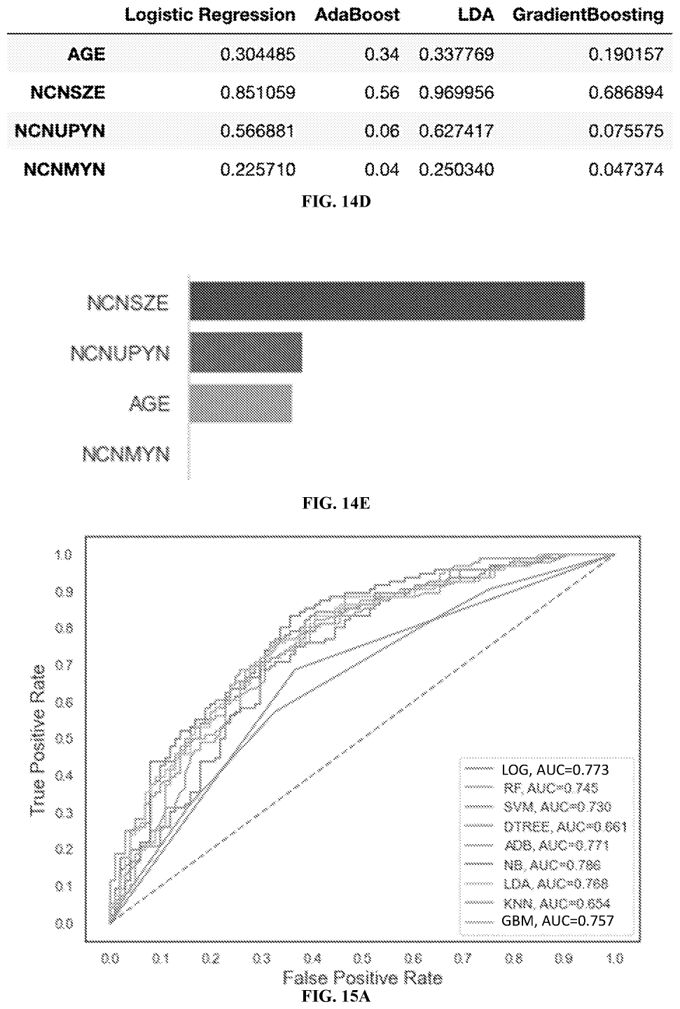

FIG. 9A is a cumulative fraction of lung nodules predicted by a logistic regression classifier using a set of 175 gene features. FIG. 9B is a cumulative fraction of lung nodules predicted by a gradient boosting classifier using the set of 175 gene features.

The cumulative fraction of malignant lung nodules predicted by the LOG model using the set of 175 features (FIG. 9A) showed overfitting when compared to the GBM using the set of 85 features (FIG. 9B). The LOG classifier identified 266 patients with malignant lung nodules from the total of 487 patients (FIG. 9A). Meanwhile, using the subset of 85 genes, the GBM classifier identified 127 out of 142 patients with malignant lung nodules versus benign lung nodules.

### Example 4: Machine Learning Classification Using Clinical Characteristics Data

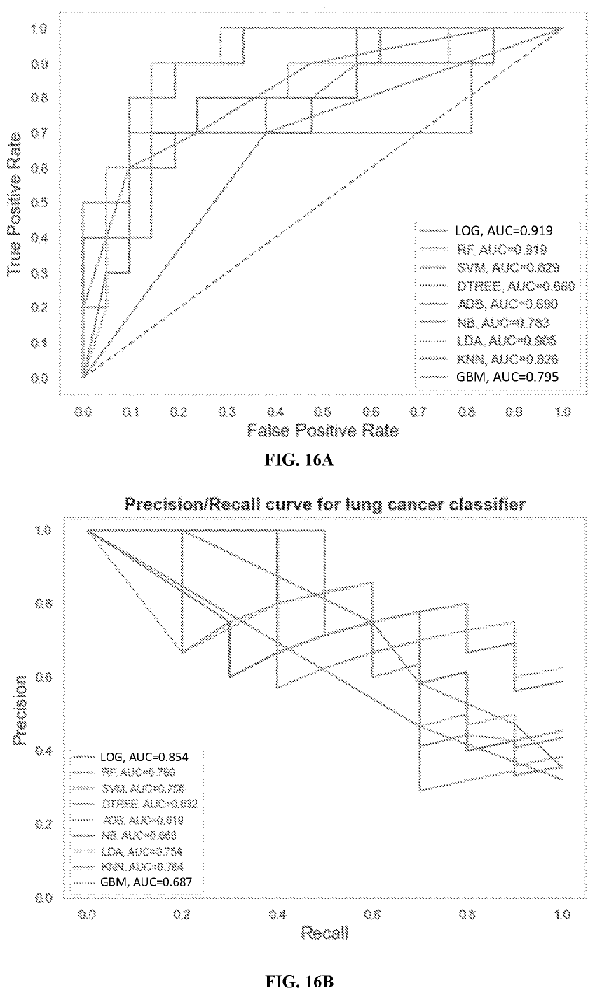

A biomarker dataset obtained from 152 subjects was analyzed. Among those, 80 subjects had a diagnosis of a benign lung nodule, and 72 subject had a diagnosis of a malignant lung nodule. A set of 8 clinical characteristics features (Table 6) were examined for their effectiveness in predicting lung nodules using the biomarkers dataset. FIG. 12 shows the correlation plot of the 8 clinical characteristics features (Table 6).

TABLE 6
Clinical Characteristics
AGE (age of the subject)
SEX (sex of the subject)
NCNSZE (nodule size)
NCNUPYN (nodule in the upper lobe; Yes/No)
MHTBSTAT (Smoking status; Past/Current)
MHCPDYN (Chronic obstructive pulmonary
disease; Yes/No)
NCNMYN (Nodule Spiculated; Yes/No )
MHEMPYN (Emphysemal; Yes/No)

Eight machine learning classifiers including Logistic regression model (LOG), Random forest (RF), Support vector machines (SVM), Decision tree learning (DTREE), Adaptive boosting (ADB), Naïve Bayes (NB), Linear discriminant analysis (LDA), k-nearest neighbors (kNN), and Gradient boosting machines (GBM), were trained to distinguish malignant lung nodules versus benign lung nodules based on clinical characteristics data of the 8 clinical characteristics features (Table 6).

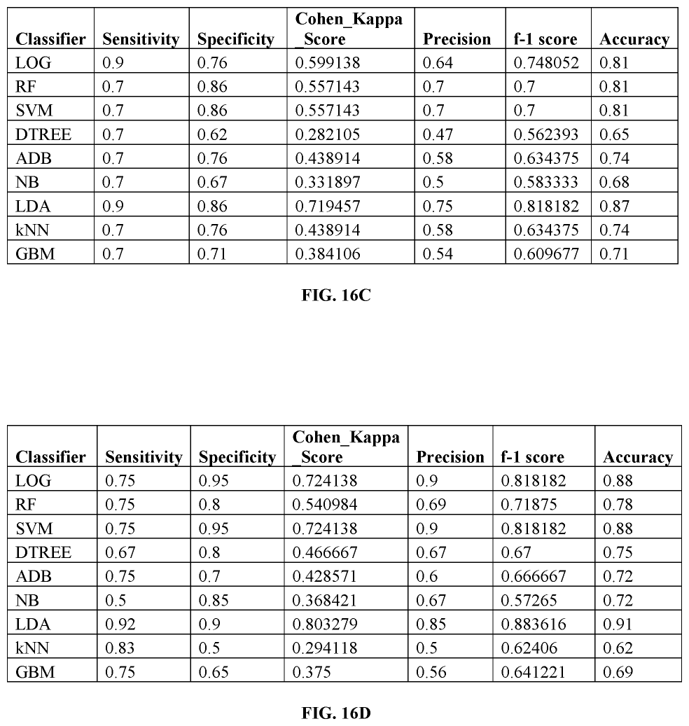

FIG. 13A shows ROC plots showing performance of the 9 machine learning classifiers using clinical characteristics data of the 8 clinical characteristics features (Table 6), to distinguish malignant lung nodules versus benign lung nodules. 10-fold cross validation using an 80% training and 20% validation split of the biomarkers dataset was used. AUC of the ROC plots for the 9 machine learning classifiers LOG, RF, SVM, DTREE, ADB, NB, LDA, kNN, and GBM, are 0.803, 0.782, 0.393, 0.618, 0.792, 0.806, 0.804, 0.750 and 0.764 respectively. FIG. 13B shows Precision/Recall curve of the 9 machine learning classifiers using clinical characteristics data of the 8 clinical characteristics features, to distinguish malignant lung nodules versus benign lung nodules. AUC of the Precision/Recall curve for the 9 machine learning classifiers LOG, RF, SVM, DTREE, ADB, NB, LDA, kNN, and GBM, are 0.703, 0.688, 0.351, 0.656, 0.720, 0.710, 0.699, 0.766 and 0.646 respectively. FIG. 13C presents the tabulated results of the 9 machine learning classifiers corresponding to FIG. 13A. FIG. 13D presents feature importance of the 8 clinical characteristics features for the 9 machine learning classifiers. FIG. 13E shows feature importance of the 8 clinical characteristics features for all the 9 classifiers. As can be seen from FIGS. 13D and E, the three top most contributors or predictors or features were NCNSZE (nodule size), NCNUPYN (nodule in the upper lobe), and AGE, with the fourth being NCNMYN (Nodule Spiculated).

Next, the effectiveness of the top 4 features as determined above, e.g. NCNSZE (nodule size), NCNUPYN (nodule in the upper lobe), AGE, and NCNMYN (Nodule Spiculated), were examined using the eight classifiers.

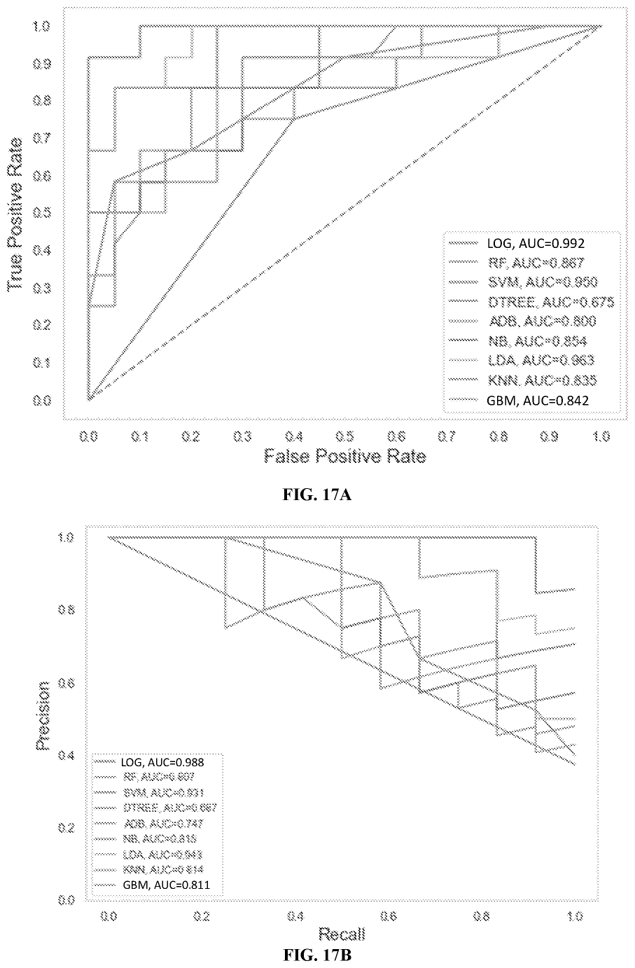

FIG. 14A shows ROC plots showing performance of the 9 machine learning classifiers using clinical characteristics data of the 4 clinical characteristics features, NCNSZE, NCNUPYN, AGE, and NCNMYN to distinguish malignant lung nodules versus benign lung nodules. 10-fold cross validation using an 80% training and 20% validation split of the biomarkers dataset was used. AUC of the ROC plots for the 9 machine learning classifiers LOG, RF, SVM, DTREE, ADB, NB, LDA, kNN, and GBM, are 0.858, 0.730, 0.840, 0.586, 0.736, 0.811, 0.862, 0.725 and 0.735 respectively. FIG. 14B shows Precision/Recall curve of the 9 machine learning classifiers using clinical characteristics data of the 4 clinical characteristics features, NCNSZE, NCNUPYN, AGE, and NCNMYN, to distinguish malignant lung nodules versus benign lung nodules. AUC of the Precision/Recall curve for the 9 machine learning classifiers LOG, RF, SVM, DTREE, ADB, NB, LDA, kNN, and GBM, are 0.746, 0.703, 0.791, 0.626, 0.598, 0.695, 0.750, 0.653 and 0.689 respectively. FIG. 14C presents the tabulated results of the 9 machine learning classifiers corresponding to FIG. 14A. FIG. 14D presents feature importance of the 4 clinical characteristics features for the 9 machine learning classifiers. FIG. 14E shows feature importance of the 4 clinical characteristics features for all the 9 classifiers. As can be seen from FIGS. 13A and 14A, performance of the classifiers when used top 4 predictors (NCNSZE, NCNUPYN, AGE, and NCNMYN) shows better performances than all 8 predictors (Table 6).

A larger dataset from 604 subjects was assembled to examine the effectiveness of the clinical features in distinguishing malignant versus benign lung nodules. Among those, 301 of the samples in the biomarker dataset had a diagnosis of a benign lung nodule, and 303 samples had a diagnosis of a malignant lung nodule. A set of 9 clinical characteristics features (clinical characteristics in Table 6, and cancer history—Y/N)) were examined for their effectiveness in predicting lung nodules using the larger dataset.

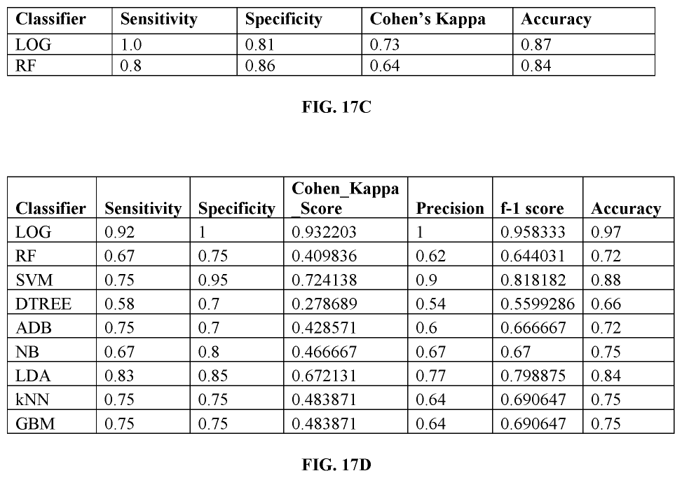

FIG. 15A shows ROC plots showing performance of the 9 machine learning classifiers using clinical characteristics data of the 9 clinical characteristics features, to distinguish malignant lung nodules versus benign lung nodules. 10-fold cross validation using an 80% training and 20% validation split of the larger dataset was used. AUC of the ROC plots for the 9 machine learning classifiers LOG, RF, SVM, DTREE, ADB, NB, LDA, kNN, and GBM, are 0.773, 0.745, 0.730, 0.661, 0.771, 0.786, 0.768, 0.654 and 0.757 respectively. FIG. 15B shows Precision/Recall curve of the 9 machine learning classifiers using clinical characteristics data of the 9 clinical characteristics features, to distinguish malignant lung nodules versus benign lung nodules. AUC of the Precision/Recall curve for the 9 machine learning classifiers LOG, RF, SVM, DTREE, ADB, NB, LDA, kNN, and GBM, are 0.747, 0.690, 0.673, 0.740, 0.759, 0.746, 0.743, 0.633 and 0.707 respectively. FIG. 15C shows the tabulated results of the 9 machine learning classifiers corresponding to FIG. 15A. FIG. 15D shows feature importance of the 9 clinical characteristics features for the 9 machine learning classifiers. FIG. 15E shows feature importance of the 9 clinical characteristics features for all the 9 models. As can be seen from FIGS. 15D and E, the three top most contributors or predictors or features were NCNSZE (nodule size), NCNUPYN (nodule in the upper lobe), and AGE.

### Example 5: Machine Learning Classification Using Gene Expression Data and Clinical Characteristics Data

Based on the results, obtained in the above examples, a combination of a set of 142 gene features (Table 5), and a set of 3 clinical characteristics features were examined for their effectiveness in predicting lung nodules. The 142 gene features were selected based on results of Example 1. The 3 clinical characteristics features, NCNSZE (nodule size), NCNUPYN (nodule in the upper lobe), and AGE, were selected based on the results of Example 4. Gene expression measurements were from whole blood samples of the subjects. A combined biomarker dataset comprising samples from the 152 subjects was analyzed. Among those, 80 subjects had a diagnosis of a benign lung nodule, and 72 subjects had a diagnosis of a malignant lung nodule.

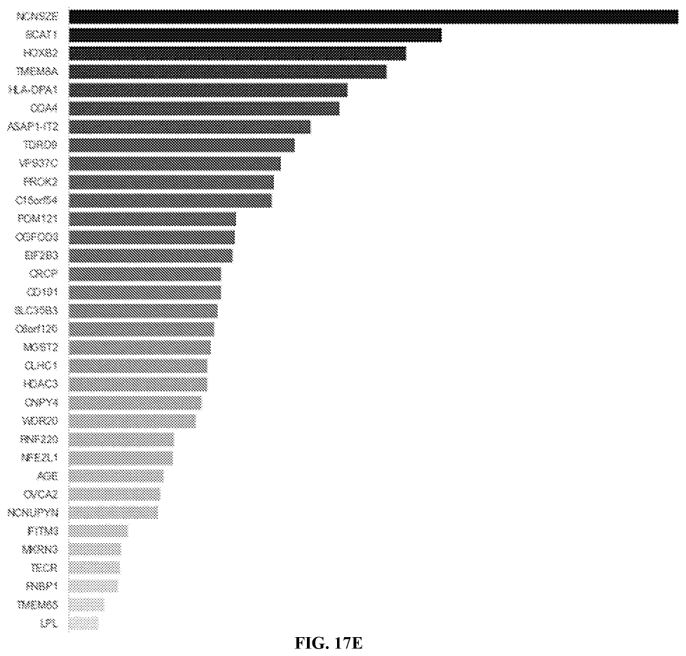

FIG. 16A shows ROC plots showing performance of the 9 machine learning classifiers using gene expression data of the 142 gene features, and clinical characteristics data of the 3 clinical characteristics to distinguish malignant lung nodules versus benign lung nodules. 10-fold cross validation using an 80% training and 20% validation split of the combined dataset was used. AUC of the ROC plots for the 9 machine learning classifiers LOG, RF, SVM, DTREE, ADB, NB, LDA, kNN, and GBM, are 0.919, 0.819, 0.829, 0.660, 0.690, 0.783, 0.905, 0.826 and 0.795 respectively. FIG. 16B shows Precision/Recall curve of the 9 machine learning classifiers using gene expression data of the 142 gene features, and clinical characteristics data of the 3 clinical characteristics to distinguish malignant lung nodules versus benign lung nodules. AUC of the Precision/Recall curve for the 9 machine learning classifiers LOG, RF, SVM, DTREE, ADB, NB, LDA, kNN, and GBM, are 0.854, 0.780, 0.756, 0.632, 0.619, 0.663, 0.754, 0.764 and 0.687 respectively. FIG. 16C shows the tabulated results of the 9 machine learning classifiers corresponding to FIG. 16A. FIG. 16D presents the tabulated results of the 9 machine learning classifiers corresponding to FIG. 16A, with oversampling correction applied (e.g. 80 sample with benign lung nodule, and 80 samples with malignant lung nodule). As can be seen from FIGS. 16C and D relatively high predictive value can achieved using the set 142 gene features (Table 5), and a set of 3 clinical characteristics NCNSZE, NCNUPYN, and AGE as features. The top two contributors or predictors or features were nodule size and BCAT1 gene. Table 7 shows the top 34 predictors obtained from the machine learning classifier using the combined dataset of Example 5. Table 7 contains 31 lung-disease associated genes and 3 clinical characteristics (e.g. NCNSZE, NCNUPYN, and AGE).

TABLE 7
Top 34 predictors from Example 5
Predictors
NCNSZE
BCAT1
CRCP
COA4
OVCA2
POM121
HLA-DPA1
VPS37C
AGE
MGST2
RNF220
HDAC3
NFE2L1
WDR20
CNPY4
HOXB2
C6orf120
TMEM8A
ASAP1-IT2
C15orf54
CD101
FNBP1
TECR
PROK2
SLC35B3
TDRD9
CLHC1
LPL
NCNUPYN
IFITM3
OGFOD3
EIF2B3
TMEM65
MKRN3

Next, the top 34 predictors were examined for their effectiveness in predicting lung nodules. A biomarker data set for the top 34 predictors were obtained from the 152 subjects. As described above, among those, 80 subjects had a diagnosis of a benign lung nodule, and 72 subjects had a diagnosis of a malignant lung nodule. The top 34 predictors contains 31 genes and NCNSZE (nodule size), NCNUPYN (nodule in the upper lobe), and AGE, as predictors.

FIG. 17A shows ROC plots showing performance of the 9 machine learning classifiers using measurement data (e.g. gene expression data or clinical characteristics data as appropriate) of the 34 predictors to distinguish malignant lung nodules versus benign lung nodules. 10-fold cross validation using an 80% training and 20% validation split of the biomarkers dataset was used. AUC of the ROC plots for the 9 machine learning classifiers LOG, RF, SVM, DTREE, ADB, NB, LDA, kNN, and GBM, are 0.992, 0.867, 0.950, 0.675, 0.800, 0.854, 0.963, 0.835 and 0.842 respectively. FIG. 17B shows Precision/Recall curve of the 9 machine learning classifiers using measurement data of the 34 predictors to distinguish malignant lung nodules versus benign lung nodules. AUC of the Precision/Recall curve for the 9 machine learning classifiers LOG, RF, SVM, DTREE, ADB, NB, LDA, kNN, and GBM, are 0.988, 0.807, 0.931, 0.687, 0.747, 0.815, 0.943, 0.814 and 0.811 respectively. FIG. 17C presents the tabulated results of the machine learning classifiers LOG and RF corresponding to FIG. 17A. FIG. 17D presents the tabulated results of the 9 machine learning classifiers corresponding to FIG. 17A, with oversampling correction applied (e.g. 80 sample with benign lung nodule, and 80 samples with malignant lung nodule). FIG. 17E shows feature importance of the 34 features for all the 9 classifiers. As can be seen from FIGS. 17C and D relatively high predictive value can achieved using the 34 predictors containing the set of genes and clinical characteristics of Table 7.

### Example 6: Machine Learning Classification Using Gene Expression Data and Clinical Characteristics Data

A combination of a set of 175 gene features (Table 2), and a set of 4 clinical characteristics features were examined for their effectiveness in predicting lung nodules. The 175 gene features were selected based on results of Examples 1, 2 and 3. The 4 clinical characteristics features, NCNSZE (nodule size), NCNUPYN (nodule in the upper lobe), AGE, and NCNMYN (Nodule Spiculated), were selected based on the results of Example 4. Gene expression measurements were from whole blood samples of the subjects. A combined biomarker dataset containing measurement data of the 179 features (e.g. 175 gene features and 4 clinical characteristics features) from the 152 subjects was analyzed. As described above, among those, 80 subjects had a diagnosis of a benign lung nodule, and 72 subjects had a diagnosis of a malignant lung nodule.

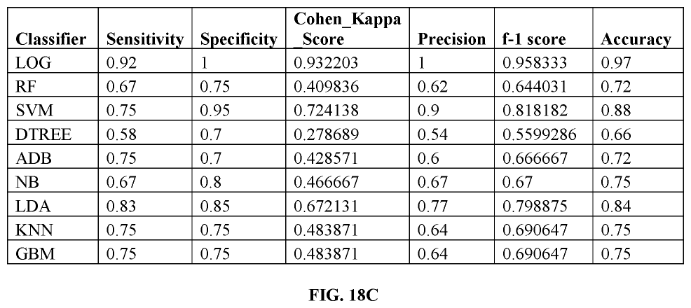

FIG. 18A shows ROC plots showing performance of the 9 machine learning classifiers using gene expression data of the 175 gene features, and a clinical characteristics data of the 4 clinical characteristics to distinguish malignant lung nodules versus benign lung nodules. 10-fold cross validation using an 80% training and 20% validation split of the combined biomarkers dataset was used. AUC of the ROC plots for the 9 machine learning classifiers LOG, RF, SVM, DTREE, ADB, NB, LDA, kNN, and GBM, are 0.674, 0.698, 0.669, 0.702, 0.723, 0.657, 0.630, 0.560 and 0.784 respectively. FIG. 18B shows Precision/Recall curve of the 9 machine learning classifiers using gene expression data of the 175 gene features, and a clinical characteristics data of the 4 clinical characteristics to distinguish malignant lung nodules versus benign lung nodules. AUC of the Precision/Recall curve for the 9 machine learning classifiers LOG, RF, SVM, DTREE, ADB, NB, LDA, kNN, and GBM, are 0.635, 0.724, 0.664, 0.727, 0.663, 0.630, 0.544, 0.550 and 0.729 respectively. FIG. 18C shows the tabulated results of the 9 machine learning classifiers corresponding to FIG. 18A. Table 8 shows the top 22 predictors obtained from the machine learning classifier using the combined dataset of Example 6.

TABLE 8
Top 22 predictors from Example 6
Predictors
NCNSZE
BCAT1
USP32P2
CD177
QPCT
SCAF4
SNRPD3
BCL9L
THBS1
SLC22A18AS
ARCN1
DHX16
SATB1
ST6GAL1
CXCL1
TDRD9
ZNF831
MTCH1
FAM86HP
DHX8
RNF114
DCTN4

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for assessing a lung nodule of a patient, the method comprising:
a) obtaining a data set comprising i) gene expression measurements of a biological sample from the patient, of at least two lung disease-associated genes selected from the group of genes listed in Table 4, Table 7 or both, and ii) clinical characteristics data of one or more clinical characteristics of the patient, selected from the group of clinical characteristics listed in Table 6, wherein the biological sample is a blood sample, isolated peripheral blood mononuclear cells (PBMCs), or any derivative thereof;
b) providing the data set as an input to a machine-learning model trained to generate an inference of whether the data set is indicative of a malignant lung nodule or a benign lung nodule;
c) receiving, as an output of the machine-learning model, the inference indicating whether the data set is indicative of the malignant lung nodule or the benign lung nodule; and
d) electronically outputting a report classifying the lung nodule of the patient as the malignant lung nodule or the benign lung nodule.

2. The method of claim 1, wherein the at least two lung disease-associated genes are selected from the group of genes listed in Table 7.

3. The method of claim 1 or 2, wherein the one or more clinical characteristics comprises size of the nodule, age of the patient, and presence of the nodule in the lung upper lobe.

4. The method of any one of claims 1 to 3, wherein the machine-learning model is developed using a linear regression, a logistic regression (LOG), a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, a linear discriminant analysis (LDA), a decision tree learning (DTREE), an adaptive boosting (ADB), or any combination thereof.

5. The method of any one of claims 1 to 4, wherein the patient has lung cancer.

6. The method of any one of claims 1 to 4, wherein the patient does not have lung cancer.

7. The method of any one of claims 1 to 4, wherein the patient is at an elevated risk of having lung cancer.

8. The method of any one of claims 1 to 5 and 7, wherein the patient is asymptomatic for lung cancer.

9. The method of any one of claims 1 to 5, 7 and 8, further comprising administering a treatment based on the patient's nodule being classified as a malignant nodule.

10. The method of claim 9, wherein the treatment is surgery, chemotherapy, targeted therapy, immunotherapy, radiotherapy, or any combination thereof.

11. The method of any one of claims 1 to 10, wherein the inference includes a confidence value between 0 and 1 that the lung nodule is malignant.

12. The method of any one of claims 1 to 11, wherein the at least two lung disease-associated genes comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, or 295, genes selected from the group of genes listed in Table 4.

13. The method of any one of claims 1 to 12, wherein the at least two lung disease-associated genes comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31, genes selected from the group of genes listed in Table 7.

14. The method of any one of claims 1 to 13, comprising classifying the lung nodule of the patient as the malignant lung nodule or the benign lung nodule with an accuracy of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

15. The method of any one of claims 1 to 14, comprising classifying the lung nodule of the patient as the malignant lung nodule or the benign lung nodule with a sensitivity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

16. The method of any one of claims 1 to 15, comprising classifying the lung nodule of the patient as the malignant lung nodule or the benign lung nodule with a specificity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

17. The method of any one of claims 1 to 16, comprising classifying the lung nodule of the patient as the malignant lung nodule or the benign lung nodule with a positive predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

18. The method of any one of claims 1 to 17, comprising classifying the lung nodule of the patient as the malignant lung nodule or the benign lung nodule with a negative predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

19. The method of any one of claims 1 to 18, wherein the trained machine learning model has a receiver operating characteristic (ROC) curve with an Area-Under-Curve (AUC) of at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

20. A system for assessing a lung nodule of a patient, the system comprising:
one or more processors; and
one or more memories storing executable instructions that, as a result of execution by the one or more processors, cause the system to:
obtain, from a data base, a data set comprising i) gene expression measurements of a biological sample of a patient of a plurality of lung disease-associated genes, selected from the group of genes listed in Table 4 or Table 7 or both, and ii) clinical characteristics data of one or more clinical characteristics of the patient, selected from the group of clinical characteristics listed in Table 6, wherein the biological sample is a blood sample, isolated peripheral blood mononuclear cells (PBMCs), or any derivative thereof;
provide the dataset as an input to a machine-learning model trained to generate an inference of whether the data set is indicative of a malignant lung nodule or a benign lung nodule;
receive, as an output of the machine-learning model, the inference indicating whether the composite data set is indicative of the malignant lung nodule or the benign lung nodule; and
generate a report classifying the lung nodule of the patient as the malignant lung nodule or the benign lung nodule.

21. A non-transitory computer-readable medium storing executable instructions for assessing a lung nodule of a patient that, as a result of execution by one or more processors of a computer system, cause the computer system to:
obtain, from a data base, a data set comprising i) gene expression measurements of a biological sample of a patient of a plurality of lung disease-associated genes, selected from the group of genes listed in Table 4, or Table 7 or both and ii) clinical characteristics data of one or more clinical characteristics of the patient, selected from the group of clinical characteristics listed in Table 6, wherein the biological sample is a blood sample, isolated peripheral blood mononuclear cells (PBMCs), or any derivative thereof;
provide the data set as an input to a machine-learning model trained to generate an inference of whether the data set is indicative of a malignant lung nodule or a benign lung nodule;
receive, as an output of the machine-learning model, the inference indicating whether the composite data set is indicative of the malignant lung nodule or the benign lung nodule; and
generate a report classifying the lung nodule of the patient as the malignant lung nodule or the benign lung nodule.

22. A method for determining a gene set capable of classifying a lung nodule benign or malignant without performing biopsy, the method comprising:
a) obtaining a reference data set comprising a plurality of individual reference data sets, wherein a respective individual reference data set of the plurality of individual reference data sets comprises i) gene expression measurements of a plurality of genes of a reference biological sample from a reference subject having a lung nodule, ii) clinical characteristics data of one or more clinical characteristics selected from the group of clinical characteristics listed in Table 6 of the reference subject, and iii) data regarding whether the lung nodule of the reference subject is benign or malignant, wherein the reference biological sample is a blood sample, isolated peripheral blood mononuclear cells (PBMCs), or any derivative thereof;
b) training a machine learning model using the reference data set, wherein the machine learning model is trained to infer whether a lung nodule is benign or malignant based on at least in part on one or more predictors selected from the plurality of genes, and the one or more clinical characteristics;
c) determining feature importance values of the plurality of genes; and
d) determining the gene set based at least in part on the feature importance values.

23. The method of claim 22, wherein the plurality of genes comprises at least 2 genes selected from the group of genes listed in Table 9.

24. A method for developing a trained machine learning model capable of inferring whether a lung nodule of a patient is benign or malignant, the method comprising:
(a) obtaining a first reference data set comprising a plurality of first individual reference data sets, wherein a respective first individual reference data set of the plurality of first individual reference data sets comprises i) gene expression measurements of a plurality of genes of a reference biological sample from a reference subject having a lung nodule, ii) clinical characteristics data of one or more clinical characteristics selected from a group of clinical characteristics listed in Table 6 of the reference subject, and iii) data regarding whether the lung nodule of the reference subject is benign or malignant, wherein the biological sample is a blood sample, isolated peripheral blood mononuclear cells (PBMCs), or any derivative thereof;
(b) training a first machine learning model using the first reference data set, wherein the first machine learning model is trained to infer whether a lung nodule is benign or malignant, based at least in part on one or more predictors selected from the plurality of genes, and the one or more clinical characteristics;
(c) determining feature importance values of the one or more predictors of the first machine learning model;
(d) selecting A predictors of the first machine learning model based at least in part on the feature importance values, wherein A is an integer from 5 to 2000; and
(e) training a second machine learning model based at least in part on a second reference data set comprising a plurality of second individual reference data sets, wherein a respective second individual reference data set of the plurality of second individual reference data sets comprises i) measurement data of the A predictors of the reference subject, and ii) data regarding whether the lung nodule of the reference subject is benign or malignant, to obtain the trained machine learning model, wherein the trained machine learning model is trained to infer whether a lung nodule is benign or malignant, based at least in part on measurement data of the A predictors.

25. The method of claim 24, wherein the plurality of genes comprises at least 2 genes selected from the group of genes listed in Table 9.

26. The method of any one of claims 24 to 25, wherein the A predictors have top 5 to 200 feature importance values.

27. The method of any one of claims 24 to 26, wherein the trained machine learning model has an accuracy of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

28. The method of any one of claims 24 to 27, wherein the trained machine learning model has an sensitivity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

29. The method of any one of claims 24 to 28, wherein the trained machine learning model has an specificity of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

30. The method of any one of claims 24 to 29, wherein the trained machine learning model has a positive predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

31. The method of any one of claims 24 to 30, wherein the trained machine learning model has a negative predictive value of at least about 80%, at least about 85%, at least about 90%, at least about 91%, at least about 92%, at least about 93%, at least about 94%, at least about 95%, at least about 96%, at least about 97%, at least about 98%, at least about 99%, or more than about 99%.

32. The method of any one of claims 24 to 31, wherein the trained machine learning model has a receiver operating characteristic (ROC) curve with an Area-Under-Curve (AUC) of at least about 0.80, at least about 0.85, at least about 0.90, at least about 0.91, at least about 0.92, at least about 0.93, at least about 0.94, at least about 0.95, at least about 0.96, at least about 0.97, at least about 0.98, at least about 0.99, or more than about 0.99.

33. The method of any one of claims 24 to 32, wherein the first machine learning model and second machine learning model is independently trained using a linear regression, a logistic regression (LOG), a Ridge regression, a Lasso regression, an elastic net (EN) regression, a support vector machine (SVM), a gradient boosted machine (GBM), a k nearest neighbors (kNN), a generalized linear model (GLM), a naïve Bayes (NB) classifier, a neural network, a Random Forest (RF), a deep learning algorithm, a linear discriminant analysis (LDA), a decision tree learning (DTREE), an adaptive boosting (ADB), or any combination thereof.

34. A method for assessing a lung nodule of a patient, the method comprising:
(a) obtaining a data set comprising measurement data of the patient of one or more of the A predictors of any one of claims 24 to 26;
(b) providing the data set as an input to a trained machine-learning model trained according to the methods of any one of claims 24 to 33 to generate an inference of whether the data set is indicative a malignant lung nodule or a benign lung nodule;
(c) receiving, as an output of the machine-learning model, the inference indicating whether the data set is indicative of the malignant lung nodule or the benign lung nodule; and
(d) electronically outputting a report classifying the lung nodule of the patient as the malignant lung nodule or the benign lung nodule.

35. The method of claim 34, wherein the biological sample is a blood sample, isolated peripheral blood mononuclear cells (PBMCs), or any derivative thereof.

36. The method of any one of claims 34 to 35, wherein the patient has lung cancer.

37. The method of any one of claims 34 to 35, wherein the patient does not have lung cancer.

38. The method of any one of claims 34 to 35, wherein the patient is at elevated risk of having lung cancer.

39. The method of any one of claims 34 to 36 and 38, wherein the patient is asymptomatic for lung cancer.

40. The method of any one of claims 34 to 36, 38 and 39, further comprising administering a treatment based on the patient's lung nodule being classified as a malignant nodule.

41. The method of claim 40, wherein the treatment is surgery, chemotherapy, targeted therapy, immunotherapy, radiotherapy, or any combination thereof.

42. A method for treating lung cancer in a patient having a lung nodule, the method comprising:
(a) obtaining a data set comprising i) gene expression measurements of a biological sample from the patient, of at least two lung disease-associated genes selected from the group of genes listed in Table 4, or Table 7 or both, and ii) clinical characteristics data of one or more clinical characteristics selected from the group of clinical characteristics listed in Table 6 of the patient, wherein the biological sample is a blood sample, isolated peripheral blood mononuclear cells (PBMCs), or any derivative thereof;
(b) providing the data set as an input to a trained machine-learning model trained to generate an inference of whether the data set is indicative of a malignant lung nodule or a benign lung nodule;
(c) receiving, as an output of the machine-learning model, the inference indicating whether the data set is indicative of the malignant lung nodule or the benign lung nodule; and
(d) administering a treatment based on the patient's lung nodule being classified as the malignant lung nodule.
